SEQUENCE LISTING (43 sequences)

SEQ ID NO: 1 - protein, 17 aa, Myceliophthora thermophila
MYAKFATLAALVAGAAA

SEQ ID NO: 2 - protein, 4 aa, synthetic construct
  REGION 1..4: note = Exemplary purification tag
EPEA

SEQ ID NO: 3 - protein, 540 aa, Newcastle disease virus
MGSKPSTRIPAPLMLITRIMLTLSCIRLTSSLDGRPLAAAGIVVTGDKAVNVYTSSQTGSIIVKLLPNMPRDKEACARAPLEAYNRTLTTLLTPLGDSIRKIQGSVSTSGGRRQKRFIGAVIGSVALGVATAAQITAAAALIQAKQNAANILRLKESIAATNEAVHEVTDGLSQLSVAVGKMQQFVNDQFNNTARELDCIKITQQVGVELNLYLTELTTVFGPQITSPALTQLTIQALYNLAGGNMDYLLTKLGIGNNQLSSLIGSGLITGYPILYDSHTQLLGIQVNLPSVGNLNNMRATYLETLSVSTTKGYASALVPKVVTQVGSVIEELDTSYCIESDLDLYCTRIVTFPMSPGIYSCLSGNTSACMYSKTEGALTTPYMALRGSVIANCKITTCRCTDPPGIISQNYGEAVSLIDRHSCNVLSLDGITLRLSGEFDATYQKNISILDSQVIVTGNLDISTELGNVNNSISNALDRLAESNSKLEKVNVRLTSTSALITYIVLTVISLIFGALSLALACYLMYKQKAQQKTLLWLG

SEQ ID NO: 4 - protein, 571 aa, Newcastle disease virus
MDRAVNRVVLENEEREAKNTWRLVFRIAVLLLMAMILAISAAALAYSMEASTPHDLAGISTVISKTEDKVTSLLSSGQDVIDRIYKQVALESPLALLNTESVIMNAITSLSYQINGAENSSGCGAPVHDPDYIGGIGKELIVDDISDVTSFYPSAYQEHLNFIPAPTTGSGCTRIPSFDMSTTHYCYTHNVILSGCRDHSHSHQYLALGVLRTSATGRVFFSTLRSINLDDTQNRKSCSVSATPLGCDMLCSKVTGTEEEDYKSVAPTSMVHGRLGFDGQYHEKDLDTTVLFKDWVANYPGVGGGSFIDGRVWFPVYGGLKPNSPSDTAQEGKYVIYKRHNNTCPDKQDYQIRMAKSSYKPARFGGKRVQQAILSIKVSTSLGRDPVLTIPPNTITLMGAEGRILTVGTSHFLYQRGSSYFSPALLYPMTVNNKTATLHSPYMFNAFTRPGSVPCQASARCPNSCITGVYTDPYPLIFYRNHTLRGVFGTMLDDEQARLNPVSAVFDNISRSRVTRVSSSSTKAAYTTSTCFKVVKTNKAYCLSIAEISNTLFGEFRIVPLLVEILKDDRV

SEQ ID NO: 5 - DNA, 1632 bp, synthetic construct
  misc_feature 1..1632: note = Exemplary synthetic F protein
  source 1..1632: mol_type = other DNA
atgtacgccaagttcgcgaccctcgccgcccttgtggctggcgccgctgctctggacggccgccccctggctgccgccggcatcgtcgtcaccggcgacaaggccgtcaacgtctacaccagctcgcagaccggctcgatcatcgtcaagctcctgcccaacatgccgcgcgacaaggaagcctgcgcccgcgccccgctcgaggcctacaaccgcaccctcaccaccctgctgaccccgctcggcgactccatccgcaagatccagggctccgtctccaccagcggcggccggcgccagaagcgcttcatcggcgccgtcatcggctcggtcgccctgggcgtcgccaccgccgcccagatcaccgctgccgccgccctcatccaggccaagcagaacgccgccaacatcctccgcctgaaggagtcgatcgccgccaccaacgaggccgtccacgaggtcaccgacggcctcagccagctgtccgtcgccgtcggcaagatgcagcagttcgtcaacgaccagttcaacaacaccgcccgcgagctggactgcatcaagatcacccagcaggtcggcgtcgagctcaacctgtacctcaccgagctcaccaccgtcttcggcccccagatcacctccccggccctcacccagctgaccatccaggccctctacaacctggccggcggcaacatggactacctcctgaccaagctcggcatcggcaacaaccagctctccagcctgatcggctcgggcctgatcaccggctaccccatcctctacgactcccacacccagctcctgggcatccaggtcaacctcccgagcgtcggcaacctgaacaacatgcgcgccacctacctcgagaccctgtcggtctcgaccaccaagggctacgcctcggccctggtccccaaggtcgtcacccaggtcggctcggtcatcgaggagctcgacaccagctactgcatcgagtcggacctggacctctactgcacccgcatcgtcaccttccccatgagccccggcatctactcctgcctcagcggcaacacctccgcctgcatgtactccaagaccgagggcgccctgaccaccccctacatggccctccgcggctccgtcatcgccaactgcaagatcaccacctgccgctgcaccgacccgcccggcatcatctcccagaactacggcgaggccgtcagcctgatcgaccgccactcgtgcaacgtcctgtcgctggacggcatcaccctgcgcctctccggcgagttcgacgccacctaccagaagaacatctcgatcctcgactcccaggtcatcgtcaccggcaacctcgacatctccaccgagctgggcaacgtcaacaactcgatctccaacgccctggaccgcctggccgagtccaacagcaagctcgagaaggtcaacgtccgcctcacctcgacctccgccctgatcacctacatcgtcctcaccgtcatctcgctcatcttcggcgccctgtccctggccctggcctgctacctcatgtacaagcagaaggcccagcagaagaccctcctgtggctcggcaacaacaccctggaccagatgcgcgccaccacccgcgccgagccggaggcctaa

SEQ ID NO: 6 - protein, 543 aa, synthetic construct
  REGION 1..543: note = Exemplary synthetic F protein
MYAKFATLAALVAGAAALDGRPLAAAGIVVTGDKAVNVYTSSQTGSIIVKLLPNMPRDKEACARAPLEAYNRTLTTLLTPLGDSIRKIQGSVSTSGGRRQKRFIGAVIGSVALGVATAAQITAAAALIQAKQNAANILRLKESIAATNEAVHEVTDGLSQLSVAVGKMQQFVNDQFNNTARELDCIKITQQVGVELNLYLTELTTVFGPQITSPALTQLTIQALYNLAGGNMDYLLTKLGIGNNQLSSLIGSGLITGYPILYDSHTQLLGIQVNLPSVGNLNNMRATYLETLSVSTTKGYASALVPKVVTQVGSVIEELDTSYCIESDLDLYCTRIVTFPMSPGIYSCLSGNTSACMYSKTEGALTTPYMALRGSVIANCKITTCRCTDPPGIISQNYGEAVSLIDRHSCNVLSLDGITLRLSGEFDATYQKNISILDSQVIVTGNLDISTELGNVNNSISNALDRLAESNSKLEKVNVRLTSTSALITYIVLTVISLIFGALSLALACYLMYKQKAQQKTLLWLGNNTLDQMRATTRAEPEA

SEQ ID NO: 7 - protein, 526 aa, synthetic construct
  REGION 1..526: note = Exemplary synthetic F protein
LDGRPLAAAGIVVTGDKAVNVYTSSQTGSIIVKLLPNMPRDKEACARAPLEAYNRTLTTLLTPLGDSIRKIQGSVSTSGGRRQKRFIGAVIGSVALGVATAAQITAAAALIQAKQNAANILRLKESIAATNEAVHEVTDGLSQLSVAVGKMQQFVNDQFNNTARELDCIKITQQVGVELNLYLTELTTVFGPQITSPALTQLTIQALYNLAGGNMDYLLTKLGIGNNQLSSLIGSGLITGYPILYDSHTQLLGIQVNLPSVGNLNNMRATYLETLSVSTTKGYASALVPKVVTQVGSVIEELDTSYCIESDLDLYCTRIVTFPMSPGIYSCLSGNTSACMYSKTEGALTTPYMALRGSVIANCKITTCRCTDPPGIISQNYGEAVSLIDRHSCNVLSLDGITLRLSGEFDATYQKNISILDSQVIVTGNLDISTELGNVNNSISNALDRLAESNSKLEKVNVRLTSTSALITYIVLTVISLIFGALSLALACYLMYKQKAQQKTLLWLGNNTLDQMRATTRAEPEA

SEQ ID NO: 8 - DNA, 1470 bp, synthetic construct
  misc_feature 1..1470: note = Exemplary synthetic F protein
  source 1..1470: mol_type = other DNA
atgtacgccaagttcgcgaccctcgccgcccttgtggctggcgccgctgctctggacggccgccccctggctgccgccggcatcgtcgtcaccggcgacaaggccgtcaacgtctacaccagctcgcagaccggctcgatcatcgtcaagctcctgcccaacatgccgcgcgacaaggaagcctgcgcccgcgccccgctcgaggcctacaaccgcaccctcaccaccctgctgaccccgctcggcgactccatccgcaagatccagggctccgtctccaccagcggcggccggcgccagaagcgcttcatcggcgccgtcatcggctcggtcgccctgggcgtcgccaccgccgcccagatcaccgctgccgccgccctcatccaggccaagcagaacgccgccaacatcctccgcctgaaggagtcgatcgccgccaccaacgaggccgtccacgaggtcaccgacggcctcagccagctgtccgtcgccgtcggcaagatgcagcagttcgtcaacgaccagttcaacaacaccgcccgcgagctggactgcatcaagatcacccagcaggtcggcgtcgagctcaacctgtacctcaccgagctcaccaccgtcttcggcccccagatcacctccccggccctcacccagctgaccatccaggccctctacaacctggccggcggcaacatggactacctcctgaccaagctcggcatcggcaacaaccagctctccagcctgatcggctcgggcctgatcaccggctaccccatcctctacgactcccacacccagctcctgggcatccaggtcaacctcccgagcgtcggcaacctgaacaacatgcgcgccacctacctcgagaccctgtcggtctcgaccaccaagggctacgcctcggccctggtccccaaggtcgtcacccaggtcggctcggtcatcgaggagctcgacaccagctactgcatcgagtcggacctggacctctactgcacccgcatcgtcaccttccccatgagccccggcatctactcctgcctcagcggcaacacctccgcctgcatgtactccaagaccgagggcgccctgaccaccccctacatggccctccgcggctccgtcatcgccaactgcaagatcaccacctgccgctgcaccgacccgcccggcatcatctcccagaactacggcgaggccgtcagcctgatcgaccgccactcgtgcaacgtcctgtcgctggacggcatcaccctgcgcctctccggcgagttcgacgccacctaccagaagaacatctcgatcctcgactcccaggtcatcgtcaccggcaacctcgacatctccaccgagctgggcaacgtcaacaactcgatctccaacgccctggaccgcctggccgagtccaacagcaagctcgagaaggtcaacgtccgcctcacctcgacctccgagccggaggcctaa

SEQ ID NO: 9 - protein, 489 aa, synthetic construct
  REGION 1..489: note = pMYT1359_amino_acid
MYAKFATLAALVAGAAALDGRPLAAAGIVVTGDKAVNVYTSSQTGSIIVKLLPNMPRDKEACARAPLEAYNRTLTTLLTPLGDSIRKIQGSVSTSGGRRQKRFIGAVIGSVALGVATAAQITAAAALIQAKQNAANILRLKESIAATNEAVHEVTDGLSQLSVAVGKMQQFVNDQFNNTARELDCIKITQQVGVELNLYLTELTTVFGPQITSPALTQLTIQALYNLAGGNMDYLLTKLGIGNNQLSSLIGSGLITGYPILYDSHTQLLGIQVNLPSVGNLNNMRATYLETLSVSTTKGYASALVPKVVTQVGSVIEELDTSYCIESDLDLYCTRIVTFPMSPGIYSCLSGNTSACMYSKTEGALTTPYMALRGSVIANCKITTCRCTDPPGIISQNYGEAVSLIDRHSCNVLSLDGITLRLSGEFDATYQKNISILDSQVIVTGNLDISTELGNVNNSISNALDRLAESNSKLEKVNVRLTSTSEPEA

SEQ ID NO: 10 - protein, 472 aa, synthetic construct
  REGION 1..472: note = pMYT1359_mature_protein
LDGRPLAAAGIVVTGDKAVNVYTSSQTGSIIVKLLPNMPRDKEACARAPLEAYNRTLTTLLTPLGDSIRKIQGSVSTSGGRRQKRFIGAVIGSVALGVATAAQITAAAALIQAKQNAANILRLKESIAATNEAVHEVTDGLSQLSVAVGKMQQFVNDQFNNTARELDCIKITQQVGVELNLYLTELTTVFGPQITSPALTQLTIQALYNLAGGNMDYLLTKLGIGNNQLSSLIGSGLITGYPILYDSHTQLLGIQVNLPSVGNLNNMRATYLETLSVSTTKGYASALVPKVVTQVGSVIEELDTSYCIESDLDLYCTRIVTFPMSPGIYSCLSGNTSACMYSKTEGALTTPYMALRGSVIANCKITTCRCTDPPGIISQNYGEAVSLIDRHSCNVLSLDGITLRLSGEFDATYQKNISILDSQVIVTGNLDISTELGNVNNSISNALDRLAESNSKLEKVNVRLTSTSEPEA

SEQ ID NO: 11 - DNA, 1128 bp, synthetic construct
  misc_feature 1..1128: note = pMYT1360_CDS_DNA
  source 1..1128: mol_type = other DNA
atgtacgccaagttcgcgaccctcgccgcccttgtggctggcgccgctgctcagaacgccgccaacatcctccgcctgaaggagtcgatcgccgccaccaacgaggccgtccacgaggtcaccgacggcctcagccagctgtccgtcgccgtcggcaagatgcagcagttcgtcaacgaccagttcaacaacaccgcccgcgagctggactgcatcaagatcacccagcaggtcggcgtcgagctcaacctgtacctcaccgagctcaccaccgtcttcggcccccagatcacctccccggccctcacccagctgaccatccaggccctctacaacctggccggcggcaacatggactacctcctgaccaagctcggcatcggcaacaaccagctctccagcctgatcggctcgggcctgatcaccggctaccccatcctctacgactcccacacccagctcctgggcatccaggtcaacctcccgagcgtcggcaacctgaacaacatgcgcgccacctacctcgagaccctgtcggtctcgaccaccaagggctacgcctcggccctggtccccaaggtcgtcacccaggtcggctcggtcatcgaggagctcgacaccagctactgcatcgagtcggacctggacctctactgcacccgcatcgtcaccttccccatgagccccggcatctactcctgcctcagcggcaacacctccgcctgcatgtactccaagaccgagggcgccctgaccaccccctacatggccctccgcggctccgtcatcgccaactgcaagatcaccacctgccgctgcaccgacccgcccggcatcatctcccagaactacggcgaggccgtcagcctgatcgaccgccactcgtgcaacgtcctgtcgctggacggcatcaccctgcgcctctccggcgagttcgacgccacctaccagaagaacatctcgatcctcgactcccaggtcatcgtcaccggcaacctcgacatctccaccgagctgggcaacgtcaacaactcgatctccaacgccctggaccgcctggccgagtccaacagcaagctcgagaaggtcaacgtccgcctcacctcgacctccgagccggaggcctaa

SEQ ID NO: 12 - protein, 375 aa, synthetic construct
  REGION 1..375: note = pMYT1360_amino_acid
MYAKFATLAALVAGAAAQNAANILRLKESIAATNEAVHEVTDGLSQLSVAVGKMQQFVNDQFNNTARELDCIKITQQVGVELNLYLTELTTVFGPQITSPALTQLTIQALYNLAGGNMDYLLTKLGIGNNQLSSLIGSGLITGYPILYDSHTQLLGIQVNLPSVGNLNNMRATYLETLSVSTTKGYASALVPKVVTQVGSVIEELDTSYCIESDLDLYCTRIVTFPMSPGIYSCLSGNTSACMYSKTEGALTTPYMALRGSVIANCKITTCRCTDPPGIISQNYGEAVSLIDRHSCNVLSLDGITLRLSGEFDATYQKNISILDSQVIVTGNLDISTELGNVNNSISNALDRLAESNSKLEKVNVRLTSTSEPEA

SEQ ID NO: 13 - protein, 358 aa, synthetic construct
  REGION 1..358: note = pMYT1360_mature_protein
QNAANILRLKESIAATNEAVHEVTDGLSQLSVAVGKMQQFVNDQFNNTARELDCIKITQQVGVELNLYLTELTTVFGPQITSPALTQLTIQALYNLAGGNMDYLLTKLGIGNNQLSSLIGSGLITGYPILYDSHTQLLGIQVNLPSVGNLNNMRATYLETLSVSTTKGYASALVPKVVTQVGSVIEELDTSYCIESDLDLYCTRIVTFPMSPGIYSCLSGNTSACMYSKTEGALTTPYMALRGSVIANCKITTCRCTDPPGIISQNYGEAVSLIDRHSCNVLSLDGITLRLSGEFDATYQKNISILDSQVIVTGNLDISTELGNVNNSISNALDRLAESNSKLEKVNVRLTSTSEPEA

SEQ ID NO: 14 - DNA, 1470 bp, synthetic construct
  misc_feature 1..1470: note = pMYT1361_CDS_DNA
  source 1..1470: mol_type = other DNA
atgtacgccaagttcgcgaccctcgccgcccttgtggctggcgccgctgctctggacggccgccccctggctgccgccggcatcgtcgtcaccggcgacaaggccgtcaacgtctacaccagctcgcagaccggctcgatcatcgtcaagctcctgcccaacatgccgcgcgacaaggaagcctgcgcccgcgccccgctcgaggcctacaaccgcaccctcaccaccctgctgaccccgctcggcgactccatccgcaagatccagggctccgtctccaccagcggcggccggcgccagggccgcttcatcggcgccgtcatcggctcggtcgccctgggcgtcgccaccgccgcccagatcaccgctgccgccgccctcatccaggccaagcagaacgccgccaacatcctccgcctgaaggagtcgatcgccgccaccaacgaggccgtccacgaggtcaccgacggcctcagccagctgtccgtcgccgtcggcaagatgcagcagttcgtcaacgaccagttcaacaacaccgcccgcgagctggactgcatcaagatcacccagcaggtcggcgtcgagctcaacctgtacctcaccgagctcaccaccgtcttcggcccccagatcacctccccggccctcacccagctgaccatccaggccctctacaacctggccggcggcaacatggactacctcctgaccaagctcggcatcggcaacaaccagctctccagcctgatcggctcgggcctgatcaccggctaccccatcctctacgactcccacacccagctcctgggcatccaggtcaacctcccgagcgtcggcaacctgaacaacatgcgcgccacctacctcgagaccctgtcggtctcgaccaccaagggctacgcctcggccctggtccccaaggtcgtcacccaggtcggctcggtcatcgaggagctcgacaccagctactgcatcgagtcggacctggacctctactgcacccgcatcgtcaccttccccatgagccccggcatctactcctgcctcagcggcaacacctccgcctgcatgtactccaagaccgagggcgccctgaccaccccctacatggccctccgcggctccgtcatcgccaactgcaagatcaccacctgccgctgcaccgacccgcccggcatcatctcccagaactacggcgaggccgtcagcctgatcgaccgccactcgtgcaacgtcctgtcgctggacggcatcaccctgcgcctctccggcgagttcgacgccacctaccagaagaacatctcgatcctcgactcccaggtcatcgtcaccggcaacctcgacatctccaccgagctgggcaacgtcaacaactcgatctccaacgccctggaccgcctggccgagtccaacagcaagctcgagaaggtcaacgtccgcctcacctcgacctccgagccggaggcctaa

SEQ ID NO: 15 - protein, 489 aa, synthetic construct
  REGION 1..489: note = pMYT1361_amino_acid
MYAKFATLAALVAGAAALDGRPLAAAGIVVTGDKAVNVYTSSQTGSIIVKLLPNMPRDKEACARAPLEAYNRTLTTLLTPLGDSIRKIQGSVSTSGGRRQGRFIGAVIGSVALGVATAAQITAAAALIQAKQNAANILRLKESIAATNEAVHEVTDGLSQLSVAVGKMQQFVNDQFNNTARELDCIKITQQVGVELNLYLTELTTVFGPQITSPALTQLTIQALYNLAGGNMDYLLTKLGIGNNQLSSLIGSGLITGYPILYDSHTQLLGIQVNLPSVGNLNNMRATYLETLSVSTTKGYASALVPKVVTQVGSVIEELDTSYCIESDLDLYCTRIVTFPMSPGIYSCLSGNTSACMYSKTEGALTTPYMALRGSVIANCKITTCRCTDPPGIISQNYGEAVSLIDRHSCNVLSLDGITLRLSGEFDATYQKNISILDSQVIVTGNLDISTELGNVNNSISNALDRLAESNSKLEKVNVRLTSTSEPEA

SEQ ID NO: 16 - protein, 472 aa, synthetic construct
  REGION 1..472: note = pMYT1361_mature_protein
LDGRPLAAAGIVVTGDKAVNVYTSSQTGSIIVKLLPNMPRDKEACARAPLEAYNRTLTTLLTPLGDSIRKIQGSVSTSGGRRQGRFIGAVIGSVALGVATAAQITAAAALIQAKQNAANILRLKESIAATNEAVHEVTDGLSQLSVAVGKMQQFVNDQFNNTARELDCIKITQQVGVELNLYLTELTTVFGPQITSPALTQLTIQALYNLAGGNMDYLLTKLGIGNNQLSSLIGSGLITGYPILYDSHTQLLGIQVNLPSVGNLNNMRATYLETLSVSTTKGYASALVPKVVTQVGSVIEELDTSYCIESDLDLYCTRIVTFPMSPGIYSCLSGNTSACMYSKTEGALTTPYMALRGSVIANCKITTCRCTDPPGIISQNYGEAVSLIDRHSCNVLSLDGITLRLSGEFDATYQKNISILDSQVIVTGNLDISTELGNVNNSISNALDRLAESNSKLEKVNVRLTSTSEPEA

SEQ ID NO: 17 - DNA, 1728 bp, synthetic construct
  misc_feature 1..1728: note = pMYT1362_CDS_DNA
  source 1..1728: mol_type = other DNA
atggaccgcgccgtcaaccgcgtcgtcctcgagaacgaggagcgcgaggccaagaacacctggcgcctggtcttccgcatcgccgtcctcctgctcatggccatgatcctggccatctcggctgccgccctggcctactccatggaggcctccaccccgcacgacctggccggcatcagcaccgtcatctcgaagaccgaggacaaggtcacctcgctgctcagctcgggccaggacgtcatcgaccgcatctacaagcaggtcgccctggagtcgccgctcgccctgctcaacaccgagtccgtcatcatgaacgccatcaccagcctgtcgtaccagatcaacggcgccgagaactcctcgggctgcggcgcccctgtccacgaccccgactacatcggcggcatcggcaaggagctcatcgtcgacgacatctccgacgtcaccagcttctacccctcggcctaccaggagcacctgaacttcatcccggcccccaccaccggctcgggctgcacccgcatcccgtccttcgacatgagcaccacccactactgctacacccacaacgtcatcctctcgggctgccgcgaccactcgcactcgcaccagtacctggccctgggcgtcctgcgcacctcggccaccggccgcgtcttcttctcgaccctgcgctccatcaacctcgacgacacccagaaccgcaagtcctgctcggtctcggccaccccgctcggctgcgacatgctctgctccaaggtcaccggcaccgaggaagaggactacaagtcggtcgcccccacctccatggtccacggccgcctgggcttcgacggccagtaccacgagaaggacctggacaccaccgtcctcttcaaggactgggtcgccaactacccgggcgtcggcggcggcagcttcatcgacggccgcgtctggttccccgtctacggtggcctgaagccgaactccccctcggacaccgcccaggaaggcaagtacgtcatctacaagcgccacaacaacacctgccccgacaagcaggactaccagatccgcatggccaagtcgtcctacaagcccgcccgcttcggtggcaagcgcgtccagcaggccatcctctcgatcaaggtctccacctcgctgggccgcgacccggtcctcaccatcccgcccaacaccatcaccctgatgggcgccgagggccgcatcctcaccgtcggcaccagccacttcctgtaccagcgcggcagctcctacttctcgcccgccctgctctacccgatgaccgtcaacaacaagaccgccaccctccacagcccctacatgttcaacgccttcaccaggcccggctccgtcccctgccaggcctcggcccgctgccccaacagctgcatcaccggcgtctacaccgacccctacccgctcatcttctaccgcaaccacaccctccgcggcgtcttcggcaccatgctcgacgacgagcaggcccgcctcaaccccgtctccgccgtcttcgacaacatctcccgcagccgcgtcacccgcgtctccagctcctccaccaaggccgcctacaccacctccacctgcttcaaggtcgtcaagaccaacaaggcctactgcctctccatcgccgagatcagcaacaccctgttcggcgagttccgcatcgtccccctgctcgtcgagatcctcaaggacgaccgcgtcgagccggaggcctaa

SEQ ID NO: 18 - protein, 575 aa, synthetic construct
  REGION 1..575: note = pMYT1362_amino_acid
MDRAVNRVVLENEEREAKNTWRLVFRIAVLLLMAMILAISAAALAYSMEASTPHDLAGISTVISKTEDKVTSLLSSGQDVIDRIYKQVALESPLALLNTESVIMNAITSLSYQINGAENSSGCGAPVHDPDYIGGIGKELIVDDISDVTSFYPSAYQEHLNFIPAPTTGSGCTRIPSFDMSTTHYCYTHNVILSGCRDHSHSHQYLALGVLRTSATGRVFFSTLRSINLDDTQNRKSCSVSATPLGCDMLCSKVTGTEEEDYKSVAPTSMVHGRLGFDGQYHEKDLDTTVLFKDWVANYPGVGGGSFIDGRVWFPVYGGLKPNSPSDTAQEGKYVIYKRHNNTCPDKQDYQIRMAKSSYKPARFGGKRVQQAILSIKVSTSLGRDPVLTIPPNTITLMGAEGRILTVGTSHFLYQRGSSYFSPALLYPMTVNNKTATLHSPYMFNAFTRPGSVPCQASARCPNSCITGVYTDPYPLIFYRNHTLRGVFGTMLDDEQARLNPVSAVFDNISRSRVTRVSSSSTKAAYTTSTCFKVVKTNKAYCLSIAEISNTLFGEFRIVPLLVEILKDDRVEPEA

SEQ ID NO: 19 - protein, 575 aa, synthetic construct; identical to SEQ ID NO: 18
  REGION 1..575: note = pMYT1362_mature_protein

SEQ ID NO: 20 - DNA, 1779 bp, synthetic construct
  misc_feature 1..1779: note = pMYT1362_CDS_DNA
  source 1..1779: mol_type = other DNA
atgtacgccaagttcgcgaccctcgccgcccttgtggctggcgccgctgctatggaccgcgccgtcaaccgcgtcgtcctcgagaacgaggagcgcgaggccaagaacacctggcgcctggtcttccgcatcgccgtcctcctgctcatggccatgatcctggccatctcggctgccgccctggcctactccatggaggcctccaccccgcacgacctggccggcatcagcaccgtcatctcgaagaccgaggacaaggtcacctcgctgctcagctcgggccaggacgtcatcgaccgcatctacaagcaggtcgccctggagtcgccgctcgccctgctcaacaccgagtccgtcatcatgaacgccatcaccagcctgtcgtaccagatcaacggcgccgagaactcctcgggctgcggcgcccctgtccacgaccccgactacatcggcggcatcggcaaggagctcatcgtcgacgacatctccgacgtcaccagcttctacccctcggcctaccaggagcacctgaacttcatcccggcccccaccaccggctcgggctgcacccgcatcccgtccttcgacatgagcaccacccactactgctacacccacaacgtcatcctctcgggctgccgcgaccactcgcactcgcaccagtacctggccctgggcgtcctgcgcacctcggccaccggccgcgtcttcttctcgaccctgcgctccatcaacctcgacgacacccagaaccgcaagtcctgctcggtctcggccaccccgctcggctgcgacatgctctgctccaaggtcaccggcaccgaggaagaggactacaagtcggtcgcccccacctccatggtccacggccgcctgggcttcgacggccagtaccacgagaaggacctggacaccaccgtcctcttcaaggactgggtcgccaactacccgggcgtcggcggcggcagcttcatcgacggccgcgtctggttccccgtctacggtggcctgaagccgaactccccctcggacaccgcccaggaaggcaagtacgtcatctacaagcgccacaacaacacctgccccgacaagcaggactaccagatccgcatggccaagtcgtcctacaagcccgcccgcttcggtggcaagcgcgtccagcaggccatcctctcgatcaaggtctccacctcgctgggccgcgacccggtcctcaccatcccgcccaacaccatcaccctgatgggcgccgagggccgcatcctcaccgtcggcaccagccacttcctgtaccagcgcggcagctcctacttctcgcccgccctgctctacccgatgaccgtcaacaacaagaccgccaccctccacagcccctacatgttcaacgccttcaccaggcccggctccgtcccctgccaggcctcggcccgctgccccaacagctgcatcaccggcgtctacaccgacccctacccgctcatcttctaccgcaaccacaccctccgcggcgtcttcggcaccatgctcgacgacgagcaggcccgcctcaaccccgtctccgccgtcttcgacaacatctcccgcagccgcgtcacccgcgtctccagctcctccaccaaggccgcctacaccacctccacctgcttcaaggtcgtcaagaccaacaaggcctactgcctctccatcgccgagatcagcaacaccctgttcggcgagttccgcatcgtccccctgctcgtcgagatcctcaaggacgaccgcgtcgagccggaggcctaa

SEQ ID NO: 21 - protein, 592 aa, synthetic construct
  REGION 1..592: note = pMYT1363_amino_acid
MYAKFATLAALVAGAAAMDRAVNRVVLENEEREAKNTWRLVFRIAVLLLMAMILAISAAALAYSMEASTPHDLAGISTVISKTEDKVTSLLSSGQDVIDRIYKQVALESPLALLNTESVIMNAITSLSYQINGAENSSGCGAPVHDPDYIGGIGKELIVDDISDVTSFYPSAYQEHLNFIPAPTTGSGCTRIPSFDMSTTHYCYTHNVILSGCRDHSHSHQYLALGVLRTSATGRVFFSTLRSINLDDTQNRKSCSVSATPLGCDMLCSKVTGTEEEDYKSVAPTSMVHGRLGFDGQYHEKDLDTTVLFKDWVANYPGVGGGSFIDGRVWFPVYGGLKPNSPSDTAQEGKYVIYKRHNNTCPDKQDYQIRMAKSSYKPARFGGKRVQQAILSIKVSTSLGRDPVLTIPPNTITLMGAEGRILTVGTSHFLYQRGSSYFSPALLYPMTVNNKTATLHSPYMFNAFTRPGSVPCQASARCPNSCITGVYTDPYPLIFYRNHTLRGVFGTMLDDEQARLNPVSAVFDNISRSRVTRVSSSSTKAAYTTSTCFKVVKTNKAYCLSIAEISNTLFGEFRIVPLLVEILKDDRVEPEA

SEQ ID NO: 22 - protein, 575 aa, synthetic construct; identical to SEQ ID NO: 18
  REGION 1..575: note = pMYT1363_mature_protein

SEQ ID NO: 23 - DNA, 1548 bp, synthetic construct
  misc_feature 1..1548: note = pMYT1364_CDS_DNA
  source 1..1548: mol_type = other DNA
atgtacgccaagttcgcgaccctcgccgcccttgtggctggcgccgctgctcaggacgtcatcgaccgcatctacaagcaggtcgccctggagtcgccgctcgccctgctcaacaccgagtccgtcatcatgaacgccatcaccagcctgtcgtaccagatcaacggcgccgagaactcctcgggctgcggcgcccctgtccacgaccccgactacatcggcggcatcggcaaggagctcatcgtcgacgacatctccgacgtcaccagcttctacccctcggcctaccaggagcacctgaacttcatcccggcccccaccaccggctcgggctgcacccgcatcccgtccttcgacatgagcaccacccactactgctacacccacaacgtcatcctctcgggctgccgcgaccactcgcactcgcaccagtacctggccctgggcgtcctgcgcacctcggccaccggccgcgtcttcttctcgaccctgcgctccatcaacctcgacgacacccagaaccgcaagtcctgctcggtctcggccaccccgctcggctgcgacatgctctgctccaaggtcaccggcaccgaggaagaggactacaagtcggtcgcccccacctccatggtccacggccgcctgggcttcgacggccagtaccacgagaaggacctggacaccaccgtcctcttcaaggactgggtcgccaactacccgggcgtcggcggcggcagcttcatcgacggccgcgtctggttccccgtctacggtggcctgaagccgaactccccctcggacaccgcccaggaaggcaagtacgtcatctacaagcgccacaacaacacctgccccgacaagcaggactaccagatccgcatggccaagtcgtcctacaagcccgcccgcttcggtggcaagcgcgtccagcaggccatcctctcgatcaaggtctccacctcgctgggccgcgacccggtcctcaccatcccgcccaacaccatcaccctgatgggcgccgagggccgcatcctcaccgtcggcaccagccacttcctgtaccagcgcggcagctcctacttctcgcccgccctgctctacccgatgaccgtcaacaacaagaccgccaccctccacagcccctacatgttcaacgccttcaccaggcccggctccgtcccctgccaggcctcggcccgctgccccaacagctgcatcaccggcgtctacaccgacccctacccgctcatcttctaccgcaaccacaccctccgcggcgtcttcggcaccatgctcgacgacgagcaggcccgcctcaaccccgtctccgccgtcttcgacaacatctcccgcagccgcgtcacccgcgtctccagctcctccaccaaggccgcctacaccacctccacctgcttcaaggtcgtcaagaccaacaaggcctactgcctctccatcgccgagatcagcaacaccctgttcggcgagttccgcatcgtccccctgctcgtcgagatcctcaaggacgaccgcgtcgagccggaggcctaa

SEQ ID NO: 24 - protein, 515 aa, synthetic construct
  REGION 1..515: note = pMYT1364_amino_acid
MYAKFATLAALVAGAAAQDVIDRIYKQVALESPLALLNTESVIMNAITSLSYQINGAENSSGCGAPVHDPDYIGGIGKELIVDDISDVTSFYPSAYQEHLNFIPAPTTGSGCTRIPSFDMSTTHYCYTHNVILSGCRDHSHSHQYLALGVLRTSATGRVFFSTLRSINLDDTQNRKSCSVSATPLGCDMLCSKVTGTEEEDYKSVAPTSMVHGRLGFDGQYHEKDLDTTVLFKDWVANYPGVGGGSFIDGRVWFPVYGGLKPNSPSDTAQEGKYVIYKRHNNTCPDKQDYQIRMAKSSYKPARFGGKRVQQAILSIKVSTSLGRDPVLTIPPNTITLMGAEGRILTVGTSHFLYQRGSSYFSPALLYPMTVNNKTATLHSPYMFNAFTRPGSVPCQASARCPNSCITGVYTDPYPLIFYRNHTLRGVFGTMLDDEQARLNPVSAVFDNISRSRVTRVSSSSTKAAYTTSTCFKVVKTNKAYCLSIAEISNTLFGEFRIVPLLVEILKDDRVEPEA

SEQ ID NO: 25 - protein, 498 aa, synthetic construct
  REGION 1..498: note = pMYT1364_mature_protein
QDVIDRIYKQVALESPLALLNTESVIMNAITSLSYQINGAENSSGCGAPVHDPDYIGGIGKELIVDDISDVTSFYPSAYQEHLNFIPAPTTGSGCTRIPSFDMSTTHYCYTHNVILSGCRDHSHSHQYLALGVLRTSATGRVFFSTLRSINLDDTQNRKSCSVSATPLGCDMLCSKVTGTEEEDYKSVAPTSMVHGRLGFDGQYHEKDLDTTVLFKDWVANYPGVGGGSFIDGRVWFPVYGGLKPNSPSDTAQEGKYVIYKRHNNTCPDKQDYQIRMAKSSYKPARFGGKRVQQAILSIKVSTSLGRDPVLTIPPNTITLMGAEGRILTVGTSHFLYQRGSSYFSPALLYPMTVNNKTATLHSPYMFNAFTRPGSVPCQASARCPNSCITGVYTDPYPLIFYRNHTLRGVFGTMLDDEQARLNPVSAVFDNISRSRVTRVSSSSTKAAYTTSTCFKVVKTNKAYCLSIAEISNTLFGEFRIVPLLVEILKDDRVEPEA

SEQ ID NO: 26 - DNA, 1410 bp, synthetic construct
  misc_feature 1..1410: note = pMYT1365_CDS_DNA
  source 1..1410: mol_type = other DNA
atgtacgccaagttcgcgaccctcgccgcccttgtggctggcgccgctgctggcgcccctgtccacgaccccgactacatcggcggcatcggcaaggagctcatcgtcgacgacatctccgacgtcaccagcttctacccctcggcctaccaggagcacctgaacttcatcccggcccccaccaccggctcgggctgcacccgcatcccgtccttcgacatgagcaccacccactactgctacacccacaacgtcatcctctcgggctgccgcgaccactcgcactcgcaccagtacctggccctgggcgtcctgcgcacctcggccaccggccgcgtcttcttctcgaccctgcgctccatcaacctcgacgacacccagaaccgcaagtcctgctcggtctcggccaccccgctcggctgcgacatgctctgctccaaggtcaccggcaccgaggaagaggactacaagtcggtcgcccccacctccatggtccacggccgcctgggcttcgacggccagtaccacgagaaggacctggacaccaccgtcctcttcaaggactgggtcgccaactacccgggcgtcggcggcggcagcttcatcgacggccgcgtctggttccccgtctacggtggcctgaagccgaactccccctcggacaccgcccaggaaggcaagtacgtcatctacaagcgccacaacaacacctgccccgacaagcaggactaccagatccgcatggccaagtcgtcctacaagcccgcccgcttcggtggcaagcgcgtccagcaggccatcctctcgatcaaggtctccacctcgctgggccgcgacccggtcctcaccatcccgcccaacaccatcaccctgatgggcgccgagggccgcatcctcaccgtcggcaccagccacttcctgtaccagcgcggcagctcctacttctcgcccgccctgctctacccgatgaccgtcaacaacaagaccgccaccctccacagcccctacatgttcaacgccttcaccaggcccggctccgtcccctgccaggcctcggcccgctgccccaacagctgcatcaccggcgtctacaccgacccctacccgctcatcttctaccgcaaccacaccctccgcggcgtcttcggcaccatgctcgacgacgagcaggcccgcctcaaccccgtctccgccgtcttcgacaacatctcccgcagccgcgtcacccgcgtctccagctcctccaccaaggccgcctacaccacctccacctgcttcaaggtcgtcaagaccaacaaggcctactgcctctccatcgccgagatcagcaacaccctgttcggcgagttccgcatcgtccccctgctcgtcgagatcctcaaggacgaccgcgtcgagccggaggcctaa

SEQ ID NO: 27 - protein, 469 aa, synthetic construct
  REGION 1..469: note = pMYT1365_amino acid
MYAKFATLAALVAGAAAGAPVHDPDYIGGIGKELIVDDISDVTSFYPSAYQEHLNFIPAPTTGSGCTRIPSFDMSTTHYCYTHNVILSGCRDHSHSHQYLALGVLRTSATGRVFFSTLRSINLDDTQNRKSCSVSATPLGCDMLCSKVTGTEEEDYKSVAPTSMVHGRLGFDGQYHEKDLDTTVLFKDWVANYPGVGGGSFIDGRVWFPVYGGLKPNSPSDTAQEGKYVIYKRHNNTCPDKQDYQIRMAKSSYKPARFGGKRVQQAILSIKVSTSLGRDPVLTIPPNTITLMGAEGRILTVGTSHFLYQRGSSYFSPALLYPMTVNNKTATLHSPYMFNAFTRPGSVPCQASARCPNSCITGVYTDPYPLIFYRNHTLRGVFGTMLDDEQARLNPVSAVFDNISRSRVTRVSSSSTKAAYTTSTCFKVVKTNKAYCLSIAEISNTLFGEFRIVPLLVEILKDDRVEPEA

SEQ ID NO: 28 - protein, 452 aa, synthetic construct
  REGION 1..452: note = pMYT1365_mature_protein
GAPVHDPDYIGGIGKELIVDDISDVTSFYPSAYQEHLNFIPAPTTGSGCTRIPSFDMSTTHYCYTHNVILSGCRDHSHSHQYLALGVLRTSATGRVFFSTLRSINLDDTQNRKSCSVSATPLGCDMLCSKVTGTEEEDYKSVAPTSMVHGRLGFDGQYHEKDLDTTVLFKDWVANYPGVGGGSFIDGRVWFPVYGGLKPNSPSDTAQEGKYVIYKRHNNTCPDKQDYQIRMAKSSYKPARFGGKRVQQAILSIKVSTSLGRDPVLTIPPNTITLMGAEGRILTVGTSHFLYQRGSSYFSPALLYPMTVNNKTATLHSPYMFNAFTRPGSVPCQASARCPNSCITGVYTDPYPLIFYRNHTLRGVFGTMLDDEQARLNPVSAVFDNISRSRVTRVSSSSTKAAYTTSTCFKVVKTNKAYCLSIAEISNTLFGEFRIVPLLVEILKDDRVEPEA

SEQ ID NO: 29 - DNA, 13412 bp, synthetic construct
  misc_feature 1..13412: note = pMYT1055
  source 1..13412: mol_type = other DNA
cgcgccttatcggacggcctgatttgcggcatgcctcatcgcggcgaaactgctgataagacctgctgccgcagtccgcccattcctgtgtctgacgcacaccgattggggcatgtccgcggaaaaagagacgatcctctccactaggtggtttccgcctttaacggatcgaggaaacattagttgttagtagtaacacgccttgaacgccttgatctccggggctcctcctcggacgaccgcggaccgccggtcagagtgggtgggtacaccacactacacgatactatcgcgcatcgtcgttgtccctgctctctccccatatggcgtcacggggtagtatatattctacacggagctggagccaaggcaggcccaccgtacggattacaccgccatggttccaagtttcttcgccattgaatctgttattcgtgtactagtagatcaagccattgccggttgccgatatcatgcaactccggtcaggccacggagaagccatgcccgctacgggttgatgttccctgttccctttcataaacgggaataagacgaagtgcgcggcgggagttgggagcccatacccgacctcgatgctttccccggttcttccattccctagtactagtgtagctatttcagagtcttcatgccagcccttccccggaggcccctggcgcccccccaccccttcctctcctggcgagagtgcacacagtctttttgtacagtcttggtcccatctagtgggcgttttccccttctttcgtccccattgtccgtagagagagtgttgggtccgaccgcctaccgacgacaatggagatggcgccctcgacgacctcctcctcttcttcccttgaagggtccccgatgaccttcccatcacacctgccgccaacccctccgctgcaggattttgacgaatatcagaaactaggtctcagcctgccgacagatggcctcggacgatccgtccctccgtaccctctccctccgccgtcgaggcagaacaaggtgctgcccgaggacctcaaaacgccggatgaccacgtggagcgggacccccgcctgatccggctcaccggagtccaccccttcaacgtcgagccccccctgaccgacctctttgacgaaggcttcctgaccaccaaggacctgcactacgtccgcaaccatggcgccgtcccccgggtcgaggactcatccgtgctcgactgggacttcaccgtcgagggcatggtcgcccacccgatcacgctcacgctgcgcgacctgatccggacgtacgaccaggtgacgtacccggtcaccctggtgtgcgcgggcaaccggcgcaaggagcagaacgtggtgcgcaagaccaagggtttctcgtggggtgccgccggcctgtcgacggcgctgtggacgggcgtcgccctgcccgacctcctcgcccgcgccgccccgctgcgcggtgcgcggtacgtctgcttcgagggcgccgaccgcctgcccaacggctgctacggcacctcggtccgcctcggctgggccacggacccggcgcgtggcatcatgctcgcccaccgcatgaacggcgagccgctgcccccggaccacggcaagcccctccgcgtcatcatcccgggccagatcggcggcaggagcgtcaagtggctcaagcgcatcatcgtcacggcgaagcccagcgacagctggtaccacatctacgacaaccgcgtgctgcccaccgtgatcagccccgaggagagcgccgaccggcccgacgtctggaaggacgagcggtacgccatctacgacctcaacaccaacagcgccatctgctatccggcgcacgacgagagggtgccgctcgggatcgcgcgcggggacgcggcgacgtacaagctgcgggggtacgcctacgccggcggcggcaagagggtgacccgtctggaggtgacgctcgaccagggccggacgtggatgctggcgaccatgtcgtacccggaagacctgtaccggctcgcgcccgagggggagacgctgtttggcgggaggctggacgtgtcctggcgcgagacgtgcttctgctggtgcttctggtcgctggacgtgccgctgtcgcggctgcgagacgcccgcgacgtcatgatccgggcgatggacgagtccatgatggtgcagccgcgggacatgtactggagcgtgctgggcatgatgaacaacccatggtaccgggtcgtcatccacaaggagagcgacacgttgcgattcgagcaccccacccagccggcgctgatgcccggcggttggatggagcgggtcaagaaagaagggggcaaccttaccaatgggttttggggcgagaaggtgggcggccaggacgcggcgccggccgacgaggctcccgccgccaaagagatcagcatggtaaacgagaaggtcacccgccgcatcacccccgaagaactccgccagcactcgggcgaggatgagccgtggttcgtcgtcaacggccaggtctatgacggcaccaagttcctcgaaggccatcccggcggcgccgcctccatcatcaacgccgcgggacaagacgtgagcgaggagttcctcgcgattcgtaagttgccctccctccttcctttcttccccccctcccaccccctctcccttcttcccttccaaatttatctcggcaaccaaccctgacacggccaaaaaaaaaaaaaaaaaaacgaaacacagacagcgagaacgccaaggccatgatgccgagttaccacattggcacgctgccggcctcggccgacctctccgaggagccctcggacgaggcggtctcctcctcctccgccgtcttcctccaacccaaaacctggcgccgcgccctcctcgcctccaagacggccgtatcaccggacaccaaggtcttccacttcaccctgccctcgccggcgcaggccgtcggcctcccggtgggccagcacctcctcgtccgcctgcgcgacccggtcacgcgcgaggccatcatccgcgcctacacgccgctgtccgagggcggcgaggtgggcaccctgcgcgtgctcgtcaagatctaccgcgactcgcccgacgggtcgcgcaagggcggccgcatgacgcaggcgctcgactccctgcccgtcggccaccccgtcgagttcaagggccccgtgggcaagttcgagtacctgggcgcggggaggtgcgtcgtcggcggccgggagcggacggtcgcccggttcgtcatggtctgcggcgggtccggcgtgacccccatctttgccgtcctccgggccgtggcgaggggcgagcgggacgagacggcgtgtctggtgctcgacggcaaccgcggggaaggggacatcttgctgcgcgaggaactggatgggctggtgcgggagagccgggggaggtgtaggattgtgcacgctctgagcaggccgagagacgggtggaaggggttgaaggggcggatggatagggccttcttcgagagggaggttggcgctccgccggctgaaaggaacgcgatggtattaatttgcgggcccgaaggcatggagaaggcagcgagggaagcattcttggggatgggctgggatgaggacgatctccttttcttttagtttagcatcatgacatgaactttggaagggcgacgtggcgttggcaggcgtcgggaaaatgcatggtcccggtattcttgtttttgattctaagagggagggggggaagggggagagaaatgtaaagaaaagggcacagccatgattcatgattctcaaatgaccgaccactctggcgaactcgaggggccatgaaagagagtcctcaaggttgaaccgcacatcaaccgaggtggggtgccgtgaaggaagggtgaagtggcgctgcagaactgatttccccggaggtactcaatctttaggcggagacttccagtttagcttgtttattagcccagctgcttgcatctccggcgctgtcagccgagaatggaaggcaccgtgtaacgcagagcacgatttggtgtatttcatccgtataattaactagggaaaagtgagctggaaatacgcaggtactgtacggattaccttcgttgcacaatttaaattctccttagctctgtacagtgaccggtgactctttctggcatgcggagagacggacggacgcagagagaagggctgagtaataagcgccactgcgccagacagctctggcggctctgaggtgcagtggatgattattaatcagggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtctgtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacggtttaaaccgccggcgggttgccacggtcacggaatgatggagcgggtctgacacaccaaacactcgataagcagagcggcgatgtccatgctccggaacagccagcccgagttgccgtcgcccatagtgttccaagtaagcgtatgggacgagaagaaagaagagaaaagaagaagagagccgcccggctcgttcagagtcggccgcacggttcggtttcctgggagcgaagggacgtgaatcgccagagcgaccgaggtttagccacggcaagaaagcaaaatccggagttttgccagagagggcgcgcatcgctaaagtgctccttggcccggatgcgtctcgtctcgcctcagagagatggataccccgtgccttggctagaaagcctggcttgcgagatgctcctctcggcgcgggcggcgtggtgccgtggttcgtttgctgagctagaacgacgcccctgcgcgtgaccagcgtgtttttggtttgttcacccccagaatcagccgaggtttacccagccggagcgctcgtcaggtccttcacgggccgtggatccggaaactatgcgaaggtggagagagcgactcggagcgactttcgcgattattctggcgagttttcgagggcgcggtaactgcgaaagctgaccttcttcaattcaatcaacaatggaaggagaggtcgaattcggaggtttaccccccccccgcgccaaaatagtctctcccaggtgcccatgtaaggcacgggaatacctgcctaacttggggttgggaaagaaaagaaaggcaggaaaaaaatacactatcataccatgctcaagctttctctttccagaaacatgtgtttcgggttctccttccgtgctcccagagctggccctttggcccgttgggggttggcggggcccaagggtgcgctagtggggctcctcagtctccgcaatatcgtgcatcgcaacgcgcaaggcccttgcccactatcaacagccccccggattgctgaccgttggccattcacggcccttcgtttgccagtccttcgccagggtcaacctaccccgcggtggggggttgttcttggatccttgtcgaggccccggctgcccacatcgcccacgctgtgcactcagcgtaacacagggcccggatctctcaagcgatgcccagctttttttcatcggtgttgacggtccgaaactcgcgggagagatgggggcagatcatggcgggaaacggccgtgatggttcctggatataaaggagatcaggccttccctcctcggctcattggggcctactagcacatcatcatccgtcttccatccctcctcagaacttccttccccttcctcctatccacctttcccttactcacacagacaatcgtccatcgtccaccttaattaaggccctatgacctcccgagtcctggtcgagagaggggagctggagagagagagagagagagagagggagcatgaagccgtactgattcggcaaaccattttgaagcctggacatgtttaccttcgttttctcttcttggatggtttgcattattattagccgcttttcttttttctctccattcgttatacatattttttgttttgttttcctcttgttgttcattgtttggccctgggccttttttcttcttctcatttctcttttcacttcttcttcttcttcttcttcttcttcttcttcctcctcctcctcctccttcgctttgcgggaccgcttcttggggctcgggaggaccagtggtatgcttgcgagcacacacatcgatgcatggtgtttctttcatgttgtacctatcgttcagtgttttgcccccggtttgggcatttcattcgcttcgcttgtacatacacatggacaatacattcttcgtatggcttcaggctagacatctcccctaccaaccacacctgcaccatgcccgtcttgtcaacctacctaacttacataccgatgttcgccggccgcgagctgctgcagaggtttgtgacggagacacacatcgatttctcacctgcccgtgg

SEQ ID NO: 30 - protein, 364 aa, Newcastle disease virus
MDSSRTIGLYFDFALPSSSLLAFPIVLQDTGDGKKQITPQYRIQRLDSWTDSKEDSVFIITYGFIFQIGNEEATVGVINDNPRHELLSSAMLCLGSVPNDGDLVELARACLTMVVTCKKSATNTERIVFSVVQAPRVLQSCMVVANRYSSVNAVKHVKAPEKIPGSGTLEYKVNFVSLTVVPRRDVYRIPTAVLKVSGSSLYNLALNVTIDVDVDPKSPLVKSLSKSDSGYYANLFLHIGLMSTVDKKGKKVTFDKIEEKIRRLNLSVGLSDVLGPSVLVKARGARTKLLAPFFSSSGTACYPIANASPQVAKILWSQTAHLRSVKVIIQAGTQRAVAVTADHEVTSTKIERRHAIAKYNPFRK

SEQ ID NO: 31 - protein, 395 aa, Newcastle disease virus
MATFTDAEIDDIFETSGTVIDSIITAQGKSAETVGRSAIPQGKTKALSTAWEKHGSVQPHASQDAPDQQDRTEKQPSTPEQATPHNNPPITSTEPPPTQAASETSDTQLKTGASNSLLSMLDKLSNKSSNAKKGPWSGPQEGHHQPPAQQHGNQPSYGSNQGRPQHQAKAVPGNRGTDENTAYHGQRKESQPSAGATPHAPQSGQSQDNTPVPVDRVQLPADFAQAMMSMMEALSQKVSKVDHQLDLVLKQTSSIPMMRSEIQQLKTSVAIMEANLGMMKILDPGCANVSSLSDLRAVARSHPVLVSGPGDPSPYMTQGGEMTLNKLSQPVQHPSELIKSATASGPDMGVEKDTVRALITSRPMHPSSSAKLLSKLDAARSIEEIRKIKRLALNG

SEQ ID NO: 32 - protein, 489 aa, Newcastle disease virus
MSSVFDEYEQLLAAQTRPNGTHGGGEKGSTLKVEVPVFTLNSDDPEDRWNFAVFCLRIAVSEDANKPLRQGALISLLCSHSQVMRNHVALAGKQNEATLAVLEIDGFANSVPQFNNRSGVSEERAQRFMVIAGSLPRACSNGTPFVTAGVEDDAPEDITDTLERILSIQVQVWVTVAKAMTAYETADESETRRINKYMQQGRVQKKYILHPVCRSAIQLTIRHSLAVRIFLVSELKRGRNTAGGSSTYYNLVGDVDSYIRNTGLTAFFLTLKYGINTKTSALALSSLTGDIQKMKQLMRLYRMKGENAPYMTLLGDSDQMSFAPAEYAQLYSFAMGMASVLDKGTGKYQFARDFMSTSFWRLGVEYAQAQGSSINEDMAAELKLTPAARRGLAAAAQRVSEEIGSMDIPTQQAGVLTGLSDEGPRTPQGGSNKPQGQPGAGDGETQFLDFMRAVANSMREAPNPAQSTTHPEPPPTPGASQDNDTDWGY

SEQ ID NO: 33 - DNA, 8756 bp, synthetic construct
  misc_feature 1..8756: note = pMYT1140_DNA
  source 1..8756: mol_type = other DNA
ggcgcgccgtttaaactcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcccctgggttcgcaaagataattgcactgtttcttccttgaactctcaagcctacaggacacacattcatcgtaggtataaacctcgaaaatcattcctactaagatgggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagaaggagaggcgcgccgcttttcttttttctctccattcgttatacatattttttgttttgttttcctcttgttgttcattgtttggccctgggccttttttcttcttctcatttctcttttcacttcttcttcttcttcttcttcttcttcttcttcctcctcctcctcctccttcgctttgcgggaccgcttcttggggctcgggaggaccagtggtatgcttgcgagcacacacatcgatgcatggtgtttctttcatgttgtacctatcgttcagtgttttgcccccggtttgggcatttcattcgcttcgcttgtacatacacatggacaatacattcttcgtatggcttcaggctagacatctcccctaccaaccacacctgcaccatgcccgtcttgtcaacctacctaacttacataccgatgttcgccggccgcgagctgctgcagaggtttgtgacggagacacacatcgatttctcacctgcccgtatttaaatgccctccgtccccttccttcacaacctcgaattcctcccaatgtggatatctgtcgcctttctaagaaagggcgtggaacacgcgcgattagtatagaatatggatcgacctaagttgtctccgcacatgtctcaacagtctagcgacaagaagaacctcgcccacccgtcgattacgagcgtgtgcagcctgagtgtgtgtgagttggagttaacggcgccgaaatctgaaggagggaagagacttttcaacacgtctgttctctactgactttttttgtttttaccacatcgcactaggaaagctagcggtgttttgatggtgccaatactgcgtaactgcgtaatgttgcatattgcgtagcagcgtgaagcaagtggatgtatgtacggactaatccgtatgcactgcatctcgcagcagatggcacctccccagagacagccgggaaacaagcttttttttccttggcgtccttggcttgcatggcttcattggcgggtgttatgttttccccaggggtgcagcaatggcacgccgagcaaaaaaggaaccgcggacctggcacaagccccaaactccatcgacgcaggacggcatcgcattgcgttgcgcctcctctccaacgacgtcttaagggaaaagaaaagaaaaacaaaaggataatggcaggcctccagcaagcaagcaagagcgcttccggccgttcaagggtccaatccggttcaagccttgcgttatcgtcccagagggcggcccttgttgtaagccggccttgtgttcgcgcccttcgatgtttgacattgcttttccgtctgggtactttccagtcggttgttggagacttcctcgtcattgtatggggtcacatgttctctgcgcactacactacggagtaagtgctaataaattacatctcgaccccgtgcttggcaacaacctcgaggaacctgtcctgcttgccttatttccgtcggttgggtagacggcttgttcgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 34 - DNA, 9814 bp, synthetic construct
  misc_feature 1..9814: note = pMYT1727_DNA
  source 1..9814: mol_type = other DNA
gtttaaacaaccgactaacgaccgttctattattattttttcttcttccccgccaggttcaatcttagcaacgccgctaccagcctacctccgggtgccgctgcgatactaccgcgcgctgacggtcccctaccacgacgacgagaccaacagccagatccaccgcgtctaccagccggcgggcgaggagcacgcaatgcggcacttctgcgggttctgcggcaccccgctctcgtactggtccgagtcgccgcgcagcgaggccgacttcatccgcctgaccctgggcagcctgctgcatgaagacctaagagacctggaggaatgggggttggtccccgatcccgactccgcctccggcacggggacgcctttggaacaagaagatgaggcggcggaaggaaaccggggcaaaagtggggaggggaagacgcgtacggacgctgcgacgggagctgagggagaaagggaggtcggggaagcgggggggaatgtttggggcagggtcggggtgctgccgtggttcgagaccctcacggagggcagccggctagggacgaccttgcgacgggcgagaggtggggggacagacccaacggaaagggtgaggattgagtgggagattgctgagtggagtgccgaggacgaaaagggtaatgagagtccacgtaagcggaagttggatgaggttgaggacgccgttgaggccgagaggacggtgggcgtgcgtgtacaatagagtgatgtggttgcctcgcatgcaagacggcaaacgcacacccgtgccatgcatgccacgggtaaggggtgaggagattggtctgcgtggggggcatataagaccttaatttagggctctctatgatatcgaccggcaagaatcctggacatctcactcgctacaaggtgcgcttgcttcttggacgcagctagctgatgatgtttcgcatcttcaacctgcctccaaacagcgacaatgcagttgcatcttcgtgtagaagagccgcgcggttaatcttcaatccaccgagtacggtagatcaatccaggcaataactactgcgccggcgagattcatcaactcattgctggagttagcatatctacaattgggtgaaatggggagcgatttgcaggcatttgctcggctagtcggaatgaacattcattccgagacctaggatgtgacggaatgaaggttcattccggactctagataagcacggaatgaactttcattccgctgaagcttgtcaatcggaatgaaggttcattccggctagtcggaatgaacattcattccgagacctaggatgtgacggaatgaaggttcattccggactctagataagcacggaatgaactttcattccgctgaagcttgtcaatcggaatgaaggttcattccggctagttctccccggaaactgtggccatatgttcaaagactaggatggataaatggggtatataaagcaccctgactcccttcctccaagttctatctaaccagccatcctacactctacatatccacaccaatctactacaattacaaccatattaattaaggccctatgacctcccgagtcctggtcgagagaggggagctggagagagagagagagagagagagagagggagcatgaagccgtactgattcggcaaaccattttgaagcctggacatgtttaccttcgttttctcttcttggatggtttgcattattattagccgcttttcttttttctctccattcgttatacatattttttgttttgttttcctcttgttgttcattgtttggccctgggccttttttcttcttctcatttctcttttcacttcttcttcttcttcttcttcttcttcttcttcctcctcctcctcctccttcgctttgcgggaccgcttcttggggctcgggaggaccagtggtatgcttgcgagcacacacatcgatgcatggtgtttctttcatgttgtacctatcgttcagtgttttgcccccggtttgggcatttcattcgcttcgcttgtacatacacatggacaatacattcttcgtatggcttcaggctagacatctcccctaccaaccacacctgcaccatgcccgtcttgtcaacctacctaacttacataccgatgttcgccggccgcgagctgctgcagaggtttgtgacggagacacacatcgatttctcacctgcccgtggcgcgccctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcggtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 35 - DNA, 10424 bp, synthetic construct
  misc_feature 1..10424: note = pMYT1729_DNA
  source 1..10424: mol_type = other DNA
ggcgcgccgtttaaaccccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtatttaaatctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctatttaaatgagcgcgaatgaacactacgttggagaagaactcgacacatccgacaagacctcacatacccagacctgcccaggtggtatttgatatcaggatcaatggcgtcggacattgctacagcactcttgacgcctgtgaccgaatcgctccagcgctacaaattacccagtagccgggcactaacagctccctggcctaggtagactacctacctcaaggtacgacacatggcagcactggagggggaataggcagactggacgacagtggacaagatacggtcgcacaacctttgtcgtggcatcgcgagaataatcgtcacaagcttcacgtatgcagacggagacaagatgatttggttgtcgaagtcatgaattcacttctatctagtttttttgttcccttttgttttgcattcccagagaagttctgatggaacccttattcccagcctctcaattaacgtgcctcgattcatagtcgagtgctcatgcatagcaacattgatcgtttcgtcgtagaagtgagcgcatggtggtgcccacctggagaaacctcacgagggaccccagaacatcaggtgttgatgatgggtatcgcggccggcctcagccgccgtactcgtcaatgcccagggcgtcggtgaacatctgctcaaactcgaagtcggccatgtccagggcgccgtagggggcggagtcgtggggggtaaagccggggccggggctgtcgccgtcgcccagcatgtcgaggtcgaagtcgtcgagggcgtcggcgtgggccatggcgacgtcctcgccgtccaggtggagctcgtcgccgagggagacgtccgtggggggggccgtgctgaccttgcgcttcttcttgggagggctagccgattgtcgggagagagcagcccagagcgattcctctacccccgtaagcaactcatccgttagagagagataatcgttttcgatcatctcatagacctccataaacgatccgaataggatggcgatcagggcattctcgggcaagtttcgaattacgccctgtttctgtccctctcgaaagaaggtgcagacgaactcaacaagtttttggtatgcaaggcgtgactcttcggttaggaatgtaccttgggaatgtgtcttgataaatcccaaggcgcgcggatggttctttgtgaatgtgaccattccctcgaagatatgatggaacccatcgcgataaccgtccctttcgttcgccaagccactctcgatacattgcaaaaattcattaacgtgctgctggaacagctcgttgaccagactctccttattcttaaagtatcggtaaatcgttcctgcgccgaccttagcattttcagcgatcatcggcatcgtagtggcgtcaaacccgcgttcggcgaacagaaggagcgaggcagaaaaaatagctttttgtttcgtgggtgtggactccattgttaatttttttcaaatatgaatttgattgatgggagagggaaaataaaagaagaagaaaaccgaatagaaagaaggtagagagaaaacgaagaatcgaccaagtgggggcgggggggaggagggaaagtggttgatttatagtttggaattcagggcgagaccaattactacagacttattactttgggttcgccggcgaatgccctccgtccccttccttcacaacctcgaattcctcccaatgtggatatctgtcgcctttctaagaaagggcgtggaacacgcgcgattagtatagaatatggatcgacctaagttgtctccgcacatgtctcaacagtctagcgacaagaagaacctcgcccacccgtcgattacgagcgtgtgcagcctgagtgtgtgtgagttggagttaacggcgccgaaatctgaaggagggaagagacttttcaacacgtctgttctctactgactttttttgtttttaccacatcgcactaggaaagctagcggtgttttgatggtgccaatactgcgtaactgcgtaatgttgcatattgcgtagcagcgtgaagcaagtggatgtatgtacggactaatccgtatgcactgcatctcgcagcagatggcacctccccagagacagccgggaaacaagcttttttttccttggcgtccttggcttgcatggcttcattggcgggtgttatgttttccccaggggtgcagcaatggcacgccgagcaaaaaaggaaccgcggacctggcacaagccccaaactccatcgacgcaggacggcatcgcattgcgttgcgcctcctctccaacgacgtcttaagggaaaagaaaagaaaaacaaaaggataatggcaggcctccagcaagcaagcaagagcgcttccggccgttcaagggtccaatccggttcaagccttgcgttatcgtcccagagggcggcccttgttgtaagccggccttgtgttcgcgcccttcgatgtttgacattgcttttccgtctgggtactttccagtcggttgttggagacttcctcgtcattgtatggggtcacatgttctctgcgcactacactacggagtaagtgctaataaattacatctcgaccccgtgcttggcaacaacctcgaggaacctgtcctgcttgccttatttccgtcggttgggtagacggcttgttcgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 36 - DNA, 9655 bp, synthetic construct
  misc_feature 1..9655: note = pMYT1730_DNA
  source 1..9655: mol_type = other DNA
cgcgccctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcggtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacggtttaaaccgccggcgggttgccacggtcacggaatgatggagcgggtctgacacaccaaacactcgataagcagagcggcgatgtccatgctccggaacagccagcccgagttgccgtcgcccatagtgttccaagtaagcgtatgggacgagaagaaagaagagaaaagaagaagagagccgcccggctcgttcagagtcggccgcacggttcggtttcctgggagcgaagggacgtgaatcgccagagcgaccgaggtttagccacggcaagaaagcaaaatccggagttttgccagagagggcgcgcatcgctaaagtgctccttggcccggatgcgtctcgtctcgcctcagagagatggataccccgtgccttggctagaaagcctggcttgcgagatgctcctctcggcgcgggcggcgtggtgccgtggttcgtttgctgagctagaacgacgcccctgcgcgtgaccagcgtgtttttggtttgttcacccccagaatcagccgaggtttacccagccggagcgctcgtcaggtccttcacgggccgtggatccggaaactatgcgaaggtggagagagcgactcggagcgactttcgcgattattctggcgagttttcgagggcgcggtaactgcgaaagctgaccttcttcaattcaatcaacaatggaaggagaggtcgaattcggaggtttaccccccccccgcgccaaaatagtctctcccaggtgcccatgtaaggcacgggaatacctgcctaacttggggttgggaaagaaaagaaaggcaggaaaaaaatacactatcataccatgctcaagctttctctttccagaaacatgtgtttcgggttctccttccgtgctcccagagctggccctttggcccgttgggggttggcggggcccaagggtgcgctagtggggctcctcagtctccgcaatatcgtgcatcgcaacgcgcaaggcccttgcccactatcaacagccccccggattgctgaccgttggccattcacggcccttcgtttgccagtccttcgccagggtcaacctaccccgcggtggggggttgttcttggatccttgtcgaggccccggctgcccacatcgcccacgctgtgcactcagcgtaacacagggcccggatctctcaagcgatgcccagctttttttcatcggtgttgacggtccgaaactcgcgggagagatgggggcagatcatggcgggaaacggccgtgatggttcctggatataaaggagatcaggccttccctcctcggctcattggggcctactagcacatcatcatccgtcttccatccctcctcagaacttccttccccttcctcctatccacctttcccttactcacacagacaatcgtccatcgtccaccttaattaaggccctatgacctcccgagtcctggtcgagagaggggagctggagagagagagagagagagagagggagcatgaagccgtactgattcggcaaaccattttgaagcctggacatgtttaccttcgttttctcttcttggatggtttgcattattattagccgcttttcttttttctctccattcgttatacatattttttgttttgttttcctcttgttgttcattgtttggccctgggccttttttcttcttctcatttctcttttcacttcttcttcttcttcttcttcttcttcttcttcctcctcctcctcctccttcgctttgcgggaccgcttcttggggctcgggaggaccagtggtatgcttgcgagcacacacatcgatgcatggtgtttctttcatgttgtacctatcgttcagtgttttgcccccggtttgggcatttcattcgcttcgcttgtacatacacatggacaatacattcttcgtatggcttcaggctagacatctcccctaccaaccacacctgcaccatgcccgtcttgtcaacctacctaacttacataccgatgttcgccggccgcgagctgctgcagaggtttgtgacggagacacacatcgatttctcacctgcccgtgg

SEQ ID NO: 37 - DNA, 10639 bp, synthetic construct
  misc_feature 1..10639: note = pMYT1731_DNA
  source 1..10639: mol_type = other DNA
cgcgccgtttaaaccccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtatttaaatctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctatttaaatgacgtgtatcataagttaagtaggtactgtacgttagtttgtttaccttcttcagccagtagtccgactttgctctcaagtgctcatccaaccctttggccttccaaatcttgataccgagagcctcaggcaggttggtatattctacctcaaatacctgccagatgttgaccaatgccgttgtcgttactccgtactacgcgaaccaatgccaacatgattctcctttcagtcacgagacgattcgagtgtcatggtagcagtactttggtagtaaagagtcactcaattgaacatgtcgtagctcattcgtaaccaagtcatgataaccctgaatttcaggggttcccttcagagacaggagccgtcatgttgcggcaagagaaaaaaaaatgaagaggaaacacacaaaaacgtgggaagaatagccatcagcggctaccctcatactccaactcctgcctgccttaattaaggtggacgatggacgattgtctgtgtgagtaagggaaaggtggataggaggaaggggaaggaagttctgaggagggatggaagacggatgatgatgtgctagtaggccccaatgagccgaggagggaaggcctgatctcctttatatccaggaaccatcacggccgtttcccgccatgatctgcccccatctctcccgcgagtttcggaccgtcaacaccgatgaaaaaaagctgggcatcgcttgagagatccgggccctgtgttacgctgagtgcacagcgtgggcgatgtgggcagccggggcctcgacaaggatccaagaacaaccccccaccgcggggtaggttgaccctggcgaaggactggcaaacgaagggccgtgaatggccaacggtcagcaatccggggggctgttgatagtgggcaagggccttgcgcgttgcgatgcacgatattgcggagactgaggagccccactagcgcacccttgggccccgccaacccccaacgggccaaagggccagctctgggagcacggaaggagaacccgaaacacatgtttctggaaagagaaagcttgagcatggtatgatagtgtatttttttcctgcctttcttttctttcccaaccccaagttaggcaggtattcccgtgccttacatgggcacctgggagagactattttggcgcggggggggggtaaacctccgaattcgacctctccttccattgttgattgaattgaagaaggtcagctttcgcagttaccgcgccctcgaaaactcgccagaataatcgcgaaagtcgctccgagtcgctctctccaccttcgcatagtttccggatccacggcccgtgaaggacctgacgagcgctccggctgggtaaacctcggctgattctgggggtgaacaaaccaaaaacacgctggtcacgcgcaggggcgtcgttctagctcagcaaacgaaccacggcaccacgccgcccgcgccgagaggagcatctcgcaagccaggctttctagccaaggcacggggtatccatctctctgaggcgagacgagacgcatccgggccaaggagcactttagcgatgcgcgccctctctggcaaaactccggattttgctttcttgccgtggctaaacctcggtcgctctggcgattcacgtcccttcgctcccaggaaaccgaaccgtgcggccgactctgaacgagccgggcggctctcttcttcttttctcttctttcttctcgtcccatacgcttacttggaacactatgggcgacggcaactcgggctggctgttccggagcatggacatcgccgctctgcttatcgagtgtttggtgtgtcagacccgctccatcattccgtgaccgtggcaacccgggaatgccctccgtccccttccttcacaacctcgaattcctcccaatgtggatatctgtcgcctttctaagaaagggcgtggaacacgcgcgattagtatagaatatggatcgacctaagttgtctccgcacatgtctcaacagtctagcgacaagaagaacctcgcccacccgtcgattacgagcgtgtgcagcctgagtgtgtgtgagttggagttaacggcgccgaaatctgaaggagggaagagacttttcaacacgtctgttctctactgactttttttgtttttaccacatcgcactaggaaagctagcggtgttttgatggtgccaatactgcgtaactgcgtaatgttgcatattgcgtagcagcgtgaagcaagtggatgtatgtacggactaatccgtatgcactgcatctcgcagcagatggcacctccccagagacagccgggaaacaagcttttttttccttggcgtccttggcttgcatggcttcattggcgggtgttatgttttccccaggggtgcagcaatggcacgccgagcaaaaaaggaaccgcggacctggcacaagccccaaactccatcgacgcaggacggcatcgcattgcgttgcgcctcctctccaacgacgtcttaagggaaaagaaaagaaaaacaaaaggataatggcaggcctccagcaagcaagcaagagcgcttccggccgttcaagggtccaatccggttcaagccttgcgttatcgtcccagagggcggcccttgttgtaagccggccttgtgttcgcgcccttcgatgtttgacattgcttttccgtctgggtactttccagtcggttgttggagacttcctcgtcattgtatggggtcacatgttctctgcgcactacactacggagtaagtgctaataaattacatctcgaccccgtgcttggcaacaacctcgaggaacctgtcctgcttgccttatttccgtcggttgggtagacggcttgttcgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacggg

SEQ ID NO: 38 - protein, 27 aa, synthetic construct
  REGION 1..27: note = Exemplary trimerization domain
GYIPEAPRDGQAYVRKDGEWVLLSTFL

SEQ ID NO: 39 - protein, 33 aa, synthetic construct
  REGION 1..33: note = Exemplary trimerization domain
RMKQLEDKVEELLSKVYHLENRVARLEKLVGER

SEQ ID NO: 40 - DNA, 1287 bp, synthetic construct
  misc_feature 1..1287: note = Exemplary antibody fragment
  source 1..1287: mol_type = other DNA
aagcttgcgagccccacatcgcccccccgattgtaccctctatccgcctgttgttccgactcggctgtcccgccggccgtgggctgcctgttgtccccttcgtccgccggcggcatctcctgggagggctccggaggtacggcggtggccggcagagtttcggggacccccgtgaagctcagcttcgtccgcctcagccccggcgagaagaggaaaagcttcgtctgcagcgccgcccccgggggggcgctgctcaaaaaggaggtgcaggtctgccgggtagatcccgtaccgcctgtagccccagaggtgcaggtcctccacccctcctcctgcaccccgagccaatccgagtcggtggagctgttgtgtttggtgacggggttctccccggcgtcggcggaggtcgaatggttggtggacggagtggggggacttttggtggcctcccaaagcccggcggtccgcagcggatccacctacagcctgagcagccgcgtcaacgtcagcggcaccgattggagggaagggaagagttacagctgtagggtgaggcaccccgcaaccaacaccgtggtggaggatcacgtcaagggatgcccggacggcgctcagagctgcagccccatccagctgtacgccatcccacccagcccgggcgagctgtacatcagcttagacgccaaactgaggtgcctggtggtcaacctgcccagcgattccagcctcagcgtcacctggaccagggagaagagtgggaacctccggcccgacccgatggtcctccaagaacacttcaacggcacctacagcgccagcagcgccgtccccgtcagcacccaggattggttatccggggagaggttcacctgcaccgtgcagcacgaggagctgcccctgccgctcagcaagagcgtctacaggaacacgggacccaccaccccacctctgatctaccccttcgccccccacccggaagagctgtccctctcccgcgtcaccctgagctgcctggtccgcggcttccgcccacgtgacatcgagatccggtggctccgcgaccaccgcgccgttcccgccaccgaattcgtcaccaccgccgtcctcccggaagagagaaccgcaaacggcgccggcggtgacggcgacaccttcttcgtgtacagtaagatgagcgtggagaccgccaagtggaacggcgggacggtgttcgcctgcatggcggtgcacgaggcgctgcccatgcgcttcagccagcgcacgctgcagaaacaggctggtaaataa

SEQ ID NO: 41 - protein, 428 aa, synthetic construct
  REGION 1..428: note = Exemplary antibody fragment
KLASPTSPPRLYPLSACCSDSAVPPAVGCLLSPSSAGGISWEGSGGTAVAGRVSGTPVKLSFVRLSPGEKRKSFVCSAAPGGALLKKEVQVCRVDPVPPVAPEVQVLHPSSCTPSQSESVELLCLVTGFSPASAEVEWLVDGVGGLLVASQSPAVRSGSTYSLSSRVNVSGTDWREGKSYSCRVRHPATNTVVEDHVKGCPDGAQSCSPIQLYAIPPSPGELYISLDAKLRCLVVNLPSDSSLSVTWTREKSGNLRPDPMVLQEHFNGTYSASSAVPVSTQDWLSGERFTCTVQHEELPLPLSKSVYRNTGPTTPPLIYPFAPHPEELSLSRVTLSCLVRGFRPRDIEIRWLRDHRAVPATEFVTTAVLPEERTANGAGGDGDTFFVYSKMSVETAKWNGGTVFACMAVHEALPMRFSQRTLQKQAGK

SEQ ID NO: 42 - DNA, 384 bp, synthetic construct
  misc_feature 1..384: note = Exemplary antibody fragment
  source 1..384: mol_type = other DNA
ggacagagtgcatgtgctgtacatacacagccatacatacgcgtgtggccgctctgcctctctcttgcaggccagcccaaggtggcccccaccatcaccctcttcccaccgtcaaaggaggagctgaacgaagccaccaaggccaccctggtgtgcctgataaacgacttctaccccagcccagtgactgtggattgggtgatcgatggctccacccgctctggcgagaccacagcaccacagcggcagagcaacagccagtatatggccagcagctacctgtcactgtctgccagcgactggtcaagccacgagacctacacctgcagggtcacacacaacggcacctctatcacgaagaccctgaagaggtccgagtgctaa

SEQ ID NO: 43 - protein, 127 aa, synthetic construct
  REGION 1..127: note = Exemplary antibody fragment
GQSACAVHTQPYIRVWPLCLSLAGQPKVAPTITLFPPSKEELNEATKATLVCLINDFYPSPVTVDWVIDGSTRSGETTAPQRQSNSQYMASSYLSLSASDWSSHETYTCRVTHNGTSITKTLKRSEC

(19) United States
(12) Patent Application Publication
(10) Pub. No.: US 2023/0372467 A1
(43) Pub. Date: Nov. 23, 2023

(54) USING FUNGAL CONSTRUCTS TO PRODUCE VIRAL PROTEIN ANTIGENS

(71) Applicants: Phibro Animal Health Corporation, Teaneck, NJ (US); VTT Technical Research Centre of Finland Ltd., Espoo (FI); Dyadic International (USA), Inc., Jupiter, FL (US)

(72) Inventors: Andriy Kovalchuk, Espoo (FI); Avi Zrachya, Airport City (IL); Avner Finger, Pardes Hanna-Karkur (IL); Noelia Valbuena, Jupiter, FL (US); Ronen Tchelet, Budapest (HU); Markku Saloheimo, Helsinki (FI)

(21) Appl. No.: 18/320,558

(22) Filed: May 19, 2023

Related U.S. Application Data
(60) Provisional application No. 63/344,252, filed on May 20, 2022.

(51) Int. Cl. A61K 39/17; C12N 15/80; C12N 1/14

(52) U.S. Cl. CPC A61K 39/17; C12N 15/80; C12N 1/145; C12N 2800/108; A61K 2039/5252

(57) ABSTRACT

The present disclosure concerns using transgenic fungus to express recombinant viral antigens. The composition, production, and administration of vaccines comprising those viral antigens also are disclosed. In some embodiments, these viral antigens can be used to formulate a vaccine against Newcastle Disease. These vaccines can be administered, for example, via intramuscular injection.

### CROSS REFERENCE TO RELATED APPLICATION

The present application claims the benefit of the earlier filing date of U.S. provisional application No. 63/344,252, filed on May 20, 2022, which is incorporated herein by reference in its entirety.

### SEQUENCE LISTING

The nucleic and amino acid sequences listed in the accompanying sequence listing are shown using standard letter abbreviations for nucleotide bases, and three letter code for amino acids, as defined in 37 C.F.R. § 1.822. Only one strand of each nucleic acid sequence is shown, but the complementary strand is understood as being included by any reference to the displayed strand. The Sequence Listing is submitted as an XML file in the form of the file named “10177-106793-02_ST26.xml” (˜139,557 bytes), which was created on May 11, 2023, and is incorporated by reference herein.

In the accompanying sequence listing:
SEQ ID NO: 1 is the amino acid sequence for the native C1 CBH1ss secretion signal.
SEQ ID NO: 2 is the amino acid sequence of a C-tag purification tag.
SEQ ID NO: 3 is the amino acid sequence of the NDV F protein from GenBank AZP53700.1 (F [Avian orthoavulavirus 1]).
SEQ ID NO: 4 is the amino acid sequence of the NDV Hemagglutinin-neuraminidase from GenBank AEU17158.1, Accession No. JQ013837.1, AA [Avian orthoavulavirus 1].
SEQ ID NO: 5 is the sequence for the nucleic acid which is part of the expression construct pMYT1358 and encodes the synthetic F protein of SEQ ID NO: 6, an amino acid sequence.
SEQ ID NO: 6 is the amino acid sequence translation of SEQ ID NO: 5 and contains a secretion signal.
SEQ ID NO: 7 is the amino acid sequence of SEQ ID NO: 6 wherein the secretion signal is cleaved.
SEQ ID NO: 8 is the sequence for the nucleic acid sequence which is part of the expression construct pMYT1359 and encodes the synthetic F protein of SEQ ID NO: 9.
SEQ ID NO: 9 is the amino acid sequence of the translated synthetic F protein of SEQ ID NO: 8 and contains a secretion signal.
SEQ ID NO: 10 is the mature protein of SEQ ID NO: 9 wherein the secretion signal is cleaved.
SEQ ID NO: 11 is the sequence contain the nucleic acid sequence which is part of the expression construct pMYT1360 and encodes for the amino acid sequence of SEQ ID NO: 12.
SEQ ID NO: 12 is the amino acid sequence for a synthetic NDV F protein translated from SEQ ID NO: 11.
SEQ ID NO: 13 is the mature protein of SEQ ID NO: 12 wherein the secretion signal is cleaved.
SEQ ID NO: 14 is the nucleic acid sequence for a synthetic NDV F protein of the expression constructs pMYT1361, pMYT1736, pMYT1737 and pMYT1738. It encodes for the amino acid of SEQ ID NO: 15.
SEQ ID NO: 15 is the amino acid sequence for a synthetic NDV F protein encoded by SEQ ID NO: 14 and contains a secretion signal.
SEQ ID NO: 16 is the amino acid sequence of mature protein of SEQ ID NO: 15 wherein the secretion signal is cleaved.
SEQ ID NO: 17 is the nucleic acid sequence for a synthetic NDV HN protein, which is a part of the expression construct pMYT1362. The translation of SEQ ID NO: 17 produces the amino acid sequence of SEQ ID NO: 18.
SEQ ID NO: 18 is the translated amino acid sequence of SEQ ID NO: 17 for a synthetic NDV HN protein and contains a secretion signal.
SEQ ID NO: 19 is the mature amino acid sequence of SEQ ID NO: 18 wherein the secretion signal is cleaved.
SEQ ID NO: 20 is the nucleic acid coding sequence which is part of the expression construct pMYT1363 and corresponds to the amino acid sequence of SEQ ID NO: 21 for a synthetic NDV HN protein.
SEQ ID NO: 21 is the amino acid sequence of SEQ ID NO: 20 and contains a secretion signal that, when cleaved, yields the mature protein of the SEQ ID NO: 22 amino acid sequence.
SEQ ID NO: 22 is the mature amino acid sequence of SEQ ID NO: 21 wherein the secretion signal is cleaved.
SEQ ID NO: 23 is the nucleic acid coding sequence for a synthetic NDV HN protein, which makes part of the expression construct pMYT1364.
SEQ ID NO: 24 is the amino acid sequence of SEQ ID NO: 23 for a synthetic NDV HN protein.
SEQ ID NO: 25 is the amino acid sequence of SEQ ID NO: 24 wherein the secretion signal is cleaved.
SEQ ID NO: 26 is the nucleic acid coding sequence for a synthetic NDV HN protein, which is part of the expression constructs pMYT1365, pMYT1741, pMYT1742 and pMYT1743. The translation of this sequence gives the amino acid sequence of SEQ ID NO: 27.
SEQ ID NO: 27 corresponds to a synthetic NDV HN protein which contains a secretion signal and is encoded by SEQ ID NO: 26.
SEQ ID NO: 28 is the amino acid sequence of SEQ ID NO: 27 wherein the secretion signal is cleaved.
SEQ ID NO: 29 contains the nucleic acid sequence for the C1 vector pMYT1055.
SEQ ID NO: 30 is the amino acid sequence of the NDV Matrix Protein [Avian orthoavulavirus 1] from NCBI Reference Sequence: YP_009513196.1.
SEQ ID NO: 31 is the amino acid sequence for the NDV phosphoprotein [Avian orthoavulavirus 1] from NCBI Reference Sequence: YP_009513195.1.
SEQ ID NO: 32 is the amino acid sequence corresponding to the NDV nucleocapsid protein [Avian orthoavulavirus 1] from NCBI Reference Sequence: YP_009513194.1.
SEQ ID NO: 33 contains the nucleic acid sequence for the C1 vector pMYT1140.
SEQ ID NO: 34 contains the nucleic acid sequence for the C1 vector pMYT1727.
SEQ ID NO: 35 contains the nucleic acid sequence for the C1 vector pMYT1729.
SEQ ID NO: 36 contains the nucleic acid sequence for the C1 vector pMYT1730.
SEQ ID NO: 37 contains the nucleic acid sequence for the C1 vector pMYT1731.
SEQ ID NO: 38 contains the amino acid sequence for a T4 fibritin timerization domain.
SEQ ID NO: 39 contains the amino acid sequence for a GCN4 trimerization domain.
SEQ ID NO: 40 contains a nucleic acid sequence for an IgY CH antibody fragment.
SEQ ID NO: 41 contains the amino acid sequence for an IgY CH antibody fragment.
SEQ ID NO: 42 contains a nucleic acid sequence for an IgY CL antibody. fragment.
SEQ ID NO: 43 contains the amino acid sequence for an IgY CL antibody fragment.
SEQ ID NO: 44 is an NDV nucleic acid sequence which encodes for a full-length hemagglutinin-neuraminidase (HN) protein (GenBank: MH377274.1).
SEQ ID NO: 45 is an NDV nucleic acid sequence encoding for a full-length NDV fusion (F) protein (GenBank: MH377274.1).

### FIELD

This disclosure concerns a method for producing viral antigens using genetic recombination and a fungal expression system, vaccines comprising the viral antigens, and embodiments of a method for using the vaccines.

### PARTIES TO JOINT RESEARCH AGREEMENT

Certain information disclosed herein resulted from a joint research collaboration between Phibro Animal Health Corporation and Dyadic International, Inc.

### BACKGROUND

Newcastle Disease (ND) is an economically burdensome disease which occurs in almost 230 species of bird. Outbreaks can be especially devastating on poultry farms. In chickens, the symptoms of the disease include respiratory distress, such as gasping, sneezing, and coughing; nervous symptom impairment as evidenced by drooping wings, twisted necks, and muscular tremors; and other signs of malaise, such as green watery diarrhea, and the production of thin-shelled and/or misshapen eggs. The mortality rate at eight days post exposure to the avian orthoavulavirus-1 causal agent, which is also referred to as avian Avulavirus, avian paramyxovirus-1, or Newcastle Disease virus (NDV), can be 90-100%. This virus is included in the Paramyxoviridae family, along with the causal agents of measles, mumps, and canine distemper. Different strains of the virus can cause different clinical signs of infection. Velogenic strains (highly virulent) can cause necro-hemorrhagic lesions in the intestine, resulting in up to 100% morbidity and mortality. Mesogenic strains (intermediate virulency) can cause respiratory and nervous system symptoms where infection can result in moderate morbidity and mortality. Meanwhile, lentogenic strains (low virulency) typically cause only minimal respiratory distress and are strains sometimes used for making attenuated vaccines.

Genomes of NDV strains typically range from 14.9 to 17.4 kilobases and are negative-sense, single-stranded RNA. There are at least six genes in the genomes, which include a nucleocapsid, a phosphoprotein, a matrix, a fusion protein, a hemagglutinin-neuraminidase attachment protein, and an RNA-dependent RNA polymerase. In part due to the lack of an error-checking function on RNA polymerases, these viruses readily mutate and can quickly differentiate into new strains, which often frustrates vaccination efforts. The fusion (F) and hemagglutinin-neuraminidase (HN) proteins are two glycoproteins essential to the virus's ability to establish a host cell infection.

The F protein is naturally present on the viral envelope of NDV. The protein is expressed as an inactive F₀ protein, which is then cleaved by host proteases into F₁ and F₂ subunits. These subunits then facilitate the fusogenic event, which leads to viral infection of the host cell. Specifically, the activated F protein subunits undergo a conformational change to create a pore in the host cell membrane through which the virus passes. The HN protein aids this process by initially recognizing and attaching to receptors on the host cell.

Virulence of a specific NDV strain depends on the amino acid sequence of the cleavage site of the F₀ protein. The oligobasic cleavage site motif (RRQR/KR) can be cleaved by more ubiquitous and promiscuous proteases and corresponds to the more virulent strains. Conversely, the monobasic cleavage site motif (GR/KQGR) can only be cleaved by less numerous, tissue-specific proteases, and corresponds to less virulent strains.

Despite the availability of commercial NDV vaccines, high mortality outbreaks still occur on poultry farms. The high mutation rate of the virus frequently leads to new strains and available vaccines do not always provide immunity for such new strains. Therefore, even vaccinated populations remain susceptible to a virulent, differentiated NDV strain. Thus, there is an urgent need for new, effective NDV vaccines, as well as a need for a method for quickly producing new antigens as NDV strains mutate.

### SUMMARY

Certain disclosed embodiments include a transgenic fungus cell comprising a recombinant gene that comprises a fungal promoter operably linked to a nucleic acid sequence encoding a Newcastle Disease virus antigenic peptide. One example of a suitable transgenic fungus is Thermothelomyces heterothallica (C1). Certain disclosed embodiments used Thermothelomyces heterothallica strains DNL155, DNL157, M5355, M5739, and strains producing different glycan patterns, such as Man₃₋₉, Man₃, G0, and/or G2.

The transgenic fungus can express recombinant antigenic peptides or fragments, such as from Newcastle Disease virus that elicit an immunogenic response from a subject, such as poultry. The antigenic peptides can be derived from any viral protein or portion thereof that elicits an immune response, such as NDV F proteins, NDV HN proteins, or a combination of NDV F proteins and NDV HN proteins. In some embodiments, the transgenic fungal cell contains a recombinant gene comprising a nucleic acid sequence encoding an antigenic peptide comprising a sequence of at least 90% similarity to sequences disclosed herein, such as SEQ ID NOs: 5, 8, 11, 14, 17, 20, 23, 26, 44, 45, or combinations thereof. Certain disclosed embodiments include using versions of the transgenic fungus cell that comprise a recombinant gene comprising a nucleic acid sequence corresponding to an amino acid sequence of at least 90% similarity to SEQ ID NOs: 3, 4, 6, 7, 9, 10, 12, 13, 15, 16, 18, 19, 21, 22, 24, 25, 27, 28, 30, 31, 32, or combinations thereof. In some embodiments, the episome pMYT1055 (SEQ ID NO: 29) can be used as a vector to transform the fungus with an antigenic peptide. In some embodiments, the recombinant antigenic peptide comprises a signal sequence, and in specific embodiments, this signal sequence corresponds to the CBH1ss secretion signal sequence (SEQ ID NO: 1). In some embodiments, the antigenic peptide sequence can be operably linked to a fungal promoter, for example, bgl8.

Other disclosed embodiments include a method for making a transgenic fungus cell by inserting foreign genes into the fungal cell's chromosome. This set of foreign genes can include a nucleic acid sequence encoding an antigenic peptide, such as an antigenic NDV protein or portion thereof, operably linked to a fungal promoter and a fungal terminator.

Recombinant antigenic peptides produced by a transgenic fungus also are disclosed. These recombinant antigenic peptides produce an immunogenic response in a subject, such as poultry. These antigenic peptides can comprise the amino acid sequences of SEQ ID NOs: 3, 4, 6, 7, 9, 10, 12, 13, 15, 16, 18, 19, 21, 22, 24, 25, 27, 28, 30, 31, or 32, or combinations thereof. A person of ordinary skill in the art will appreciate that the number of amino acids forming such peptides can vary, but typically such peptides comprise from 5 to 1000 amino acid residues, more typically 8 to 800 amino acid residues. In further embodiments, the amino acid residues of any of the antigenic peptides can be modified. In other embodiments, the antigenic peptide can be conformationally stabilized with the addition of a trimerization domain. The antigenic peptide can also be fused to an antibody or antibody fragment to selectively target the antigenic peptide to the immune cells of a subject.

One embodiment of a disclosed method concerns producing a recombinant antigenic peptide from a transgenic fungus by fermenting the fungus in a bioreactor and harvesting the recombinant antigenic peptides produced, either with or without purification. Purification can include filtering, C-tag affinity chromatography, histidine-tag affinity chromatography, size-exclusion chromatography, fast protein liquid chromatography, high performance liquid chromatography, affinity chromatography, hydrophobic interaction chromatography, ion exchange chromatography, reverse phase chromatography, immunoaffinity chromatography, ultracentrifugation, precipitation, and any and all combinations thereof. In one specific embodiment, raw fermentation materials are filtered to remove nontarget culture supernatant particles, and the target proteins are purified from the resulting filtrate by any suitable method, such as C-tag column binding and elution.

Harvesting recombinant antigenic peptides from the fermented transgenic fungus can also include extracting proteins from the fungal mycelium or biomass contents of the bioreactor. In another embodiment, the antigenic peptides are harvested from the supernatant of the bioreactor contents. In some embodiments, the bioreactor fermentation process is temperature controlled within a temperature range of from about 20° C. to about 40° C., and more particularly 25° C. to 38° C. In further embodiments, the bioreactor comprises a fed-batch technology fermenter.

Vaccine formulations comprising one or more antigenic proteins are also disclosed. In addition to an antigenic peptide produced by a transgenic fungus, disclosed vaccine formulations also typically include additional components, such as an oil, an emulsifier, and/or an adjuvant. Certain disclosed vaccine embodiments are oil-in-water emulsions, where the oil is any acceptable oil, such as mineral oil. The vaccine can comprise a single antigenic peptide, or may comprise two or more antigenic peptides, such as one or more peptides selected from SEQ ID NOs: 3, 4, 6, 7, 9, 10, 12, 13, 15, 16, 18, 19, 21, 22, 24, 25, 27, 28, 30, 31, or 32. The vaccine formulation can include combinations with other antigens, such as Bacterial antigens, inactivated viruses, recombinant proteins, and the like. The vaccine can comprise plural emulsifiers and/or adjuvants, such as sorbitol-T and sorbitan oleate. Disclosed vaccines can be administered as a combination with other vaccines or therapeutics.

Embodiments of a method of preparing a vaccine from a recombinant antigenic peptide produced by a transgenic fungus also are disclosed. The method comprises forming an oil-in-water emulsion comprising the antigenic peptide or peptides, and other suitable vaccine components, including oil, water, emulsifiers, and/or adjuvants.

A method of administering disclosed vaccine embodiments to a subject also are described. In one embodiment, the subject can be poultry, particularly chicken. In another embodiment, the subject is a human. The vaccine can be administered by any suitable method, such as spraying, dropping, painting, or injecting the subject with a vaccine formulation via nasal, oral, dermal, muscular, or optical contact. The vaccine may be in the form of a(n) aerosol, powder, liquid, or solid. The vaccine can also be administered by intramuscular or subcutaneous injection. A further embodiment comprises serial administration of two or more vaccine doses, such as a first administration to poultry followed by a second administration 14 to 35 days subsequent to the first dose.

### BRIEF DESCRIPTION OF THE DRAWINGS

The present application includes at least one drawing executed in color. Copies of the patent application publication or patent issuing from this application with color drawing(s) will be provided by the Office upon request and payment of the necessary fee.

FIG. 1 is a schematic diagram illustrating an exemplary process of this disclosure.

FIG. 2 is a scheme illustrating integration of two arms of an expression cassette designed using HygR split marker.

FIG. 3 shows the transmembrane domain prediction for SEQ ID NO: 3, the native NDV F protein.

FIG. 4 shows the transmembrane domain prediction for SEQ ID NO: 4, the native NDV HN protein.

FIG. 5 provides images of Western blot-based screening gels for recombinant F proteins expressed in DNL155-derived strains. Target protein detection was performed using chicken NDV antiserum and biotin-α-C-tag conjugate as primary detection agents and rabbit anti-chicken IgY-IRD680 (shown in red) and Streptavidin-IRD800 (shown in green), as secondary detection agents, respectively. Strains transformed with construct pMYT1358 express the full-length F protein with an expected product size of 55 kDa. Strains transformed with the construct pMYT1361 express a recombinant F protein with a truncated C-terminal transmembrane domain and a modified protease cleavage site. The expected product size of this construct is 50 kDa. The expected products were detected in both supernatant and mycelium lysates. The bands marked with an asterisk correspond to the transformed strain selected for fermentation and denoted as M5041.

FIG. 6 provides images of Western blot and stained protein gels that were used to assess optimization of fermentation conditions for expression of F proteins in the C1 expression systems, where: MT460 refers to the fermentation at +25° C. of DNL155 transformed with pMYT1358 carrying the full-length recombinant F protein expressed with an expected product size of 55 kDa; MT461 refers to the fermentation at +25° C. of DNL155 transformed with pMYT1361 carrying a truncated recombinant F protein with an expected product size of 50 kDa; and MT455 refers to the fermentation of the same construct in the same cell line but at +38° C. Target protein detection was performed with the primary detection agents NDV antiserum and biotin-α-C-tag conjugate and secondary detection agents rabbit anti-chicken IgY-IRD680 (shown in red) and Streptavidin-IRD800 (shown in green), respectively. Target protein was produced in all three strains. Lowering the growth temperature from +38° C. to +25° C. increased the production levels of the 50 kDa truncated recombinant F protein.

FIG. 7 provides images of gels used to assess the purification of NDV recombinant F proteins from supernatant produced in an MT455 bioreactor run. Here, 1.5 mL of the supernatant from run MT455-6 was collected after 7 days of fermentation and was purified on a CaptureSelect C-tag XL 1 mL column. The binding buffer comprised 1×PBS with 0.5 M NaCl and the elution buffer was 20 mM Tris-HCl, 2 M MgCl₂, with 1 mM EDTA pH 7.5. Target protein detection is shown with NDV antiserum/rabbit anti-chicken IgY-IRD680 in red and biotin-α-C-tag/Streptavidin-IRD800 in green. The majority of the target protein, with a predicted size of 50 kDa, elutes in the third and fourth fractions. No target protein was detected in the flow-through and wash fractions. The target protein co-elutes with the 70 kDa host protein Hsp70.

FIG. 8 (A) provides images of gels for screening C1 transformants expressing recombinant HN proteins. Transformants were screened in a 24-well plate culture format for the production of target recombinant protein. All constructs, except for pMYT1362, showed positive expression of the target protein in this experiment. The transformants indicated with asterisks were selected for further analysis. Target protein detection was performed using biotin-α-C-tag conjugate as a primary detection agent and Streptavidin-IRD800 (shown in green) as a secondary detection agent.

FIG. 8 (B) provides images of a gel showing screening of recombinant HN proteins expressed in C1 transformants. Transformants were screened in a 24-well plate culture format for the production of the target recombinant protein. The transformants indicated with asterisks were selected for further analysis. Detection was performed using biotin-α-C-tag/Streptavidin-IRD800 in green.

FIG. 9 provides images of gels used to assess the outputs of bioreactor fermentations of recombinant HN producing strains. MT441 was a fermentation run of DNL157 with pMYT1363 corresponding to expression of the full-length HN protein with an expected size of 63 kDa. MT442 was a fermentation run of a DNL155 isolate transformed with pMYT1364, which corresponds to the HN protein with a transmembrane domain truncation and an expected size of 55 kDa. Protein detection is shown with NDV antiserum/rabbit anti-chicken IgY-IRD680 in red and biotin-α-C-tag/Streptavidin-IRD800 in green. The target protein was produced in both settings. Äkta quantifications indicated higher protein production levels in MT441 with a Day 7 yield of 0.94 g/L, compared to an 0.56 g/L Day 7 yield using MT442.

FIG. 10 are images of gels used to analyze additional outputs from bioreactor fermentation runs comprising HN-producing strains. MT443 was a fermentation run conducted at +38° C. comprising a line of DNL155 transformed with pMYT1365, which corresponds to the truncated HN protein lacking the transmembrane domain with an expected size of 50 kDa. MT444 was a fermentation run of the same line but at +25° C. Protein detection is shown with NDV antiserum/rabbit anti-chicken IgY-IRD680 in red and biotin-α-C-tag/Streptavidin-IRD800 in green. The target protein was produced in both conditions.

FIG. 11 provides images of Western blot and stained protein gels analyzing purification of recombinant HN protein from the MT443 run. Here, 1.5 mL of the sample MT443-5 supernatant was purified on a CaptureSelect C-tag XL 1 mL column using a 1×PBS with 0.5 M NaCl binding buffer, and an elution buffer comprising 20 mM Tris-HCl, 2 M MgCl₂, and 1 mM EDTA, at a pH of 7.5. Target protein detection was performed using NDV antiserum and biotin-α-C-tag conjugate as the primary detection agents and rabbit anti-chicken IgY-IRD680 (shown in red) and Streptavidin-IRD800 (shown in green), respectively, as secondary detection agents. The majority of the target protein eluted in fractions 3, 4, and 5. No target protein was detected in the wash fractions.

FIG. 12 provides images of gels analyzing purified NDV recombinant F and HN proteins expressed using a C1 system on Western blot and stained protein gel under reducing and non-reducing conditions. Target protein detection was performed using NDV antiserum and biotin-α-C-tag conjugate as the primary detection agents and rabbit anti-chicken IgY-IRD680 (shown in red) and Streptavidin-IRD800 (shown in green), respectively, as secondary detection agents. Recombinant proteins HN-55 and HN-63 formed dimers under non-reducing conditions.

FIG. 13 is a bar graph comparing treatment groups of HI titers against genotype 7 (rLaSota-HN-G7) antigen after the first vaccine dose of 50 μg/dose in Example 2.

FIG. 14 is a bar graph comparing treatment groups of HI titers against genotype 7 (rLaSota-HN-G7) antigen after the second vaccine dose of 50 μg/dose in Example 2.

FIG. 15 is a bar graph comparing treatment groups of HI titers against genotype 2 (VH) antigen after the first vaccine dose of 50 μg/dose in Example 2.

FIG. 16 is a bar graph comparing treatment groups of HI titers against genotype 2 (VH) antigen after the second vaccine dose of 50 μg/dose in Example 2.

FIG. 17 shows the results of an ELISA-NDV-F with titers of the F50 treatment group after both doses in Example 2.

FIG. 18 shows the results of an ELISA-NDV-F with titers of the HN63+F50 treatment group after both doses according to Example 2.

FIG. 19 shows the HI titers against genotype 7 (rLaSota-HN-G7) antigen after one treatment dose according to Example 3.

FIG. 20 shows the HI titers against genotype 2 (VH) antigen after one treatment dose according to in Example 3.

FIG. 21 shows the NDV-F titers of F50 ELISA after one treatment dose according to Example 3.

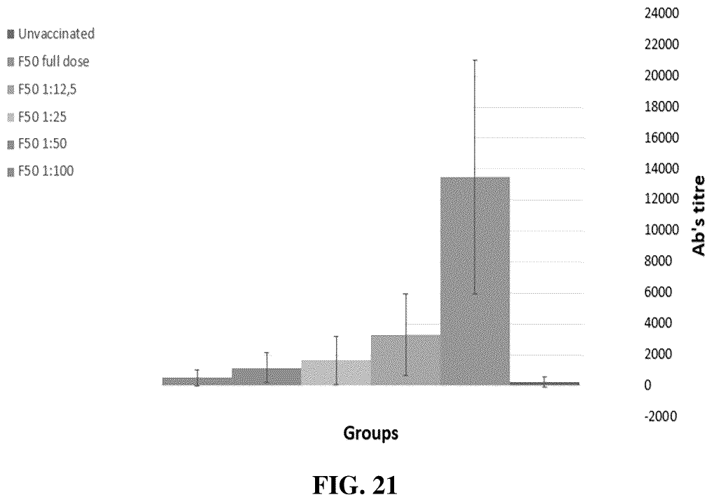

FIG. 22 is a comparison between NDV-F ELISA averages titers with STDEV of past study at a first bleeding time to Example 3 study at a second bleeding time.

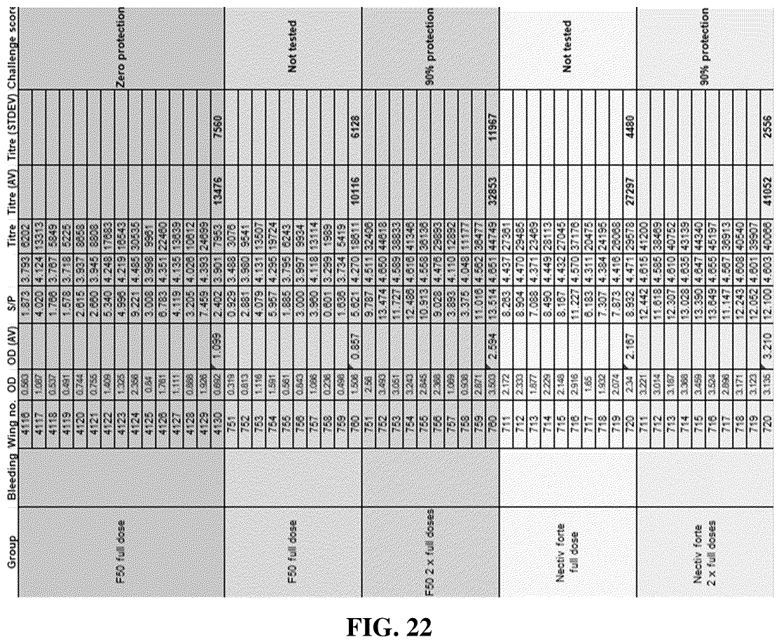

FIG. 23 shows viral secretions titers after the NDV challenge described in Example 3.

FIG. 24 provides images of gels used to analyze additional outputs from bioreactor fermentation runs comprising F-producing strains: MT642: (M6316 (M5824 (Δchi1)/2×NDV F+T4domain) at +25° C., and MT649:(M6318 (M5824 Δchi1)/2×NDV F+T4domain) at +32° C., where M6316 and M6318 are clones purified from two independent transformants. Target protein detection was performed using NDV antiserum/rabbit anti-chicken Ig-IRD680 (shown in red) and biotin-α-Ctag/Streptavidin-IRD800 (shown in green). NDV F production level improved achieved in the additional run to 1.7 g/L after six days, and 1.9 g/L after 7 days.

FIG. 25 provides images of gels analyzing purified C1 NDV F and HN antigens expressed using a C1 system on Western blot and stained gel under reducing and non-reducing conditions. Target protein detection was performed using NDV anti-serum/rabbit anti-chicken IgIRD 680 (shown in red) and biotin-α-Ctag/Streptavidin-IRD800 (shown in green); and a merge of the images (shown in yellow) where the signals overlap. Purified proteins exhibited good stability, and addition of a trimerization domain to NDV F antigen resulted in a molecular weight increase of approximately 5 kDa. Moreover, putative oligomers (dimers and trimers) can be seen in non-reduced samples of Phase III NDV F Antigen. NDV HN antigens produced in Phase I and Phase III show identical gel mobility. Production of NDV F antigen with trimerization domain preliminary estimated at about 1.9 g/L was achieved and NDV HN50 antigen with 4 copies of an HN50 expression cassette indicated that an estimated production level of 10 g/L was achieved.

FIG. 26 concerns size exclusion chromatography (UV) purification of NDV-F50 protein with trimerization domain with an expected monomer mass with oxidized disulfide bridges (10 cysteines) of 53376.3 Da (average mass by NIST protein calculator) analyzed by UV-DAD chromatography with PBS buffer, 12 mM NaH₂PO₄*H₂O, 150 mM NaCl pH 7.3 compared to a standard sample. The F50 sample showed broad early eluting peaks and two sharper peaks eluting at 7.85 and 8.14 min.

FIG. 27 is a graph and table (raw data) showing the standards exponential fit of sample calculated mass (kDa) versus retention time in minutes (RT, min) and the standards second order fit sample calculated mass (kDa) versus the retention time in minutes (RT, min). The calculated monomer mass was 53,376.3 Da, and the trimer therefore 106.1 kDa.

FIG. 28 provides a size exclusion chromatogram (UV-MS) of NDV-F50 protein with trimerization domain having an expected monomer mass with oxidized disulfide bridges (10 cysteines) of 53376.3 Da (average mass by NIST protein calculator) analyzed by UV-DAD chromatography with 40 mM ammonium acetate, unbuffered, and a pH of approximately 6 compared to untreated sample in PBS buffer, and to untreated sample in MS-buffer. The chromatograms in PBS and MS buffer are highly similar. PNGnaseF-treated sample differs significantly (removes N-glycans). PNGaseF treatment was done in minimally denaturing conditions (Rapid PngnaseF buffer 1:5 dilution, +Rapid PngaseF enzyme approximately 1:30 protein ratio, 4 hours at 40° C.)

FIG. 29 provides combined chromatograms of untreated sample versus the PngaseF-treated sample comprising NDV-F50 protein with trimerization domain at several time points. The spectrum at 8.5 minutes shows m/z dependent existence of monomeric and dimeric forms of 55 kDa protein and 110 kDa protein. The deconvolution of m/z region around 2,000 shows proteins around 50 kDa to 58 kDa and deconvolution of m/z region at 4,500 shows proteins around 106 kDa and 110 kDa, which likely represent dimers.

FIG. 30 provides combined and deconvoluted spectra from the main peak at 9.1 minutes and the dimeric and monomeric forms are shown by evidence of the trimeric protein. The monomeric deconvolution result at 53,381 Da is close to theoretical mass.

FIG. 31 shows the combined and deconvoluted spectra from main peak at 10 minutes (SEC predicted mass of 50 kDa). Deconvolution showing good quality masses at 49,445 Da and at 53,880 Da, the latter very close to theoretical mass. The +3.7 Da difference to theoretical could arise for example from open disulfide pair and deamidation.

FIG. 32 shows the results of the immunogenicity evaluation by testing the sera on NDV-F ELISA with Ab's titers in Example 5.

FIG. 33 shows the results of the immunogenicity evaluation by testing the sera on NDV-F ELISA with Ab's titers in Example 6.

### DETAILED DESCRIPTION

### I. Abbreviations

AAvV Avian Avulavirus
AH Animal house
C1 Thermothelomyces heterothallica
CBH1ss Cellobiohydrolase 1 secretion signal
CTL Cytotoxic T lymphocyte
EID50 50% embryo infectious dose
ELD50 50% embryo lethal dose
E-QCM English quality control methods
F Fusion protein
HI Hemagglutination inhibition assay
HN Hemagglutinin-neuraminidase
MHC Major histocompatibility complex
ND Newcastle Disease
NDV Newcastle Disease virus
PCR Polymerase chain reaction
PD50 Protective doses 50
PM Postmortem
QA Quality assurance
QCM Quality control method
IgY Immunoglobulin Y
SOP Standard operating procedure
SPF Specified pathogen free
vvNDV Velogenic vicerotropic Newcastle Disease virus
vNDV Velogenic Newcastle Disease virus
w/o Water in oil emulsion

### II. Terms and Definitions

Unless otherwise noted, technical terms are used according to conventional usage as would be understood by a person of ordinary skill in the art. Definitions of common terms in molecular biology may be found in Lewin's Genes X, ed. Krebs et al, Jones and Bartlett Publishers, 2009 (ISBN 0763766321); Kendrew et al. (eds.), The Encyclopedia of Molecular Biology, Blackwell Publishers, 1994 (ISBN 0632021829); Robert A. Meyers (ed.), Molecular Biology and Biotechnology: A Comprehensive Desk Reference, Wiley, John & Sons, Inc., 1995 (ISBN 0471186341); and George P. Rédei, Encyclopedic Dictionary of Genetics, Genomics, Proteomics and Informatics, 3rd Edition, Springer, 2008 (ISBN: 1402067534).

The following explanations of terms and abbreviations are provided to better describe the present disclosure and to guide those of ordinary skill in the art to practice the present disclosure. As used herein, “comprising” means “including” and the singular forms “a” or “an” or “the” refer to one or more than one unless the context clearly dictates otherwise. The term “or” refers to a single element of stated alternative elements or a combination of two or more elements, unless the context clearly indicates otherwise.

All publications, patent applications, patents, and other references mentioned herein are incorporated by reference in their entirety for all purposes. All sequences associated with the GenBank Accession Nos. mentioned herein are incorporated by reference in their entirety as of the present application's priority date. In case of conflict, the present specification, including explanations of terms, will control.

Although methods and materials similar or equivalent to those described herein can be used to practice or test the present disclosure, suitable methods and materials are described below. The materials, methods, and examples are illustrative only and not intended to be limiting. Other features of the disclosure will be apparent to a person of ordinary skill in the art from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

Amino acid residues in the disclosed sequence listing may be conservatively substituted or replaced by another residue with similar properties and characteristics. Typically, conservative substitutions have little to no impact on the activity of a resulting peptide. In one non-limiting example, a tyrosine residue in one peptide of a composition is substituted with a tryptophan residue. A peptide can be produced by chemical substitution to include one or more conservative amino acid substitutions, or can be produced by manipulating the nucleic acid sequence that encodes that peptide using, for example, standard procedures such as PCR or site-directed mutagenesis. Table 1 below provides conservative amino acid substitutions for expressly disclosed peptide sequences that are within the scope of the present disclosure.

TABLE 1
Conservative Amino Acid Substitutions

| DEFINITION | AMINO ACID | SYMBOL |
|---|---|---|
| Amino acids with aliphatic | Glycine | Gly—G |
| R-groups | Alanine | Ala—A |
| | Valine | Val—V |
| | Leucine | Leu—L |
| | Isoleucine | Ile—I |
| Amino acids with hydroxyl | Serine | Ser—S |
| R-groups | Threonine | Thr—T |
| Amino acids with sulfur- | Cysteine | Cys—C |
| containing R-groups | Methionine | Met—M |
| Acidic amino acids | Aspartic Acid | Asp—D |
| | Asparagine | Asn—N |
| | Glutamic Acid | Glu—E |
| | Glutamine | Gln—Q |
| Basic amino acids | Arginine | Arg—R |
| | Lysine | Lys—K |
| | Histidine | His—H |
| Amino acids with | Phenylalanine | Phe—F |
| aromatic rings | Tyrosine | Tyr—Y |
| | Tryptophan | Trp—W |
| Imino acids | Proline | Pro—P |

The following explanations of specific terms are provided to facilitate review of the various embodiments of this disclosure:

Adjuvant: The term “adjuvant” as used herein means any substance or vehicle that enhances the effectiveness of a disclosed immunogenic composition, such as by enhancing the immune response to an antigen (for example an NDV antigen) by an animal's immune system, such as a mammalian immune system. An adjuvant can be used to form a composition or compositions disclosed herein, for example as part of an ND vaccine composition. Adjuvants included in some embodiments of a composition disclosed herein can include, but are not limited to, sorbitan oleate, sorbitol-T, aluminum salts, such as aluminum phosphate or aluminum hydroxide; various types of oils, such as vegetable oil, mineral oil, oil-in-water based adjuvants, such as Emulsigen®, Emulsigen®-D, Emulsigen®-DL90, Emulsigen®-P, Emulsigen®-BCL, Emulsimune®, or TS6; Amphigen®; pluronic polyols; saponin-based adjuvants, such as saponin, Quil A, and QS-21; nonionic block copolymers; microfluidized emulsions, such as MF59; water-in-oil adjuvants, such as ISA 720, ISA 71 VG, ISA 35, ISA 51, or ISA 50V; water-in-oil-in-water based adjuvants, such as ISA 206 or ISA 201 (such as Montanide ISA 201 VG); Freund's complete adjuvant; Freund's incomplete adjuvant; polylactide glycolide (PLGA); toll-like receptor (TLR) ligand-based adjuvants, such as TLR7/8 adjuvants, such as R848 (Resiquimod); Carbomer-based adjuvants, such as those containing 934P or 971P; polymer-based adjuvants, such as CARBIGEN or POLYGEN: immune-stimulating complexes (ISCOMs); liposomes; polysaccharides; derivatized polysaccharides; oligonucleotides; cytokines; bacterial derivatives, such as trehalose-6,6-dibehenate (TDB) or cyclic diguanylate monophosphate (c-di-GMP); viral derivatives, such as polyinosinic-polycitidylic acid (poly (I:C)); or combinations thereof.

“Mucosally-adjuvanted” or “mucosal adjuvant” refer to an adjuvant or other compound, such as, for example, a polymer, that can interact with mucosal membranes and may stimulate an immune response. Additional information concerning mucosal adjuvants is provided by U.S. Pat. No. 10,279,031, which is incorporated by reference herein. Mucous membranes include the eye, oral, nasopharyngeal, anal, or vaginal membranes. The immune response that may be stimulated may include IgM, IgG, IgA, or a combination thereof. Compositions comprising such adjuvants may be applied to the mucosal membranes of an animal. Mucosal adjuvants may be “mucoadhesive,” in that they may adhere (generally non-covalently) to a mucosal membrane. Specific adjuvants with mucoadhesive properties include, but are not limited to, adjuvants comprising polymers, such as those comprising polyacrylic acids, such as carbomers and carbopols, or oil-in-water based adjuvants. Additionally, adjuvants containing nanoparticles may be used for intranasal administration. A person of ordinary skill in the art understands that a mucoadhesive adjuvant may contain one or a combination of any of the above adjuvants.

Administer, Administering, Administration: As used herein, administering a composition (e.g. an immunogenic composition) to an animal means to apply, give, or bring the composition into contact with the animal. Administration can be accomplished by a variety of routes, such as, for example, topical, oral, subcutaneous, transdermal, intrathecal, intramuscular, intravenous, intraperitoneal, intranasal, and similar routes, or combinations thereof.

As used herein, administering a composition mucosally includes directly administering the composition to an animal, such as by directly placing, such as, for example, spraying and/or dropping, the composition in the animal's mouth, nasal passages, or eye. Administering the composition mucosally also comprises providing the composition such that the animal administers the composition to itself, such as providing a composition for the animal to ingest. Exemplary methods of providing the composition include, but are not limited to, spraying the composition on the animal and/or otherwise topically applying the composition to the skin, or providing the composition in a form that the animal will eat. A person of ordinary skill in the art will understand that spraying may also facilitate direct administration because spray droplets may directly enter the mouth, nasal cavity, and/or eye of a swine. Another exemplary method of administering the composition to an animal is by intramuscular administration, such as, for example, by injection of a liquid formulation of the composition. Particular administration methods for poultry include in-ovo injection, intramuscular injection, subcutaneous injection, intradermal injection. Mucosal applications for poultry include eye drop, coarse spray, aerosol spray, gel spray, drinking water, and in feed application.

Disclosed compositions may be formulated for parenteral administration, such as, for example, by intradermal, intraarterial, intraperitoneal, intramuscular, subcutaneous, or intravenous routes, or combinations thereof. Examples of parenteral formulations of the compositions include, but are not limited to, suspensions that can be injected, solutions that can be injected, emulsions, and dry products that can be dissolved or suspended in an acceptable vehicle for injection. In addition, controlled-release parenteral formulations of the compositions can be prepared or administered, or both. Suitable materials for such administration include alcohols or a mixture or alcohols, such as a C₁-C₁₀ alcohol, such as ethanol, propanol, butanol, pentanol, hexanol, heptanol, octanol, nonanol, and/or decanol; polyols, such as polyethylene glycol; sterile water; glucose solution; saline solution; aqueous vehicles, such as, but not limited to, sodium chloride, dextrose, Dextrose Injection, Sodium Chloride Injection, Ringer's Injection, or Lactated Ringer's Injection, or combinations thereof; non-aqueous vehicles such as, but not limited to, ethyl oleate, peanut oil, corn oil, cottonseed oil, sesame oil, or isopropyl myristate, or combinations thereof; aqueous and non-aqueous isotonic sterile injection solutions, which can contain bacteriostats, buffers, antioxidants, or solutes that render the formulation isotonic within the blood of the recipient, or combinations thereof; and non-aqueous and aqueous suspensions that can be sterile and can include solubilizers, stabilizers, thickening agents, suspending agents, and preservatives, or combinations thereof. Formulations of the compositions can be presented in unit-dose or multi-dose containers, such as bottles, ampules, syringes, tubes, capsules, and vials.

Animal: “Animal” refers to a living multi-cellular vertebrate organism, a category that includes, for example, mammals and avian. The term mammal includes both human and non-human mammals. The term avian includes both wild and domestic species of birds, especially including chickens, geese, ducks, turkey, quail, Guinea fowl, peafowl, partridges, pigeons, emus, and pheasants.

Antibody: An “antibody” is an immunoglobulin molecule produced by B lymphoid cells. Antibodies are evoked in humans or other animals by a specific antigen (immunogen). Antibodies are characterized by reacting specifically with the antigen in some demonstrable way. “Eliciting an antibody response” refers to the ability of an antigen or other molecule to induce the production of antibodies.

Antigen: “Antigen” refers to a compound, composition, or substance that can stimulate the production of antibodies or a T-cell response in an animal, including compositions that are injected or absorbed into an animal.

Viral antigens suitable for use in the present technology include inactivated (or killed) virus and/or a viral peptide, peptides, protein, or proteins, that may be isolated, purified or derived from a virus. Viral antigens can be derived from viruses propagated on a substrate, such as a cell culture or other substrate, or they may be derived or expressed recombinantly, or they may be synthesized. Typically, viral antigens include, but are not limited to, epitopes which are exposed on the surface of the virus during at least one stage of a life cycle. Viral antigens may be conserved across multiple serotypes or isolates. Viral antigens include antigens derived from one or more of the viruses disclosed herein.

Attenuated, Attenuation: An “attenuated” virus is a virus that is weakened and/or less virulent as compared to a non-attenuated form of the virus, which may be capable of causing disease. Attenuated viruses may stimulate an immune response and/or immunity but are not capable of causing disease. Replication of an attenuated virus in culture and/or a recipient may be the same as, similar to, or different from that of a strain or strains from which the attenuated virus was derived. Attenuation may be achieved by altering a virus using one or more methods that involve a single step and/or multiple steps. For example, attenuating genetic modifications, such as, for example, attenuating mutations and/or genetic reassortment, may be introduced into coding and/or non-coding regions of a viral genome through site-directed mutagenesis, chemical methods, irradiation, and/or recombinant techniques. Such methods are well known to those of ordinary skill in the art. An attenuated form of an otherwise disease-causing virus may also be identified through culturing techniques, such as passaging, and/or may result from genetic differences in a viral genome not induced, created, or caused by human intervention. Methods of determining whether an attenuated virus maintains similar or reduced antigenicity as compared to the strain or strains from which the attenuated virus was derived are also well known to those of ordinary skill in the art. Such methods may include, for example, chemical selection and/or nucleic acid screening, such as, for example, by probe hybridization or PCR. Attenuated viruses, such as, for example, certain embodiments of viral vectors disclosed herein, may be used to stimulate an immune response and/or induce immunity in a recipient, such as an animal, such as a swine.

Combination: A combination includes two or more components that are administered such that the effective time period of at least one component overlaps with the effective time period of at least one other component. A component may be a composition. In some embodiments, the effective time periods of all components administered overlap with each other. In an exemplary embodiment of a combination comprising three components, the effective time period of the first component administered may overlap with the effective time periods of the second and third components, but the effective time period of the second component independently may or may not overlap with that of the third component. In an exemplary embodiment of a combination comprising four components, the effective time period of the first component administered overlaps with the effective time periods of the second, third, and fourth components; the effective time period of the second component overlaps with those of the first and fourth components, but not that of the third component; and the effective time period of the fourth component overlaps with that of the second and third components only. A combination may be a composition comprising the components, a composition comprising two or more individual components, or a composition comprising one or more components and another separate component (or components) or composition(s) comprising the remaining component(s). In some embodiments, the two or more components may comprise two or more different components administered substantially simultaneously or sequentially in any order, the same component administered at two or more different times, or a combination thereof.

Conditions sufficient for: The term “conditions sufficient for” refers to any environment that permits the desired activity, for example, that permits specific binding or hybridization between two nucleic acid molecules or that permits amplification and/or detection of a nucleic acid. Such an environment may include, but is not limited to, particular incubation conditions (such as time and/or temperature) or presence and/or concentration of particular factors, for example in a solution (such as buffer(s), salt(s), metal ion(s), detergent(s), nucleotide(s), enzyme(s), and so on).

Effective amount: The term “effective amount” or “therapeutically effective amount” or “immune-stimulatory amount” refers to the amount of an agent (such as one or more embodiments provided herein alone, in combination, or potentially in combination with other therapeutic agent(s)) that is sufficient to induce a desired biological result. That result may be amelioration or alleviation of the signs, symptoms, or causes of a disease, or any other desired alteration of a biological system. The amount can vary with the condition being treated, the stage of advancement of the condition, and the type and concentration of formulation applied. In some embodiments, an effective amount of an immune stimulatory composition is an amount which, when administered to a subject, is sufficient to engender a detectable immune response. Such a response may comprise, for instance, generation of an antibody specific to one or more of the epitopes provided in the immune stimulatory composition. Alternatively, the response may comprise a T-helper or CTL-based response to one or more of the epitopes provided in the immune stimulatory composition. All three of these responses may originate from naïve or memory cells. In other embodiments, a “protective effective amount” of an immune stimulatory composition is an amount which, when administered to a subject, is sufficient to confer protective immunity to the subject. Appropriate amounts in any given instance will be readily apparent to those of ordinary skill in the art or capable of determination by routine experimentation such as vaccination and observation of an antibody response or vaccination followed by a challenge wherein the vaccinated animal performs better than a non-vaccinated animal that is challenged similarly.

Encoding: “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (for example, rRNA, tRNA, and mRNA) or a defined sequence of amino acids and the biological properties resulting therefrom. Thus, a gene encodes a protein if transcription and translation of mRNA produced by that gene is capable of producing the protein, such as in a cell or other biological system. Both the coding strand, the nucleotide sequence of which is identical to the mRNA sequence and is usually provided in sequence listings, and noncoding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns, exons, or both.

Epitope: An “epitope” is an antigenic determinant. These are chemical groups or peptide sequences on a molecule that are antigenic, i.e. that elicit an immune response. T cell epitopes are presented on the surface of an antigen-presenting cell, where they are bound to MHC molecules. Professional antigen-presenting cells, such as macrophages, dendritic cells, and B cells, are specialized to present MHC class II peptides, whereas most nucleated somatic cells present MHC class I peptides. T cell epitopes presented by MHC class I molecules are typically peptides between 8 and 11 amino acids in length, whereas MHC class II molecules present longer peptides, 13-17 amino acids in length. An antibody specifically binds a particular antigenic epitope on a peptide, such as one or more immunogenic peptides selected from SEQ ID NOs. 3-28, and 30-32. In some examples a disclosed peptide is an epitope.

Expression: “Expression” refers to transcription and/or translation of a nucleic acid sequence. For example, a gene can be expressed when its DNA is transcribed into an RNA or RNA fragment, which in some examples is processed to form mRNA. A gene may also be expressed when its mRNA is translated into an amino acid sequence, such as a protein or a protein fragment. In a specific example, a heterologous gene is expressed when it is transcribed into an RNA. In another specific example, a heterologous gene is expressed when its RNA is translated into an amino acid sequence. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences: “Expression control sequences” are nucleic acid sequences that regulate the expression of a heterologous nucleic acid sequence to which they are operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

Expression vector: An “expression vector” is a vector comprising a recombinant polynucleotide comprising expression control sequences operatively linked to a nucleotide sequence to be expressed. An expression vector comprises sufficient elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

Glycoengineered: Using genetic engineering, the glycan patterns that a host cell adds to proteins it produces are artificially manipulated.

Host cell: “Host cell” refers to a cell or cells in which a vector can be propagated and its DNA expressed. The cell can be eukaryotic or prokaryotic. The cell can be mammalian, such as a swine cell. “Host cell” also includes any progeny of the subject host cell. It is understood that all progeny may or may not be identical to the parental cell since mutations may occur during replication. Such progeny are understood to be included when the term “host cell” is used.

Immune response: An “immune response” is a response of a cell of the immune system, such as a B-cell, T-cell, macrophage or polymorphonucleocyte, to a stimulus, such as an antigenic peptide. An immune response can include any cell of the body involved in a host defense response, including for example, an epithelial cell that secretes an interferon or a cytokine. An immune response includes, but is not limited to, an innate immune response or inflammation. As used herein, a protective immune response refers to an immune response that protects a subject from infection (prevents infection or prevents the development of disease associated with infection). Methods of measuring immune responses are known to those of ordinary skill in the art and include, for example, measuring proliferation and/or activity of lymphocytes (such as B or T cells), secretion of cytokines or chemokines, inflammation, antibody production and the like.

Immune stimulatory composition: The terms, “immune stimulatory composition” and “immunogenic composition” used herein refers to a composition useful for stimulating or eliciting an immune response (or immunogenic response) in a subject. The immune stimulatory composition can be a protein antigen, a nucleic acid molecule (such as vector) used to express a protein antigen, or a combination thereof. In some embodiments, the immunogenic response is protective or provides protective immunity, in that it enables the subject to better resist infection with or disease progression from the virus against which the immune stimulatory composition is directed.

Immunize: To render a subject (such as a bird, and particularly poultry) protected, through stimulation of the subject's immune system (such as by vaccination), from infection by an infectious disease (such as ND).

Immunogen: A compound, composition, or substance that can stimulate an immune response, such as the production of antibodies or a T-cell response in an animal, including compositions that are injected or absorbed into an animal. Particular non-limiting examples of immunogens include immunogenic peptides of SEQ ID NOs. 3, 4, 30, 31, and 32, constructs of SEQ ID NOs. 3, 4, 30, 31, and 32, domains of SEQ ID NOs: 3, 4, 30, 31, and 32, and/or full- and/or partial-length NDV proteins (for example, one or more proteins of SEQ ID NOs: 7, 10, 13, 16, 19, 22, 25, and 28), and/or nucleic acids, vectors, and/or host cells encoding such peptides, constructs, domains, and/or full- and/or partial-length NDV proteins.

Inactivated: In the context of the present disclosure, an “inactivated” virus is one that has been altered to the extent that it not capable of establishing an infection in a host or host cell. Viruses can be inactivated using, for example, chemicals, heat, alterations in pH and/or irradiation (such as ultraviolet or gamma irradiation). Inactivated viruses are also referred to as “killed.” A “chemically inactivated” virus is a virus that has been inactivated using a chemical method, such as treatment with betapropiolactone, formaldehyde, glutaraldehyde, 2,2′-dithiodipyridine or binary ethylene imine.

Infection: Infection or challenge means that the subject has been exposed to organisms that may produce disease causing the subject to suffer one or more clinical signs of the diseases when they have been exposed to such organisms.

Isolate, Isolated: An “isolated” biological component (such as a nucleic acid) has been substantially separated or purified away from biological or other components (for example biological components with which the component naturally occurs, such as chromosomal and extrachromosomal DNA, RNA, and proteins). Nucleic acids that have been “isolated” include nucleic acids purified by standard purification methods. The term also embraces nucleic acids prepared by recombinant expression in a host cell and subsequently purified, as well as chemically synthesized and purified nucleic acid molecules. Isolated does not require absolute purity, and can include, for example, nucleic acid molecules wherein at least 50%, 60%, 70%, 75%, 80%, 85%, 90%, 95%, 98%, 99%, or 99.9% of components in the original mixture with the desired materials are removed. As another example, an isolated biological component is one in which the biological component is more enriched than the biological component is in its natural environment within a cell, or other production vessel. An isolated nucleic acid may be in solution (e.g., water or an aqueous solution) or dried.

Newcastle Disease (ND): A disease that can occur in over 200 species of birds that causes respiratory, gastrointestinal, and nervous system symptoms. The disease can have a 100% mortality rate and is caused by the Newcastle Disease virus.

Newcastle Disease Virus (NDV): The causative agent of Newcastle Disease belonging to the family Paramyxoviruses (PMV). The genus of this virus is Avulavirus and includes nineteen avian PMV (APMV) serotypes. NDV is included under APMV serotype 1, a group which includes fifteen genotypes. This enveloped virus has a negative sense, single stranded RNA genome structure. Newcastle Disease virus has also been called avian paramyxovirus type 1, avian orthoavulavirus, and has been recently renamed avian Avulavirus.

Peptide: A “peptide” is a polymer having at least two amino acids joined by a peptide bond, and typically peptides comprise more than 2 amino acids joined together by amide bonds. Certain peptides, such as peptides having 25 or more amino acids, may be referred to as polypeptides. When the amino acids are alpha-amino acids, the L-optical isomer, the D-optical isomer, or combinations thereof, can be used. The term “peptide” as used herein is intended to encompass any amino acid sequence and includes modified sequences such as glycoproteins, and covers naturally occurring amino acid sequences, as well as those that are recombinantly or synthetically produced. The term “residue” or “amino acid residue” refers to an amino acid that is incorporated into a peptide. Exemplary peptides disclosed herein include the peptides of SEQ ID NOs. 7, 10, 13, 16, 19, 22, 25, and 28, constructs of SEQ ID NOs. 3, 4, 7, 10, 13, 16, 19, 22, 25, 28, and 30-32, domains of SEQ ID NOs: 3, 4, and 30-32, and NDV proteins, for example, of SEQ ID NOs. 3, 4, and 30-32.

Polynucleotide, Nucleic Acid Molecule: The term “nucleic acid molecule” or “polynucleotide” refers to a polymeric form of nucleotide of at least two bases in length, unless otherwise specified. A nucleic acid molecule may include both sense and anti-sense strands of cDNA, genomic DNA, RNA, and/or mixed polymers and/or synthetic forms of the above. The term “nucleic acid molecule” as used herein is synonymous with “nucleic acid” and “polynucleotide.” The terms include single- and double-stranded forms of DNA, unless specified otherwise. A polynucleotide may include either or both naturally occurring nucleotides and modified nucleotides linked together by naturally occurring and/or non-naturally occurring nucleotide linkages. The nucleotides can be ribonucleotides, deoxyribonucleotides, or modified forms of either nucleotide.

A recombinant polynucleotide includes a polynucleotide that is not immediately contiguous with both of the coding sequences with which it is immediately contiguous (one on the 5′ end and one on the 3′ end) in the naturally occurring genome of the organism from which it is derived. A recombinant nucleic acid molecule can also be one that is not naturally occurring or has a sequence that is made by an artificial combination of two otherwise separated segments of sequence. This artificial combination is accomplished by chemical synthesis or by artificial manipulation of isolated segments of nucleic acids, such as, for example, by genetic engineering techniques known to those of ordinary skill in the art. The term therefore includes, for example, a recombinant DNA molecule that is incorporated into a vector; into an autonomously replicating plasmid or virus; or into the genomic DNA of a prokaryote or eukaryote, or which exists as a separate molecule (for example, a cDNA) independent of other sequences.

Poultry: Any type of bird kept in a domestic or agricultural setting including, but not limited to, chickens, geese, ducks, turkey, quail, Guinea fowl, peafowl, partridges, pigeons, emus, pet birds, and pheasants.

Preventing: Preventing a disease refers to inhibiting the full development of a disease.

Treating: Refers to a therapeutic intervention that ameliorates a sign or symptom of a disease or pathological condition after it has begun to develop.

Ameliorating: Refers to a reduction in the number or severity of one or more signs or symptoms of a disease.

Promoter: A “promoter” is a minimal nucleic acid sequence sufficient to direct transcription. A promoter is typically located in the 5′ region adjacent to (and upstream of) the transcriptional start site of a gene, and generally contains a functional TATA box that directs the expression of the gene. A promoter generally contains both structural and functional elements and provides a control point for regulating the transcription of the associated gene. Also included are those promoter elements that are sufficient to render promoter-dependent gene expression controllable for cell-type specific, tissue-specific, or inducible by external signals or agents; such elements may be located in the 5′ or 3′ regions of the gene. Both constitutive and inducible promoters are included. For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Constitutive promoters may be used to drive expression of genes of interest in fungal cells, such as bgl8pr of Thermothelomyces heterothallica C1. Alternatively, synthetic constitutive promoters as described in the patent US20180371468A1 “Expression system for eukaryotic organisms” may be used to drive expression of the gene of interest in the eukaryotic cells. Examples of such promoters suitable for use in fungal cells include, but are not limited to, AnSESpr and/or TrSESpr. Promoters produced by recombinant DNA or synthetic techniques may also be used to provide for transcription of nucleic acid sequences.

A polynucleotide can be inserted into an expression vector that contains a promoter sequence, which facilitates the efficient transcription of the inserted genetic sequence of the host. The expression vector typically contains an origin of replication, a promoter, as well as specific nucleic acid sequences that allow phenotypic selection of transformed cells.

Purified: The term “purified” does not require absolute purity; rather, it is intended as a relative term. Thus, for example, a purified protein, virus, nucleic acid, or other compound is one that is isolated in whole or in part from associated proteins and other contaminants. In certain embodiments, the term “substantially purified” refers to a protein, virus, nucleic acid, or other compound that has been isolated from a cell, cell culture medium, or other crude preparation and subjected to purification to remove various components of the initial preparation, such as proteins, cellular debris, and other components.

Recombinant: A recombinant nucleic acid, protein, or virus is one that has a sequence that is not naturally occurring or has a sequence that is made by an artificial combination of two otherwise separated sequence segments. This artificial combination is often accomplished by chemical synthesis or, more commonly, by manipulating isolated segments of nucleic acids, for example, by genetic engineering techniques. The term recombinant includes nucleic acids, proteins and viruses that have been altered solely by addition, substitution, or deletion of a portion of a natural nucleic acid molecule, protein or virus.

Sample: A “sample” (or “biological sample”) refers to a specimen obtained from an organism, comprising, in certain embodiments, DNA (for example, genomic DNA or cDNA), RNA (including mRNA), protein, or combinations thereof. Examples include, but are not limited to isolated nucleic acids, cells, proteins, peptides, cell lysates, chromosomal preparations, tissues, and bodily fluids (such as blood, derivatives and fractions of blood (such as serum)), extracted galls, biopsied or surgically removed tissue (including tissues that are, for example, unfixed, frozen, fixed in formalin and/or embedded in paraffin), autopsy material, tears, milk, skin scrapes, surface washings, urine, sputum, cerebrospinal fluid, prostate fluid, pus, bone marrow aspirates, middle ear fluids, bronchoalveolar lavage, tracheal aspirates, nasopharyngeal swabs or aspirates, oropharyngeal swabs or aspirates, nasal washings, or saliva. In one example, a sample includes viral peptides, for example, specific to NDV. In particular examples, samples are used directly (e.g., fresh or frozen), or can be manipulated prior to use, for example, by extraction (for example of nucleic acids), fixation (e.g., using formalin) and/or embedding in wax (such as formalin-fixed paraffin-embedded tissue samples).

Sequence identity/similarity: The identity/similarity between two or more nucleic acid sequences, or between two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. In some embodiments, one or more disclosed peptides may comprise one or more amino acid sequences having at least 80% sequence identity (for example, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to an amino acid sequence or sequences of one or more peptides of SEQ ID NOs. 6, 7, 9, 10, 12, 13, 15, 16, 18, 19, 21, 22, 24, 25, 27, 28, 30, 31, or 32. In some embodiments, one or more disclosed nucleic acid molecules encoding one or more peptides of SEQ ID NOs. 5, 8, 11, 14, 17, 20, 23, 26, 44, or 45 may comprise one or more nucleic acid sequences having at least 80% sequence identity (for example, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%).

Sequence alignment methods for comparison and to determine sequence identity or similarity are known to those of ordinary skill in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, MD 20894) and on the internet, for use in connection with the sequence analysis programs blastp, blastn, blastx, tblastn and tblastx. Additional information can be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Subject: A “subject” is any multi-cellular vertebrate organism, a category that includes both human, non-human mammals (such as mice, rats, rabbits, sheep, swine, horses, cows, and non-human primates), and birds (such as chickens, geese, ducks, turkey, quail, Guinea fowl, peafowl, partridges, pigeons, emus, and pheasants). Certain disclosed embodiments of the present invention particularly concern birds, especially poultry.

Transformed: A “transformed” cell is a cell into which has been introduced a nucleic acid molecule using molecular biology techniques known to those of ordinary skill in the art. The term encompasses all techniques by which a nucleic acid molecule might be introduced into a cell, including transfection with plasmid vectors, transformation with viral vectors, and introduction of naked DNA by lipofection, electroporation, and/or particle gun acceleration.

Transformant: A “transformant” is a transformed clone, cell line, strain, or isolate resulting from a genetic transformation wherein recombinant foreign nucleic acid has been stably integrated into the host cell genome or is otherwise stably present in the host cell as genetic material capable of being expressed.

Vaccine: “Vaccine” refers to an immunogenic material, or a composition comprising an immunogenic material, capable of stimulating an immune response. Vaccines may be administered to prevent, ameliorate, or treat an infectious or other type of disease or diseases. The immunogenic material may include attenuated or inactivated microorganisms (such as bacteria or viruses), or antigenic proteins (including VLPs), peptides, or DNA derived from or encoding them, or combinations thereof. An attenuated vaccine is a virulent organism that has been modified to produce a less virulent form, but nevertheless retains the ability to elicit antibodies and an immune response against the virulent form. An inactivated vaccine is a previously virulent microorganism that has been killed with chemicals or heat, but which elicits antibodies against the virulent microorganism. Vaccines may elicit both prophylactic (preventative) and therapeutic responses. Methods of administration vary according to the vaccine, but may include inoculation, ingestion, inhalation, or other forms of administration. Vaccines may be administered with an adjuvant to boost the immune response.

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. An insertional vector is capable of inserting itself into a host nucleic acid. A vector can also include one or more selectable marker genes and other genetic elements. An expression vector is a vector that contains regulatory sequences that allow transcription and translation of an inserted gene or genes.

Virus-like particle (VLP): Virus-like particles are made up of one or more viral proteins but lack the viral genome. Because VLPs lack a viral genome, they are non-infectious.

### III. Overview of Embodiments

The production, composition, and use of immunogenic peptides, such as those from NDV, are disclosed herein. The production of these peptides involves the genetic transformation and growth of a fungal expression system. The composition of these peptides is supported by the disclosure of naturally occurring and/or synthetic nucleic acid and amino acid sequences in the form of plasmids, episomes, and/or chromosomally-integrated or genomically-integrated recombinant transgenes.

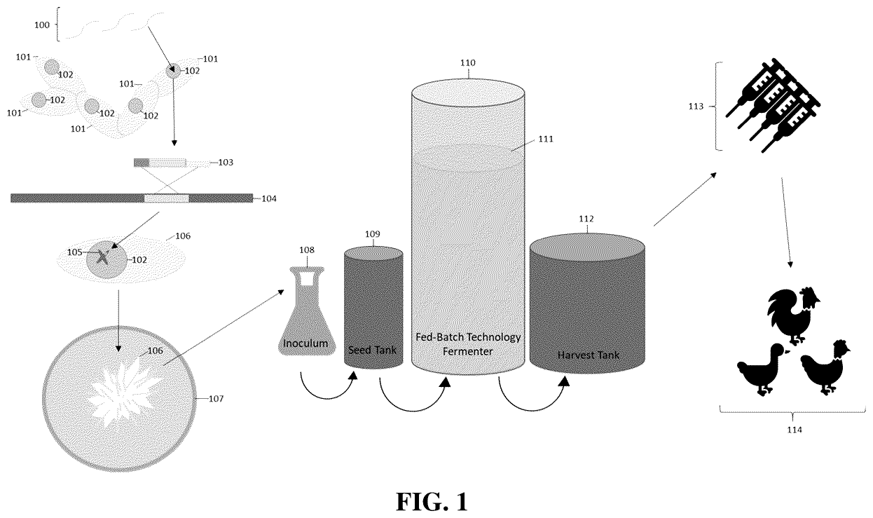

FIG. 1 provides an overview of a disclosed embodiment according to the present invention. Fragment 100 carries a target transgene 103 corresponding to nucleic acid sequence for a target antigenic protein. Fragment 100 is transferred into nuclei 102 of fungal expression system cells 101. Once the target gene 103 has entered the fungal nucleus 102, the target gene recombines with the fungal expression system genome 104 and stably integrates into the fungal expression system chromosome 105. Transformed fungal cells 106 can then be selected for, grown, and/or maintained in defined culturing conditions 107. Transformed fungal cells 106 can then be cultured as inoculum 108 for production of the target antigenic protein. After approximately 48 hours of culturing, inoculum 108 can then be used to inoculate the production seed tank 109. After 24 hours of fermentation in the seed tank, expression system cells 106 can then be transferred into the fed-batch technology tank 110 containing a defined medium 111. Here, specific fermentation conditions can be adjusted and controlled according to the specific needs of efficient production of the target protein from the expression system cells 106. After 120 to 168 hours of fermentation in the fed-batch technology tank 110, the contents are transferred to the harvest tank from which the target protein will be purified and/or enriched. Once the target protein has been harvested, it can be further processed into a vaccine composition 113. The vaccine composition 113 can then be administered to vaccinate and/or generate an immunogenic response in subjects 114.

A. Fungal Expression System

Thermothelomyces heterothallica (C1) is thermophilic ascomycete in the Sordariales order. It has been previously referred to as Myceliophthora thermophila and Sporotrichum thermophilum. The species was originally isolated in Russia from alkaline soils. The morphology of this fungus is unique and can be characterized as a fragmented, non-sporulating mycelial phenotype. In solid medium culture, the fungus grows as filamentous with white to light brown hyphae, depending on the strain.

The unique morphology of this fungus allows for low viscosity fermentation and standard sterilization can be used with bioreactors since this fungus is nonsporulating. Additionally, in some embodiments, culturing the fungus requires only a simple medium to produce high growth rates. C1 is also capable of high growth over a wide range of environmental conditions, including wide variations in pH and temperatures. These attributes make it possible to grow large quantities of the fungus at a low cost.

The C1 genome has been fully sequenced and annotated. The genome is roughly 38.74 Mb. C1 has been studied for its numerous enzymes capable of degrading plant cell wall components. Wildtype C1 naturally produces numerous proteases which can degrade heterologously expressed recombinant proteins. Therefore, a number of protease deletion strains have been created for the production of recombinant target proteins. One such strain is DNL155, which carries deletions of the following fourteen protease genes: alp1 alp2 pep4 prt1 srp1 alp3 pep1 mtp2 pep5 mtp4 pep6 alp4 alp7 kex2. Another disclosed strain is DNL157, which carries alp1 alp2 pep4 prt1 srp1 alp3 pep1 mtp2 pep5 mtp4 pep6 alp4 alp7 srp10 protease gene deletions. The deletion of protease genes helps prevent degradation of target recombinant proteins produced in these expression system strains. There are additionally chitinase-deletion strains (Δchi1) that may be used as expression system strains. These include, but are not limited to, strains M5355, M5739, and M5824.

An additional characteristic of the C1 fungal expression system is its potential for glycoengineering. The C1 glycan structure is more mammalian-like than yeast and other known microbial expression systems. Native C1 glycans are high in mannose-type glycans, such as Man3-Man9, and include low amounts of hybrid glycans. Successful glycoengineering of C1 allows for the creation of new cell lines with different glycan patterns, including strains that produce GlcNAc2Man₃ glycans, G0 glycans (e.g. 84% G0), galactosylated glycans (e.g. 26% G2, 45% G1 and 27% G0) and fucosylated glycans. In some embodiments, different glycoengineering in the C1 system can be used to modify or design target antigenic peptides.

The morphological, physiological, and genetic knowledge of C1 makes it a useful and effective expression system. The low-cost production and adaptability to varied culturing conditions allows for a rapidly scalable and adaptive bioreactor system capable of producing high yields of target proteins. Additional information concerning C1 is provided by WO/2019/038623, which is incorporated herein by reference.

B. Constructs

Embodiments of vectors capable of transforming the C1 genome with a target gene of interest include episomes and plasmids. One specific example is the vector backbone pMYT1055 (SEQ ID NO: 29), which contains a native C1 promoter, bgl8pr. Other expression vector backbones with bgl8pr include, but are not limited to, pMYT1730 (SEQ ID NO: 37) and pMYT1731 (SEQ ID NO: 38). Further examples are expression vectors with the synthetic promoter AnSESpr that include, but are not limited to, pMYT1727 (SEQ ID NO: 34) and pMYT1728 (SEQ ID NO: 35).

Antigenic peptides can also be engineered to include a trimerization domain used to stabilize certain conformations of the antigen. T4 fibritin provides one example that can be fused to the antigenic peptide as a trimerization domain to stabilize the pre-fusion configuration of the antigen. The T4 fibritin amino acid sequence is SEQ ID NO: 38. The GCN4 trimerization domain can also be used and includes, for example, SEQ ID NO: 39. Antigenic peptides can also optionally be fused to an antibody or an antibody fragment so that the antigen can be selectively targeted to certain immune cells. For example, an IgY antibody chain, such as CH or CL, can be conjugated to or genetically fused to an antigenic peptide. Examples of these antibodies or antibody fragments are provided by SEQ ID NOs: 40-43.

To produce a recombinant protein from the C1 expression system for downstream purification and harvesting, the protein can be engineered to be secreted from the expression host. For example, the Trichoderma reesei or native C1 cellobiohydrolase1 secretion signal sequence (CBH1ss) can be used. Alternatively, the signal peptides of Rhizopus oryzae amylase or Aspergillus awamori amylase, or any other compatible fungal origin or synthetic secretion signal, can be used. These signal peptides are cleaved leaving a mature protein.

Further, the target recombinant protein can be designed to include a purification tag. One embodiment uses a C-tag with the amino acid sequence EPEA (SEQ ID NO: 2) appended to the C-terminal end of the target peptide or protein sequence. Other examples of purification tags include polyhistidine, FLAG (DYKDDDK), albumin-binding protein tags, alkaline phosphatase, AU1 epitope (DTYRYI), AU5 epitope (TDFYLK), bacteriophage T7 epitope (T7-tag), biotin-carboxy carrier protein, bluetongue virus tag, calmodulin binding peptide, chloramphenicol acetyl transferase, cellulose binding domain, chitin binding domain, choline-binding domain, E2 epitope, galactose-binding protein, Glu-Glu, glutathione S-transferase, HaloTag®, histidine affinity tag, HSV epitope, KT3 epitope, LacZ, maltose-binding protein, Myc epitope, PDZ domain or ligand, polyarginine (Arg-tag), polyaspartate, polycysteine, polyphenylalanine, Profinity eXact®, S1-tag (NANNPDWDF), and streptavidin-binding peptide. Many of these purification tags can be appended to either the C-terminal or the N-terminal end of a recombinant protein.

Selection markers for genetic engineering with C1 can be genes conferring antibiotic resistance (e.g., against hygromycin or phleomycin), gene enabling utilization of acetamide or nitrate as a sole nitrogen source (Aspergillus nidulans amdS gene encoding acetamidase or T. heterothallica nia1 gene encoding nitrate reductase, respectively), or genes complementing auxotrophic mutations (e.g., C1 pyr4 gene). Specifically, aminoglycoside O-phosphotransferase-encoding gene HygR is a positive selection marker that allows for selection of hygromycin-resistant transformants on selective growth media supplemented with hygromycin B. Alternatively, Aspergillus nidulans amdS gene encoding acetamidase is a positive selection marker that allows for selection of transformants on minimal media supplemented with acetamide as a sole nitrogen source. Selection markers are an optional element and embodiments of this disclosure also include constructs that do not include a selection marker. Native, constitutive promoters can drive high expression of transgene in the fungus, although synthetic promoters have also been shown to be successful for transgene expression. For example, a native C1 promoter is bgl8 and can be paired with a downstream chi1 terminator.

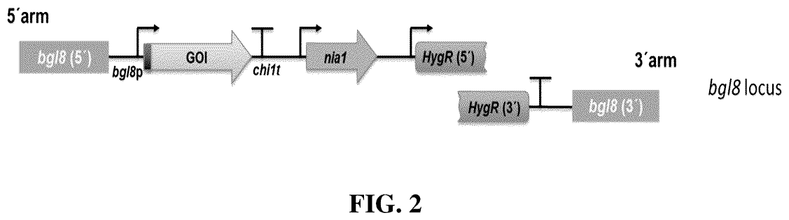

As seen in FIG. 2, the gene of interest (GOI) can be an antigenic region of an NDV protein. The bgl8 promoter is a native C1 promoter while HygR is a selection marker for hygromycin resistance. Only those transformants which have had the cassette stably integrated into genomic DNA will be resistant to hygromycin and express the target protein.

C1 can also be stably transformed to express recombinant antigenic proteins without a selection marker.

C. Transforming C1

The fungal expression system can be genetically transformed using a method which allows entry of foreign nucleic acid into the host cell's nucleus such that it can make contact with and stably integrate through recombination into the host cell's genome. Example 1 provides further detail on the genetic transformation process of C1.

The fungus can be genetically transformed using site directed integration and a selection marker. There are specific genomic integration sites which correlate to high expression of recombinant genes. However, random recombinant DNA integration approaches are also possible with this fungus for producing non-native proteins.

In some embodiments, site directed transformation generates genetically stable C1 cell lines in approximately two months. One or two or more genes can be integrated into the same site. The use of multiple genes in multiple integration sites can facilitate manufacturing.

The process can then be used for rapid response to new pathogens and diseases while still producing a high number of therapeutic doses. The process can be more flexible, scalable, and affordable than the use of other expression systems.

D. Fermentation Process

The fermentation process occurs in a bioreactor where the transformed fungus is grown to produce high levels of desired target peptides. Once the fungal expression host has been transformed with a target gene, a fungal transformant line can be grown to produce inoculum. Once sufficient inoculum is produced, in 48 hours for example, the inoculum can be transferred to the seed tank. In the seed tank, the transgenic fungus will grow, in some embodiments, for 24 hours. Then, the seed tank contents are used to initiate the fed-batch technology fermenter tank. The fermenter tank can contain defined or undefined media, which can be optimized to support high level production of the target protein. There can also optionally be glucose feeding, and NH₄OH inputs to control the fermentation pH. Once fermentation has been completed, for example after 120 to 168 hours, the contents can be moved into a harvest tank for target protein isolation. In some embodiments, production of a target protein can be achieved in 4-7 days using this batch fermentation process of a transgenic fungus.

This system allows controlling a wide range of environmental conditions. For example, the pH can be maintained at a specific value between pH 5 and pH 8, or a different pH value. The temperature can be maintained, for example, at a point between 20° C. and 45° C., or at a higher or lower temperature. Optionally, temperature can fluctuate to various temperatures over the fermentation process to optimize growing conditions for certain transgenic fungal strains and to optimize high target protein production.

The fermenter tank can range in size from 1 L to 500,000 L and can be single-use or stainless-steel bioreactor, for example. In some embodiments, protein production from a transgenic fungus does not need an inducer. In some embodiments, the target protein is secreted into the media during the fermentation step. At the end of a fermentation process, there can be, for example, 30-40% biomass of fungal cells produced and 60-70% supernatant produced.

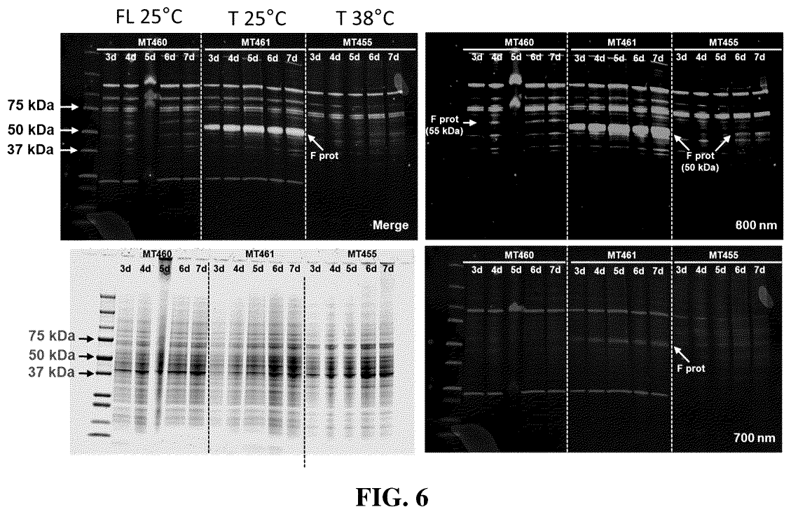

FIG. 6 provides an example of yield results for production of synthetic F proteins using different fermentation conditions with transgenic fungal cells.

E. Harvesting Target Recombinant Proteins

Target antigenic recombinant proteins can be optionally purified from fermentation batches prior to use in a vaccine. One example concerns filtering the fermentation batch supernatant. Another example includes harvesting fungal mycelium, extraction of protein therein, and purification or collection of the target antigen therefrom.

Target recombinant proteins can be purified using C-tag affinity chromatography, histidine-tag affinity chromatography size-exclusion chromatography, fast protein liquid chromatography, high performance liquid chromatography, affinity chromatography, hydrophobic interaction chromatography, ion exchange chromatography, reverse phase chromatography, immunoaffinity chromatography, ultracentrifugation, precipitation, other purification processes, or any and all combinations thereof.

The target recombinant protein can also be collected as a crude extract and used in a vaccine.

F. Viral Antigens

Disclosed herein are compositions that elicit an immunogenic response for vaccination against a viral pathogen. These compositions can, for example, be administered to animal subjects, particularly poultry. These compositions can be used to immunize a subject against ND, to ameliorate or eliminate the symptoms of ND, and/or to prevent a future high mortality outbreak of ND in an agricultural setting.

In some examples, the NDV F and HN proteins can be synthetically designed for expression in the C1 system and to elicit a strong immunogenic response from the subject. As the structure of the NDV F and HN proteins are strongly conserved between genotypes of APMV1, similar modifications and amino acid changes between genotype homologs can yield similar expression results in C1 and immunogenic responses in subjects. Therefore, this expression system platform is highly amenable to the functional homologs of the NDV F and HN proteins for all fifteen NDV genotypes.

G. Methods of Producing Vaccines

Embodiments of generating a vaccine include an optional purification of a target antigen. The antigen can be collected from the batch supernatant or from extraction of mycelial tissue or from any other cells or material in contact with the expression system producing the target antigen. Optionally, the target antigen can be collected without purification prior to vaccine preparation. The supernatant can be filtered prior to collection or purification.

Once collected, the antigen can be prepared, for example, as an oil-in-water emulsion comprising a water phase and an adjuvant or adjuvants. One, two, or more types of emulsifiers can be used, or a combination thereof, in the vaccine preparation. Any suitable oil can be used to prepare the emulsion, such as mineral oil, optionally DRAKEOL white mineral oil. The emulsifier(s) can be lipophilic surfactants, such as sorbitan esters, lipophilic solubilizers, monoesters, triesters, and triesters of lauric, stearate or oleic fatty acids, or a combination thereof. Lipophilic surfactants can also include polysorbates, hydrophilic solubilizers, or polyethoxylated sorbitan esters, or any combination thereof. One specific example of a sorbitan ester-based lipophilic surfactant is MONTANE. An example of a polyethoxylated sorbitan esters lipophilic surfactant is MONTANOX.

H. Methods of Administering Vaccine

Embodiments of peptides, immunogenic compositions, vectors, cells, and/or nucleic acid constructs can be administered to a human or an animal by any of the routes typically used for introducing a pharmaceutical composition or compositions into an animal. Methods of administration include, but are not limited to, intramuscular, oral, intravenous, intradermal, intraperitoneal, subcutaneous, parenteral, mucosal, rectal, vaginal, inhalation, intranasal, or combinations thereof. Parenteral administration, such as, for example, intramuscular, intravenous, or subcutaneous administration, is commonly achieved by injection. Administration can be local or systemic, or combinations thereof. Injectables can be prepared, for example, as emulsions, as solid forms suitable for solution or suspension in liquid prior to injection, or as liquid suspensions or solutions. Injection suspensions or solutions can be prepared from sterile powders, tablets, granules, or similar, or combinations thereof.

The composition or compositions administered to a human or animal may be administered with at least one acceptable carrier. Acceptable carriers are determined in part by the particular composition being administered, as well as by the particular method used to administer the composition. Thus, there is a wide variety of acceptable formulations of compositions of the present disclosure.

Preparations for parenteral administration include sterile aqueous or non-aqueous solutions, suspensions, and/or emulsions, such as, for example, oil-in-water and/or water-in-oil emulsions. Preparations for parenteral administration can also include adjuvants and/or polymers, such as, for example, CpG oligodeoxynucleotides (CpG ODN), Carbigen, Polygen, ISA 201 or 206 (such as Montanide ISA 201 VG), Quil-A, trehalose-6,6-dibehenate (TBD), toll-like receptor (TLR) ligand-based adjuvants (such as TLR7/8 adjuvants, such as R848 (Resiquimod)), cyclic diguanylate monophosphate (c-di-GMP), polyinosinic-polycytidylic acid (poly (I:C)), or combinations thereof. Examples of non-aqueous solvents are alcohols or glycols, such as propylene glycol, polyethylene glycol, vegetable oils such as olive oil, and injectable organic esters such as ethyl oleate. Aqueous carriers include water, alcoholic/aqueous solutions, emulsions, or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and similar. Preservatives and other additives may also be present, such as, for example, antimicrobials, antioxidants, chelating agents, inert gases and similar.

In some examples, the vaccine is administered via intramuscular injection into pectoralis tissue of poultry, or by intranasal administration, where the disclosed compositions can include one or more biodegradable, polymeric carriers that interact with one or more mucosal membranes. Polymers such as polylactide-co-glycolide (PLGA), chitosan (for example in the form of chitosan nanoparticles, such as N-trimethyl chitosan (TMC)-based nanoparticles), alginate (such as sodium alginate), carbopol, and carbopol-based polymers can be included. The composition can include one or more hydrophilic polymers, such as sodium alginate or carbopol, for example in combination with starch. The composition can be formulated as a particulate delivery system. Thus, the composition can include liposomes, immune-stimulating complexes (ISCOMs) and/or polymeric particles, such as virosomes. The compositions can also include one or more lipopeptides of bacterial origin, or their synthetic derivatives, such as Pam3Cys, (Pam2Cys, single/multiple-chain palmitic acids and lipoamino acids (LAAs). The compositions can also include one or more adjuvants, such as, for example, one or more of CpG oligodeoxynucleotides (CpG ODN), Flt3 ligand, Carbigen, c-di-GMP, poly (I:C), and monophosphoryl lipid A (MLA).

I. Timing of Administration

Disclosed compositions may be administered as a single dose or as multiple doses (for example, boosters). In some examples, a first administration is followed by a second administration. For example, the second administration can be with the same, or with a different composition than the first composition administered. In one specific non-limiting example, the second administration is with the same composition as the first composition administered. In another specific non-liming example, the second administration is with a different composition than the first composition administered. In one specific example, the first dose is administered three weeks (e.g., 19 to 22 days) prior to the second dose.

J. Dosage

The dose administered to a subject should be sufficient to induce a beneficial therapeutic response in a subject over time, or to inhibit viral infection. The dose can vary from subject-to-subject depending on the species, age, weight, and general condition of the subject, the severity of the infection being treated, whether the dose is being used to treat, alleviate, or inoculate against an infection, the particular composition being used, and/or the mode of administration. An appropriate dose can be determined by one of ordinary skill in the art using routine experimentation. And different antigens can be combined in different concentrations in doses. However, for certain disclosed embodiments, a suitable dosage range is 5 to 100 μg protein per dose, more typically 5 to 50 μg protein per dose, for the HN protein. And for the F protein, a suitable dosage range is 5 to 150 μg protein per dose, more typically 5 to 100 μg protein per dose. Examples 2 and 3 provide further details on dosage ranges.

A person of ordinary skill in the art will also appreciate that more than one dose may be beneficial or required. For example, it currently is believed that two administrations may be beneficial for HN or F protein immunogenic compositions.

### EXAMPLES

The following examples are provided to illustrate certain features and/or embodiments of the disclosure. These examples should not be construed to limit the disclosure to the particular features or embodiments described. Changes therein and other uses that are encompassed within the spirit of the invention as defined by the scope of the claims will occur to those of ordinary skill in the art.

### Example 1

### Producing F and HN Protein Antigens for Newcastle Disease in C1 System

### Background

Constructs were designed to provide optimal expression and yield using the C1 fungal expression system. Protein sequences for the F (SEQ ID NO: 3) and HN (SEQ ID NO: 4) proteins of target NDV Genotype VII were collected and analyzed. The vector backbones pMYT1055, pMYT1727, pMYT1728, pMYT1730, and pMYT1731 were used for adding a target cassette for transformation in the C1 expression system. The pMYT1055 vector carries the native C1 bgl8 promoter and hygromycin resistance selection marker. The vectors pMYT1727 and pMYT1728 carry the synthetic AnSES promoter and split amdS selection marker. The vectors pMYT1730 and pMYT1731 carry the native C1 bgl8 promoter and split amdS selection marker. Recombinant sequences were designed to be compatible with the corresponding vector backbones, as follows, to reach production and secretion of target NDV antigens from the C1 system. A summary of the constructs used is presented by Table 2.

### F Protein Constructs

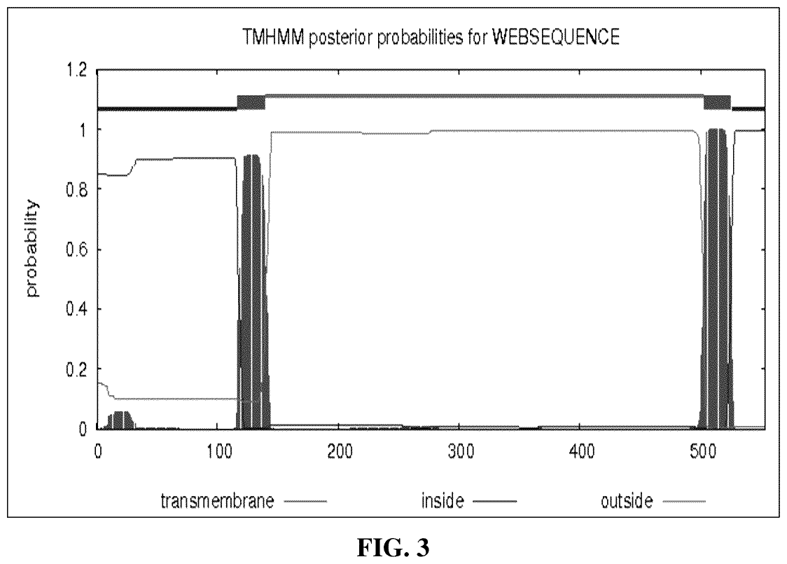

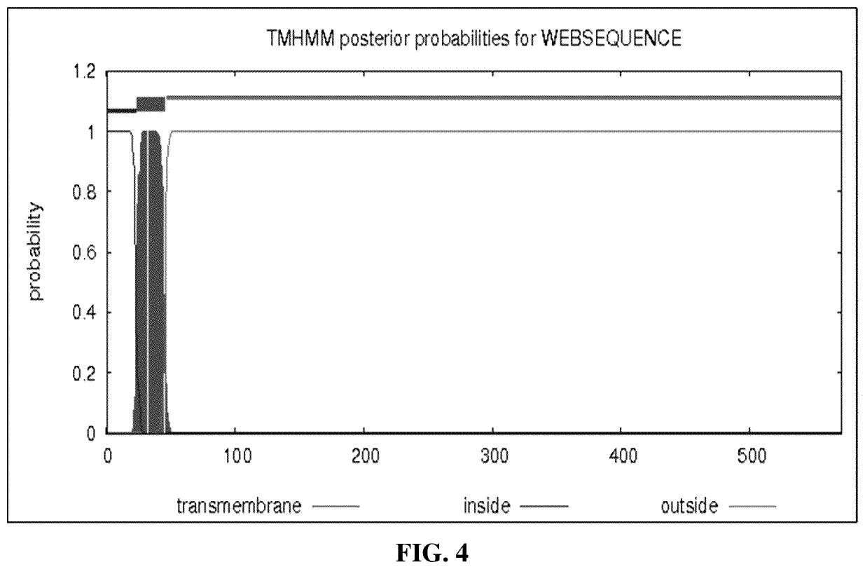

NDV F protein constructs were designed considering the unique characteristics of the native protein. For example, there are two transmembrane domains present in the native F protein structure which can be removed from the construct to increase expression and solubility of the target antigenic protein (FIG. 3). There is one transmembrane domain in the native HN protein as seen in FIG. 4 which can also be removed to improve solubility of the recombinant protein.

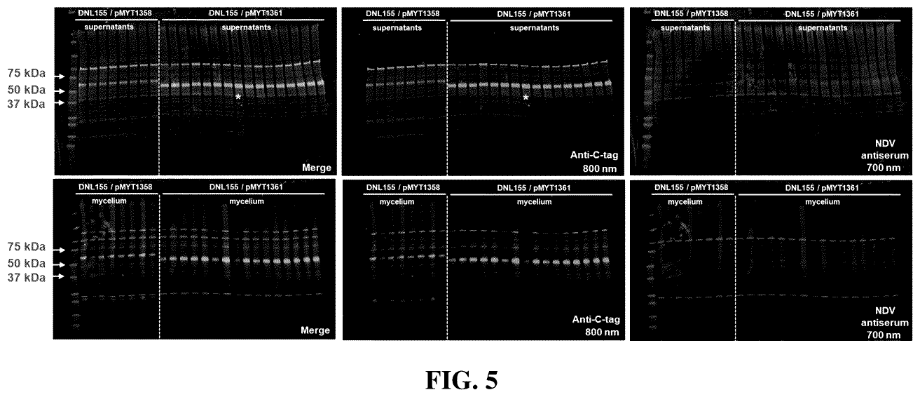

Construct pMYT1358 contains a cassette with the CBH1 secretion signal and residues 32-553 of the full length NDV F protein (SEQ ID NO: 5). Residues 1-31 of the full-length protein correspond to the native NDV signal peptide and are removed to allow for the appendage of the C1 fungal secretion signal CBH1ss. Further, a C-tag with an amino acid sequence of EPEA is added to the C-terminus of the target protein for purification purposes. The mature target protein (SEQ ID NO: 7) of this construct has an expected size of 55 kDa. Expression of the target protein from DNL155 transformed cells corresponding to strain M5038 is shown in FIGS. 5-6.

The pMYT1359 construct carries the information to provide for the expression of a target 50 kDa F protein fragment (SEQ ID NO: 10). Here, the C-terminal transmembrane domain of the protein has been removed so that only residues 32-499 of the native F protein are included in the expressed recombinant protein (SEQ ID NO: 8 and 9). There is also a C-terminal C-tag for protein purification methods as well as the native C1 CBH1ss appended to the N-terminus of the protein (SEQ ID NO: 8 and 9).

In another example, the pMYT1360 construct carries an F protein fragment with both of the transmembrane domains removed (SEQ ID NO: 11 and 12). Here, only residues 146-499 of the native F protein are used in the recombinant protein. The mature recombinant protein has an expected size of 38.5 kDa and is also amended with a C-terminal C-tag for purification and a CBH1ss (SEQ ID NO: 13).

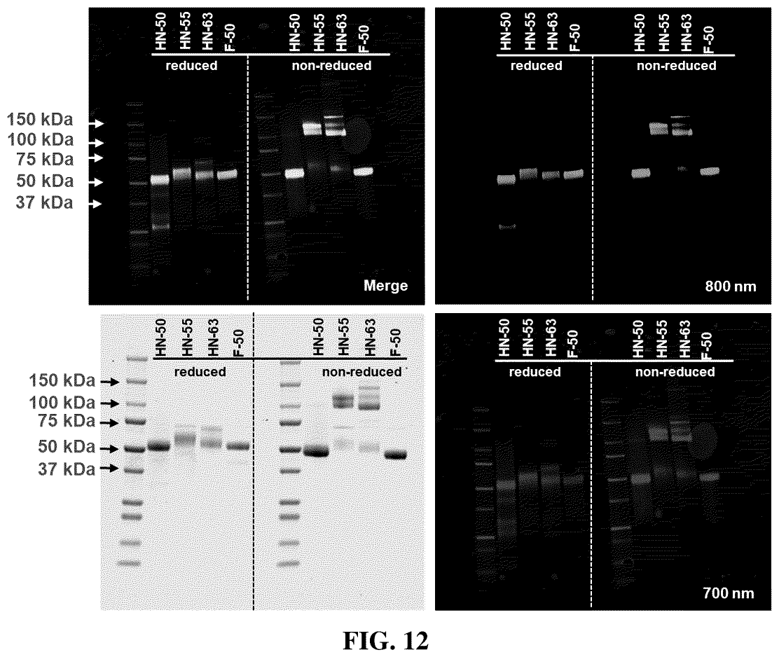

The constructs pMYT1361, pMYT1736, pMYT1737, and pMYT1738 contain a recombinant protein sequence carrying a mutation in the cleavage site motif (SEQ ID NO: 14, 15, and 16). Specifically, there is point mutation replacing lysine at position 115 with glycine (K115G). This mutation alters the protease cleavage site in F so that it is less likely to be cleaved and can therefore be more stable in vivo when administered as an antigenic protein to elicit an immunogenic response. The recombinant protein has had the C-terminal transmembrane domain removed and also has an N-terminal CBH1ss and a C-terminal C-tag amendment. The mature recombinant protein has an expected size of 50 kDa (SEQ ID NO: 16). Confirmed purification of this recombinant protein from strain M5041, a pMYT1361 transformed DNL155 strain, is shown in FIGS. 5, 6, 7, and 12.

### HN Protein Constructs

The native HN NDV protein is an outer viral membrane localized glycoprotein. Residues 24-46 of the amino acid sequence for the protein correspond an N-terminal transmembrane domain (FIG. 4). Truncating this N-terminal transmembrane domain can allow for increased recombinant protein expression without affecting the antigenic region of the protein. There is no predicted signal peptide in the sequence and the lack of a signal peptide for the protein has been confirmed by experimental data. However, a signal peptide can be amended for recombinant expression purposes.

The pMYT1362 construct carries the full length HN protein under the bgl8 promoter and with a C-tag of EPEA appended to the C-terminus (SEQ ID NOs: 17 and 18). No signal peptide was added to the N-terminus. The expected size of the expressed protein from this construct is 63 kDa (SEQ ID NO: 19).

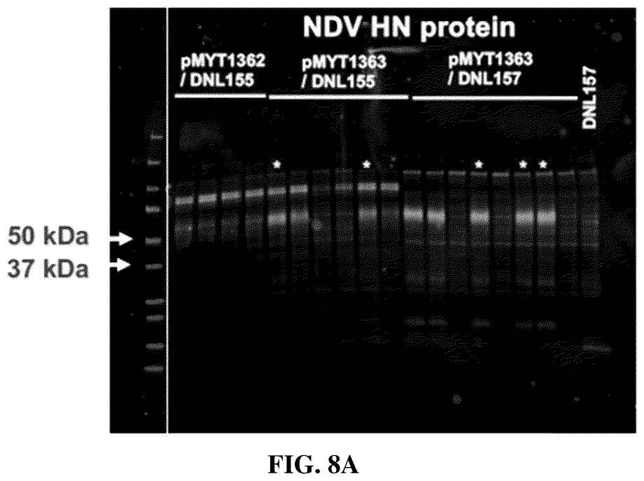

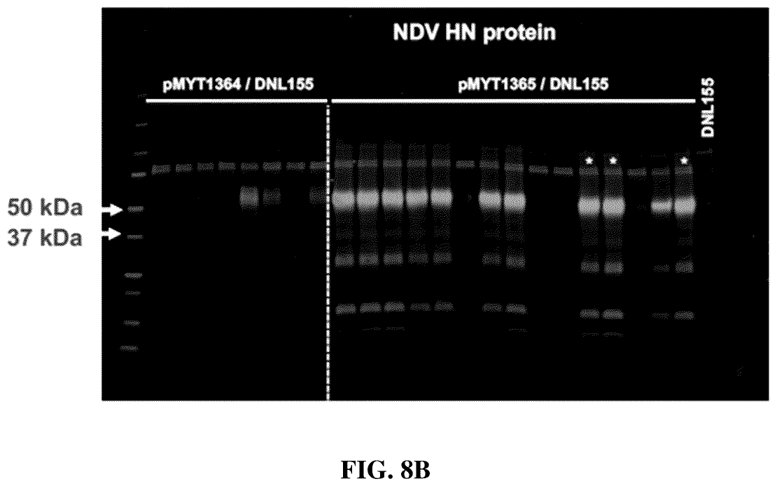

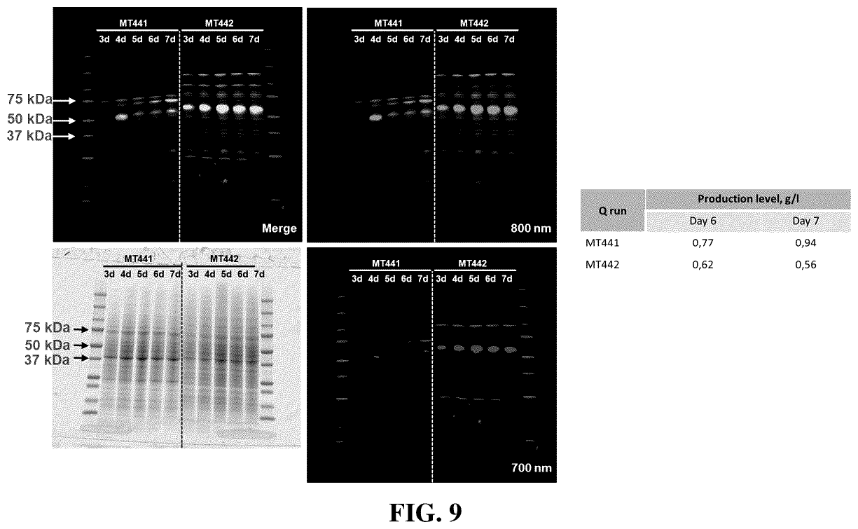

While pMYT1363 yields the same expected size of the mature protein at 63 kDa (SEQ ID NO: 22), the recombinant protein in this construct has the CBH1ss added to the N-terminus (SEQ ID NO: 21). This recombinant protein is also under the control of a bgl8 promoter. The confirmed purification of this protein from this construct in C1 is shown in FIGS. 8, 9 and 12.

The pMYT1364 construct carries the HN protein with the N-terminal transmembrane domain truncated such that the recombinant protein begins at residue 78 of the full-length native protein (SEQ ID NOS: 23 and 24). A C-tag is appended to the C-terminal end and CBH1ss is added to the N-terminus. The expected mature size of this protein is 55 kDa (SEQ ID NO: 25) and confirmed purification from fermentation of C1 transformed with this construct is shown in FIGS. 8, 9 and 12.

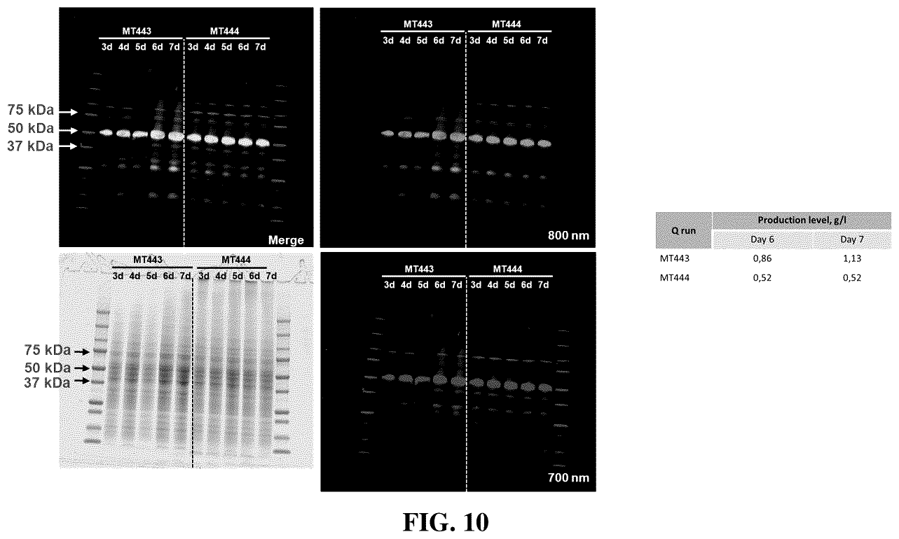

The constructs pMYT1365, pMYT1741, pMYT1742, and pMYT1743 hold a further truncated HN protein which begins at residue 124 of the full-length protein (SEQ ID NOS: 26 and 27). These constructs also include the C-tag for purification and the CBH1ss. Confirmed purification of this recombinant protein is shown in FIGS. 8, 10, 11, and 12 with its expected size of 50 kDa (SEQ ID NO: 28).

### Methods

Recombinant NDV F and HN antigens derived from NDV Genotype VII were expressed in protease-deficient C1 strains DNL155 and DNL157. Seven expression constructs were designed for each antigen. The coding sequences for both full-length and truncated forms (lacking transmembrane domains) of F and HN antigens with C-terminal C-tag EPEA appended were codon-optimized for their efficient expression in C1 system, placed in the context of C1 CBH1 signal sequence and embedded into insertion vectors that contained appropriate 5′ and 3′ regulatory sequences to drive a constitutive strong expression of the gene of interest, appropriate selectable marker gene cassette and targeting arms for the integration of the construct into well-defined hot spots of the haploid Thermothelomyces heterothallica C1 genome.

Synthetic fragments were designed containing sequences for full-length versions of NDV F and HN antigens with CBH1ss and C-tag appended to their 5′ and 3′ termini, respectively, and flanked by recombination sequences to appropriate C1 expression vectors and MssI restriction enzyme recognition sites. The fragments were synthetized by GENSCRIPT. The codon usage of the protein-coding sequences was optimized for their efficient expression in Thermothelomyces heterothallicus C1 expression system.

In one instance, the synthetized fragments were released from the GENSCRIPT constructs by digestion with the restriction enzyme MssI and cloned by Gibson Assembly (NEBUILDER HiFi DNA Assembly Cloning Kit, New England Biolabs) method into the PacI site of the C1 expression vector pMYT1055 under endogenous C1 bgl8 promoter and C1 chi1 terminator. The correct sequence of the constructs was confirmed by sequencing the fragments inserted into the plasmid. Plasmids of correct sequence carrying expression cassette for full-length NDV F (SEQ ID NO: 5) and NDV HN (SEQ ID NO: 20) proteins were given the plasmid numbers pMYT1358 and pMYT1363, respectively.

In a second instance, fragments coding for truncated versions of NDV F and HN antigens were amplified by PCR from the GenScript constructs using oligonucleotide primers with appended recombination sequences to appropriate C1 expression vector. Obtained PCR fragments were cloned by Gibson Assembly method into the PacI site of the C1 expression vector pMYT1055 under endogenous C1 bgl8 promoter and C1 chi1 terminator. The correct sequence of the constructs was confirmed by sequencing the fragments inserted into the vector. Plasmids of correct sequence carrying expression cassette for truncated versions of NDV F antigen were given the plasmid numbers pMYT1359 (SEQ ID NO: 8), pMYT1360 (SEQ ID NO: 11), and pMYT1361 (SEQ ID NO: 14), respectively. Plasmids of correct sequence carrying expression cassette for different versions of NDV HN antigen were given the plasmid numbers pMYT1362 (SEQ ID NO: 17), pMYT1364 (SEQ ID NO: 23), and pMYT1365 (SEQ ID NO: 26), respectively.

In a third instance, fragments coding for truncated versions of NDV F (SEQ ID NO: 14) and HN (SEQ ID NO: 26) antigens were amplified by PCR from the GENSCRIPT constructs using oligonucleotide primers with appended recombination sequences to appropriate C1 expression vectors. Obtained PCR fragments were cloned by Gibson Assembly method into the PacI site of the C1 expression vector pMYT1730 under control of endogenous C1 bgl8 promoter and C1 chi1 terminator. The correct sequence of the constructs was confirmed by sequencing the fragments inserted into the vector. Plasmids of correct sequence carrying expression cassette for truncated versions of NDV F and NDV HN antigens were given the plasmid numbers pMYT1736 and pMYT1741, respectively. PCR fragments with appropriate recombination sequences were also cloned into the PacI site of the C1 expression vector pMYT1731 under control of endogenous C1 bgl8 promoter and C1 bgl8 terminator. The correct sequence of the constructs was confirmed by sequencing the fragments inserted into the vector. Plasmids of correct sequence carrying expression cassette for truncated versions of NDV F and NDV HN antigens were given the plasmid numbers pMYT1738 and pMYT1743, respectively.

In a fourth instance, fragments coding for truncated versions of NDV F (SEQ ID NO: 14) and HN (SEQ ID NO: 26) antigens were amplified by PCR from the GENSCRIPT constructs using oligonucleotide primers with appended recombination sequences to appropriate C1 expression vectors. Obtained PCR fragments were cloned by Gibson Assembly method into the PacI site of the C1 expression vector pMYT1727 under control of the strong synthetic AnSES promoter and C1 chi1 terminator. The correct sequence of the constructs was confirmed by sequencing the fragments inserted into the vector. Plasmids of correct sequence carrying expression cassette for truncated versions of NDV F and NDV HN antigens were given the plasmid numbers pMYT1737 and pMYT1742, respectively.

Expression constructs described above were used to transform protease-deficient C1 strains DNL155 and DNL157, from which fourteen protease-encoding genes have been deleted. Each of the expression constructs pMYT1358, pMYT1359, pMYT1360, pMYT1361, pMYT1362, pMYT1363, pMYT1364, and pMYT1365 was used together with a mock vector partner pMYT1140, which is needed for completion of the hygromycin resistance marker gene and integration to the bgl8 locus. Prior to transformation, expression constructs were digested with MssI. The transformations were done with protoplast/PEG method and transformants were selected for nia1+phenotype and hygromycin resistance.

Expression constructs pMYT1737 and pMYT1742 were used together with the mock vector pMYT1729. The mock vector provided a second part of split amdS marker, and a flanking sequence required for correct integration into C1 bgl8 locus. Expression vector pMYT1736 was used in combination either with the mock vector pMYT0781 or with the expression vector pMYT1738. Expression vector pMYT1741 was used in combination either with the mock vector pMYT0781 or with the expression vector pMYT1743. Combination of two split-marker vectors was required to provide both parts of the amdS selective marker and flanking sequences for correct integration into C1 bgl8 locus. Combinations of pMYT1737 and pMYT1742 with pMYT1729, and of pMYT1736 and pMYT1741 with pMYT0781 provided a single copy of the expression cassette, whereas combinations of pMYT1736 with pMYT1738 and of pMYT1741 with pMYT1743 provided two copies of expression cassettes that were placed in opposite (tail-to-tail) directions upon integration into C1 genome. Prior to transformation, all expression constructs were digested with MssI. The transformations were done with protoplast/PEG method and transformants were selected for their ability to grow on selective minimal medium with acetamide as a sole nitrogen source.

Obtained transformants were streaked onto selective medium plates with appropriate selection (either hygromycin resistance or utilization of acetamide) and inoculated from the streaks to liquid cultures in 24-well plates. The medium components were (in g/L) glucose 5, yeast extract 1, (NH₄)₂SO₄ 4.6, MgSO₄·7H₂O 0.49, KH₂PO₄ 7.48, and (in mg/L) EDTA 45, ZnSO₄·7H₂O 19.8, MnSO₄·4H₂O 3.87, CoCl₂·6H₂O 1.44, CuSO₄·5H₂O 1.44, Na₂MoO₄·2H₂O 1.35, FeSO₄·7H₂O 4.5, H₃BO₄ 9.9, D-biotin 0.004, 50 U/ml Penicillin and 0.05 mg Streptomycin. The 24-well plates were incubated at 35° C. with 800 RPM shaking for four days. Culture supernatants were collected and analysed by Western blotting performed with standard methods with the primary detection agents Capture Select Biotin Anti-C-tag conjugate (THERMOFISHER) and chicken NDV antiserum (CHARLES RIVER LABORATORIES), and the secondary agents IRDye 800CW Streptavidin (LI-COR) and IRDye 680LT Donkey anti-Chicken Secondary Antibody (LI-COR). Positive clones producing the target NDV antigens have been identified by Western analysis (FIGS. 5 and 8).

Transformants producing NDV F and HN antigens were purified by single colony plating, and purified clones were verified by PCR for correct integration of the expression cassette and for clone purity. Verified transformants were stored as glycerol stocks at −80° C.

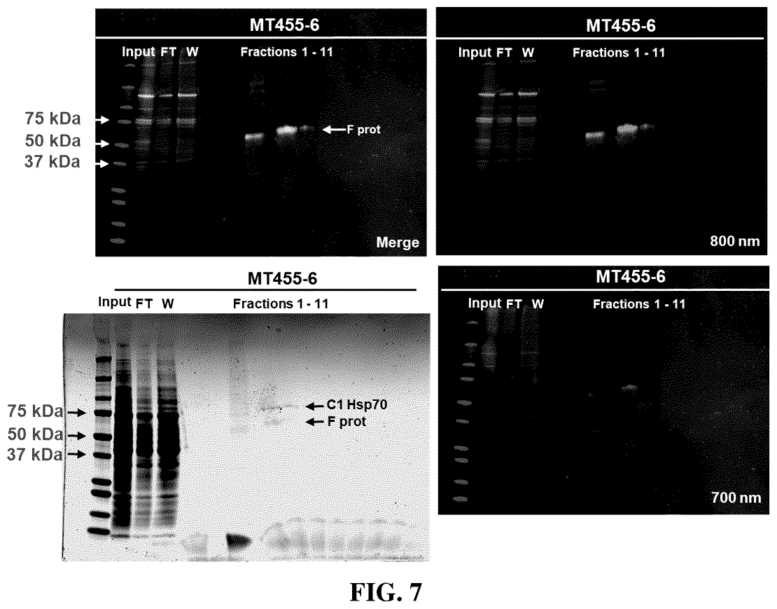

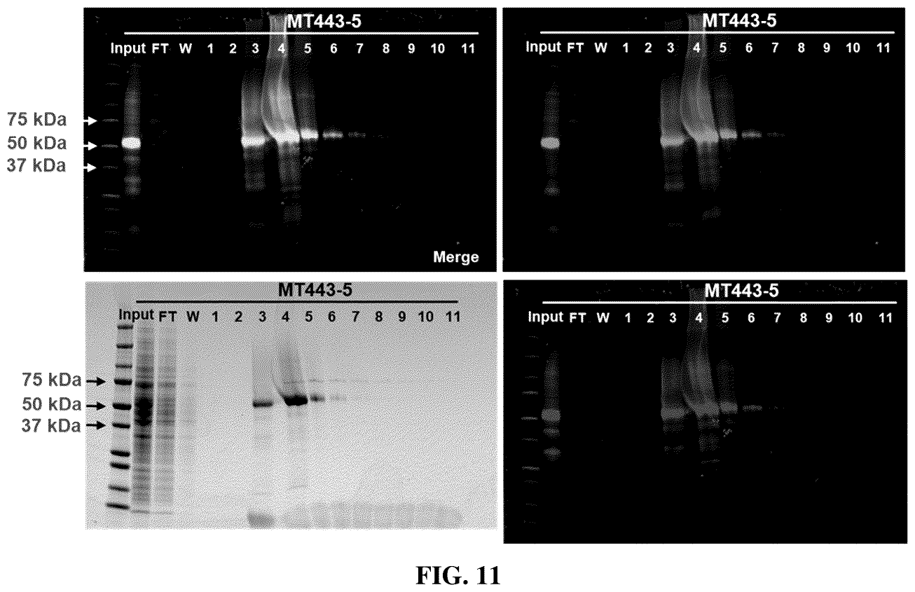

Selected C1 strains producing different forms of NDV F and HN antigens were cultivated in 1 L bioreactor in a fed-batch process in a medium with yeast extract as an organic nitrogen source and glucose as a carbon source. The culture was performed for seven days at 38° C. for HN-producing strains and either at 38° C. or at 25° C. for F-producing strains. After ending the cultivation, fungal mycelium was removed by centrifugation at 4000×g for 20 minutes, serine protease inhibitor PMSF was added to the final concentration 1 mM, and the obtained liquid culture supernatant was stored at −80° C. For purification of target antigens by C-tag affinity chromatography, frozen supernatant was thawed on ice, and after thawing the sample was clarified by centrifugation 3×20 min at 20000×g at +4° C. followed by filtration through a 0.4504 filter. Cleared supernatant was diluted with two volumes of 1×PBS/0.5M NaCl solution (12 mM Na₂HPO₄, 3 mM NaH₂PO₄, 650 mM NaCl pH 7.3). The C-tag affinity purification was performed with 1 ml CaptureSelect C-tag XL column (THERMOFISHER) attached to ÄKTA Start protein purification system (CYTIVA) and operated with a flow rate of 1 ml/min. Column was first equilibrated with 5 column volumes (CV) of 1×PBS/0.5 M NaCl prior loading the sample. After sample loading, the column was washed with 15 CV of 1×PBS/0.5 M NaCl and then eluted with one-step gradient of 10 CV of 20 mM Tris-HCl, 2 M MgCl₂, 1 mM EDTA pH of 7.5 with fraction volume of 1 ml. The amount of the eluted F and HN antigens were quantified by integrating the UV trace of the elution peak with the Unicorn 1.0 software included in the ÄKTA Start system. The extinction coefficients of 0.488 and 0.924 were used in calculating amount of F and HN antigens, respectively. After elution, the column was regenerated with 5 CV of 0.1 M glycine pH 2.3 and washed with 1×PBS until a pH of 7.3 was reached. Elution fractions containing the target protein were pooled for dialysis step to exchange the elution buffer to 1×PBS buffer. Pooled fractions were packed in a 12 ml dialysis cassette and the dialysis cassette was dialyzed in 1.5 L in 1×PBS for 1 hour at +4° C. with stirring on a magnetic stirrer. 1×PBS was exchanged to fresh buffer after 1 h and dialysis was continued for 2 hours under the same conditions. Finally, 1×PBS was exchanged and dialysis was continued overnight. Dialyzed samples were filtered through 0.45 μm filter. Concentration of dialyzed NDV F and HN antigens was determined with the Nanodrop spectrophotometer measuring absorbance at 280 nm and using extinction coefficients 0.488 and 0.924, respectively. Aliquots (1 ml) of antigen preparates were stored at −80° C. C-tag affinity purification of recombinant NDV F and HN antigens is illustrated in FIG. 7 and FIG. 11, respectively.

TABLE 2
Summary of Constructs Used

| | | SIZE OF | | |
|---|---|---|---|---|
| | | TARGET | | |
| | | ANTIGEN | | SELECTION |
| CONSTRUCT | CONTAINS | (KDA) | PROMOTER | MARKER |
| pMYT1358 | CBH1ss-F-protein | 55 | C1 bgl8pr | HygR |
| | (32-553)-Ctag | | | |
| pMYT1359 | CBH1ss-F-protein | 50 | C1 bgl8pr | HygR |
| | (32-499)-Ctag | | | |
| pMYT1360 | CBH1ss-F-protein | 38.5 | C1 bgl8pr | HygR |
| | (146-499)-Ctag | | | |
| pMYT1361 | CBH1ss-F-protein | 50 | C1 bgl8pr | HygR |
| | (32-499, K115G)-Ctag | | | |
| pMYT1362 | HN protein (1-571)- | 63 | C1 bgl8pr | HygR |
| | Ctag | | | |
| pMYT1363 | CBH1ss-HN protein | 63 | C1 bgl8pr | HygR |
| | (1-571)-Ctag | | | |
| pMYT1364 | CBH1ss-HN protein | 55 | C1 bgl8pr | HygR |
| | (78-571)-Ctag | | | |
| pMYT1365 | CBH1ss-HN protein | 50 | C1 bgl8pr | HygR |
| | (124-571)-Ctag | | | |
| pMYT1736 | CBH1ss-F-protein | 50 | C1 bgl8pr | amdS |
| | (32-499, K115G)-Ctag | | | |
| pMYT1737 | CBH1ss-F-protein | 50 | AnSESpr | amdS |
| | (32-499, K115G)-Ctag | | | |
| pMYT1738 | CBH1ss-F-protein | 50 | C1 bgl8pr | amdS |
| | (32-499, K115G)-Ctag | | | |
| pMYT1741 | CBH1ss-HN protein | 50 | C1 bgl8pr | amdS |
| | (124-571)-Ctag | | | |
| pMYT1742 | CBH1ss-HN protein | 50 | AnSESpr | amdS |
| | (124-571)-Ctag | | | |
| pMYT1743 | CBH1ss-HN protein | 50 | C1 bgl8pr | amdS |
| | (124-571)-Ctag | | | |

### Example 2

### Evaluation of Efficacy and Safety for the Inactivated Newcastle Disease Virus Antigens (Produced in C1 Technology) in Specified Pathogen-Free Chickens

### Study Approval and Ethics

Approval for this trial was obtained from the Israeli National Animal Use and Care Committee before the trial commenced. Compliance with this protocol assured that due regard was given to the welfare of the birds enrolled in this study, the protection of the personnel involved in this study, and the environment and human food chain.

This trial was performed in accordance with the European Pharmacopoeia requirements—Ph.Eur, Edition 10.0, and monograph number 01/2017:0870 subtitle 2-4-2-1 Evaluation of efficacy of veterinary vaccine and immunosera, and in accordance with the European Guidelines on the design of studies to evaluate the safety and efficacy monograph 07/2018:0062, subtitle 20402.

### Background

Newcastle Disease (ND) is caused by virulent strains of avian paramyxovirus type-1 and is also referred to as Newcastle Disease virus (NDV). NDV has a marked genomic variability. Currently, genotypes V, VI, VII, and VIII (from class II) are predominant globally. An inactivated NDV vaccine (NECTIV FORTE) is routinely used to vaccinate chickens and turkeys against ND. The vaccine provides slow release but a longer duration of immunity. The vaccine formulation is a water-in-oil emulsion with a mineral oil base and two emulsifiers, MONTANE and MONTANOX. To produce this vaccine, approximately two billion embryonic chicken eggs are used a year to propagate the needed antigens. This process is costly and labor intensive.

Embodiments of the present disclosure produce NDV vaccination components without having to sacrifice the billions of chicken embryos currently needed. The specific objective of this example was to evaluate the immunogenicity and protection provided by the NDV antigens produced in the C1 systems as compared to the use of a commercially available inactivated vaccine, as well as a negative control treatment.

### Study Design

This trial was conducted to evaluate the immune response of novel NDV vaccines formulated with HN and F proteins produced in the C1 fungal expression system. The new vaccine formulations are based on the inactivated antigens in oil emulsions for intramuscular vaccination. Six different formulations will be tested as described in Table 3. The test vaccines were prepared in ABIC R&D laboratory. Specified pathogen free (SPF) chickens were used, and each was given two intramuscular vaccinations. The treated chickens were then challenged with vvNDV G7.

TABLE 3
Summary of Vaccine Formulas

| | | | | DOSE | CHICKENS |
|---|---|---|---|---|---|
| | | | ROUTE OF | VOLUME | PER |
| GROUP | FORMULA | BATCH NO. | VACCINATION | (ML) | GROUP |
| 1 | Negative | 2048951 | IM | 0.5 | 10 |
| | control (PBS) | | | | |
| 2 | NECTIV | A0022111672 | IM | 0.3 | 10 |
| | FORTE | | | | |
| 3 | HN63 | M-1-2642 | IM | 0.5 | 10 |
| 4 | HN55 | M-1-2643 | IM | 0.5 | 10 |
| 5 | HN50 | M-1-2644 | IM | 0.5 | 10 |
| 6 | F50 | M-1-2645 | IM | 0.5 | 10 |
| 7 | HN63 + F50 | M-1-2626 | IM | 0.5 | 10 |

In this trial, the first vaccination was completed in SPF chickens at 15 (±3) days of age. The second booster vaccination was completed three weeks later when the chickens were then five weeks of age. Throughout the trial, antibody response was monitored via serological methods (HI test). Three weeks after the second vaccination, the subjects were challenged with vvNDV-G7 (358/2015 strain IVS).

To evaluate the efficacy, zero-time bleeding from ten chickens was done on the same day, but just prior to, the first vaccination. Bleedings from all chickens were done before the second vaccination at five weeks, and at three weeks after the second vaccination (eight weeks). These bleedings were completed prior to the challenge with vvNDV. These steps are summarized in Table 4. The sera collected were used for an HI serology test and determined the immunogenicity of the tested vaccines. The challenge with the vvNDV determined the level of protection provided by the vaccines. The animal selection and identification information are provided in Table 5.

TABLE 4
Action Item Timeline

| TRIAL DAY | DAY AFTER | |
|---|---|---|
| (±3 DAYS) | VACCINATION | ACTION COMPLETED |
| 1 | — | Hatching and moving to the inactivated |
| | | unit |
| 15 | 0 | Wing numbering |
| 15 | 0 | Zero-time bleeding from ten chickens |
| 15 | 0 | Prime vaccination |
| 35 | 20 | First bleeding |
| 35 | 20 | Boost vaccination |
| 55 | 40 | Second bleeding |
| 57 | 42 | Challenge with vvNDV Genotype VII |
| | | (strain 358/2015 IVS) |
| 71 | 56 | End of challenge monitoring and end |
| | | of trial |

TABLE 5
Details of Animal Subjects

| | |
|---|---|
| Species | One day old SPF chicks-Gallus spp. white leghorn breed was |
| | hatched from SPF eggs from an SPF flock |
| Supplier Flock No. | Animal House: SPF eggs purchased from Supplier: SPAFAS Batch |
| | No. 032932-1697 |
| Initial Age | One day old SPF chickens |
| Serological Status | The Certificate of Analysis of the SPF embryonated eggs used |
| | attached to the report |
| Sex | Mixed |
| Origin | Embryonated SPF eggs will be incubated within the SPF hatchery at |
| | Phibro's Test Facility (according to WI-LV-004) until enrollment in |
| | the study at 18 days old embryo. Then, the well-developed embryos |
| | will be transferred to the Test Facility Test Site (with filled and signed |
| | QCM-190Form01). Upon arrival to the Test Facility Test Site, the |
| | eggs will be placed into the hatchery according to AH-016. |
| Hatching | According the AH-001 and QCM-190Form01: Hatching the |
| | embryonated SPF and transfer to the isolator unit. SPF chickens will |
| | be from the same flock and batch of eggs. From the beginning |
| | through the duration of the study, the birds will be subjected to daily |
| | inspection of the animal care and welfare office for the daily records. |
| | Only chickens showing normal behavior were selected for the study. |

### Animal Subjects

Only healthy chicks were included in this study. During the study, sick or dead birds were thoroughly examined by a veterinarian and the cause of the event was recorded. If the cause of the event was unrelated to the vaccination, the birds were excluded from the analysis of the results.

Both male and female one-day old chicks intended for treatment groups 1-7 were paced in a cardboard box. The chicks were removed randomly and placed into the rooms for vaccinations. Each chick was uniquely identified by wing tagging at the day of the vaccination according to AH-001. Wing tag numbers and treatment groups were recorded.

Table 6 summarizes the animal management and housing.

TABLE 6
Summary of Animal Management

| | |
|---|---|
| ALL MANAGEMENT AND | ALL ANIMAL STUDIES FOR NDV WERE |
| HUSBANDRY PROCEDURES | PERFORMED IN LINE WITH THE RELEVANT |
| WERE IN ACCORDANCE | LEGISLATION IN EU (DIRECTIVE 2010/63/EU) |
| WITH AH-001 AND AH-001 | AND ISRAEL, AND WITH ISRAELI ANIMAL |
| ANIMAL WELFARE | WELFARE LEGISLATION. |
| Housing | Study animals were allocated in the poultry house |
| | rooms for inactivated vaccines for the whole period of |
| | the study, in Phibro's Test Facility test site at Har-Tuv. |
| | Prior to the challenge, the chicks were transferred to |
| | KVI for the velogenic NDV challenge |
| Feed and water | Tap water and food were available ad libitum. |
| | Feedings were of the standard rations appropriate to the |
| | age of the birds and used standard chick diet (Tadmir |
| | 3210, Israel). Water was provided by drinking nipple. |
| Permissible and non-permissible | No treatment other than the NDV vaccination was |
| concomitant veterinary care and | administered during the study. The use of feed formula |
| therapy | additives did not impact the study (MTF-078). No |
| | antibiotic treatments were added during the period the |
| | of trial. |
| Environmental control | Environmental control, including room temperature, |
| | was recorded daily. |
| Rearing of study animals | Study animals were maintained at temperatures within |
| | the guidelines for the age of bird accordioning to AH- |
| | 001 using built-in heaters with thermostats. |

### Tested Vaccines

The vaccines used are described in Table 7. The source of the protein antigens for the HN and F proteins is described in Table 8.

TABLE 7
Description of Vaccine or Antigen Used in Vaccine

| INACTIVATED | |
|---|---|
| VACCINE | DESCRIPTION |
| HN63 | Group No. 3; full length HN protein (63 kDa) |
| | (SEQ ID NO: 22) |
| HN55 | Group No. 4; truncated HN protein (55 kDa) |
| | (residues 78-571, SEQ ID NO: 25) |
| HN50 | Group No. 5; truncated HN protein (50 kDa) |
| | (residues 124-571, SEQ ID NO: 28) |
| F50 | Group No. 6; truncated F protein with modified |
| | protease cleavage site (50 kDa) (residues 32-499, |
| | substitution K115G; SEQ ID NO: 16) |
| HN63 + | Group No. 7; mixtures of the full length HN |
| | protein (SEQ ID NO: 22) and the truncated |
| F50 | modified F protein (residues 32-499, SEQ ID NO: 16) |
| NECTIV | Group No. 2; batch no. A0022111672, commercial |
| | inactivated vaccine based on VH strain (NDV |
| FORTE | vaccine strain, genotype 2) |

TABLE 8
Antigen Source for Vaccine Formulations

| | | | | CONCENTR | |
|---|---|---|---|---|---|
| | | | | ATION OF | AMOUNT OF |
| | | | PRODUCTION | PURIFIED | PURIFIED |
| | | | LEVEL | SAMPLES | PROTEIN |
| PROTEIN | CONSTRUCT | FERM. | (MG/ML) | (MG/ML) | (MG) |
| HN63 | pMYT1363 | MT441 | 0.94 | 0.6 | 7.2 |
| HN55 | pMYT1364 | MT442 | 0.62 | 0.5 | 6.0 |
| HN50 | pMYT1365 | MT443 | 1.13 | 0.85 | 13.6 |
| F50 | pMYT1361 | MT461 | 0.3 | 0.65 | 7.15 |

### Vaccine Preparation

Preparation of Small-Scale Tested Batches

The protein concentration was adjusted to 50 μg per 0.5 mL per vaccine dose. Each chicken received two doses during the trial—a prime and a boost vaccination. The emulsion preparation was done for both vaccinations. After preparation the vaccines were filled into sterile tubes and stored at 2-8° C. until use.

Materials:
1. Mineral oil, e.g., DRAKEOL, (Supplier: Gadot; lot no.: 10-035431)
2. Sorbitan oleate, an adjuvant (Supplier: Zefroni sapaki chmicalim Ltd.; lot no.: 10-038020)
3. PBS (Supplier: Biological Industries Ltd.; lot no.: 2048951)
4. Sorbitol-T, an adjuvant (Supplier: Zefroni sapaki chmicalim Ltd.; lot no. 10-035482)

Preparation:
1. In a sterile laminar flow hood, each formula was prepared separately.
2. The oil phase was composed of a mineral oil, e.g., DRAKEOL, and an emulsifier, e.g., MONTANE.
3. In a different tube, the water phase (at 30%) was prepared from each antigen, sterile PBS, and an emulsifier, e.g., MONTANOX.
4. The oil phase was mixed in the homogenizer on slow speed, and the water phase was poured into the oil phase until it was homogenized. The speed was then increased, and the solution was mixed for 60 seconds. Table 9 presents the volume of components in the final emulsions.
5. Each volume was then divided into two equal portions in 15 mL tubes.
6. The tubes were closed, and the labels were double checked.
7. Prepared final emulsions in tubes were then stored at 2-8° C. until use.

TABLE 9
Component Volumes Used in Vaccine Formulas

| | VOLUME OF COMPONENT ADDED (ML) PER | | | | |
|---|---|---|---|---|---|
| | VACCINE FORMULA | | | | |
| | | | | | HN63 + |
| COMPONENT | HN63 | HN55 | HN50 | F50 | F50 |
| DRAKEOL (67%) | 10 | 10 | 10 | 10 | 10 |
| Sorbitan oleate (3%) | 0.45 | 0.45 | 0.45 | 0.45 | 0.45 |
| Antigen* | 3.75 | 3.2 | 2.1 | 2.9 | 3.75 + 2.9 |
| PBS | 0.65 | 1.2 | 2.3 | 1.5 | — |
| Sorbitol-T (1%) | 0.15 | 0.15 | 0.15 | 0.15 | 0.15 |
| Total Volume (mL) | 15 | 15 | 15 | 15 | 17.25 |

*Antigen concentrations (mg/mL): HN63 = 0.4; HN55 = 0.48; HN50 = 0.73; F50 = 0.52

Inactivated Vaccine

A commercial inactivated ND (VH strain) vaccine, NECTIV FORTE, batch No. A0022111672, was used as a reference for efficacy evaluation. Each chick from Group No. 2 was vaccinated with 0.3 mL of the NECTIV FORTE vaccine by IM route into pectoralis muscle.

### Administration

Vaccinations via IM into the pectoralis muscle were completed according to the timeline as described in Table 4.

### Challenge Virus

The challenge was performed in KVI using the genotype VII vND (isolate 358/2015 IVS) with a dose of 10^{5.3} EID50 per a volume of 0.5 mL by IM injection.

### Assessment of Efficacy

Parameters for Evaluation

Serology for the sera was evaluated by HI method for recombinant LaSota-HN-G7 strain developed in the R&D department. Antibody titers induced by NDV were tested following the two vaccinations. Comparisons were made the commercial vaccine NECTIV FORTE and the five formulations of the novel vaccinations. All samples of sera were evaluated using a calibrated antigen of the homologous VH and laSota-HN-G7 viruses.

Test Limits:

HI negative control should be below 2 units.

HI positive are samples that have 2 unites above the negative control.

All relevant sera were tested as well by ELISA to detect F protein antibodies using the NDV-F ELISA by Biocheck. The test limits were according to the commercial kits' instructions.

Viral Challenge

Protection levels were evaluated for all vaccinated groups with the commercial vaccine NECTIV FORTE and the five formulations of the new vaccines following challenge with the velogenic ND virus Genotype VII (358/2015 IVS). The test was valid as mortality in the negative control treatment was 100% post-challenge.

### Results

Titers

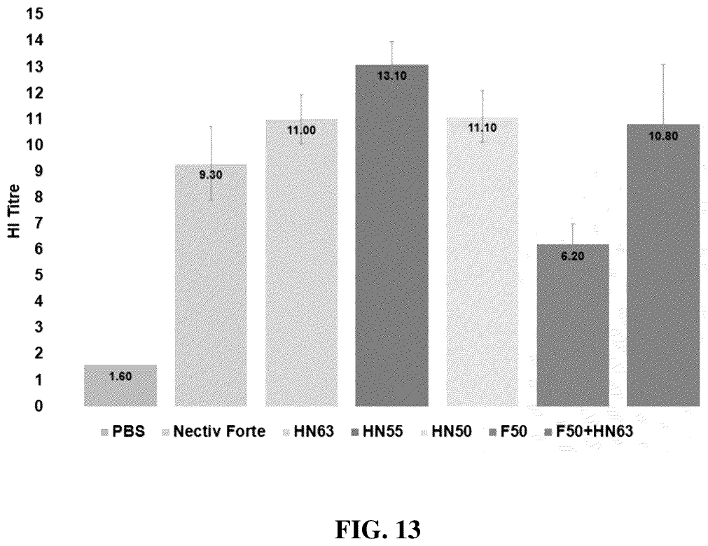

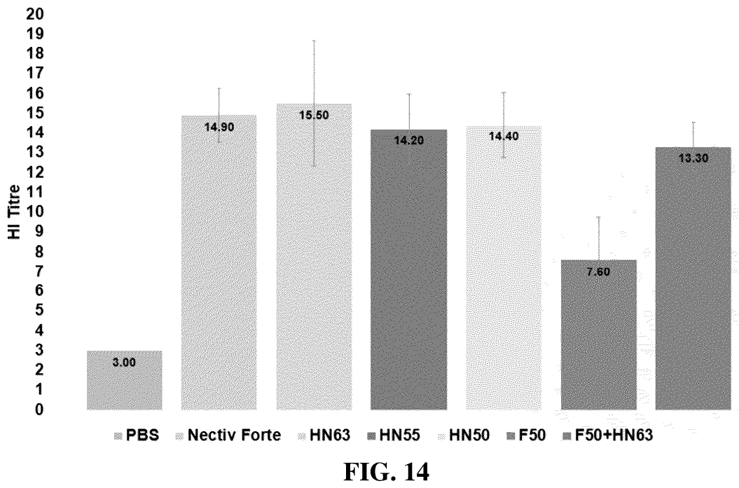

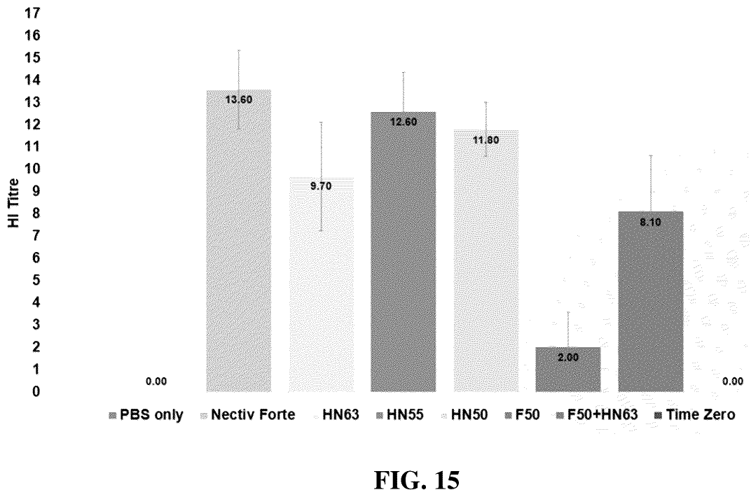

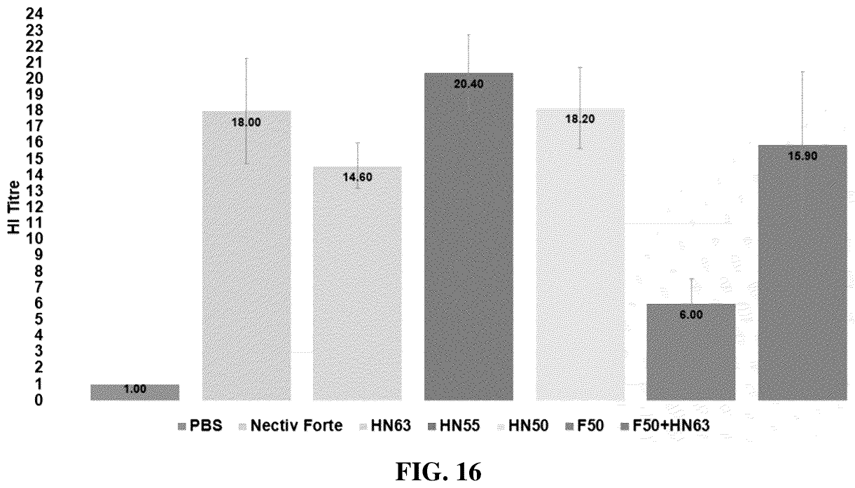

For HI titers against genotype 7 antigen, all birds' sera from the first bleeding were tested with rLaSota-HN-G7 antigen. In this test, 24 serial dilutions were included to have the maximal HI potential. Results are shown in FIG. 13. The birds' sera from the second testing were also tested with the rLaSota-HN-G7 antigen in a 24 serial dilution format for HI potential for titers against genotype 7. The results from the second testing are shown in FIG. 14. Titers against genotype 2 after the first dose were tested with VH antigen and shown in FIG. 15. The second dose response was also measured by HI titers against genotype 2 (VH) antigen and the results are shown in FIG. 16.

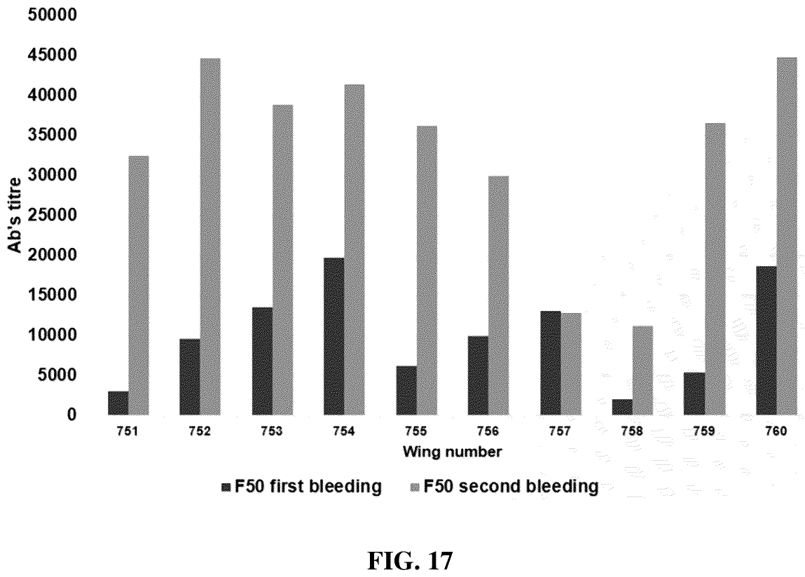

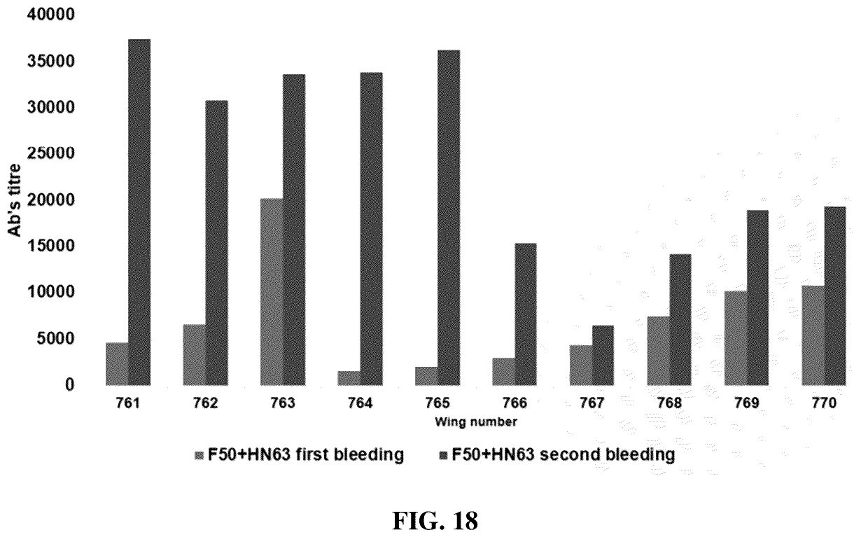

For ELISA-NDV-F titers, the F50 treatment group birds' sera were tested on NDV-F ELISA. The test was valid, and the results are show in FIG. 17. Finally, the ELISA-NDV-F titers for the HN63+F50 treatment group are shown in FIG. 18. This test was also valid.

The HI titers are summarized in Table 10.

TABLE 10
HI Titers of Vaccines Used

| | LASOTA HN STRAIN-G7 | | VH STRAIN-G2 | |
|---|---|---|---|---|
| VACCINE | First | Second | First | Second |
| TREATMENT | bleeding | bleeding | bleeding | bleeding |
| PBS | 1.6 | 3 | 0 | 1 |
| NECTIV FORTE | 9.3 | 14.9 | 13.6 | 18 |
| HN63 | 11.0 | 15.5 | 9.7 | 14.6 |
| HN55 | 13.1 | 14.2 | 12.6 | 20.4 |
| HN50 | 11.1 | 14.4 | 11.8 | 18.2 |
| F50 | 6.2 | 7.6 | 2 | 6 |
| F50 + HN63 | 10.8 | 13.3 | 8.1 | 15.9 |

Protection

After challenge with the velogenic NDV Genotype VII (358/2015 IVS), mortality of subjects in the treatment groups was recorded to determine the level of protection offered by the various vaccines, as summarized in Table 11.

TABLE 11
Protection Levels of the Vaccines Tested

| | | PERCENT |
|---|---|---|
| VACCINE TREATMENT | DECEASED/TOTAL | PROTECTION |
| GROUP | SUBJECTS | LEVEL |
| PBS | 10/10 | 0% |
| NECTIV FORTE | 1/10 | 90% |
| HN63 | 0/10 | 100% |
| HN55 | 0/10 | 100% |
| HN50 | 1/10 | 90% |
| F50 | 1/10 | 90% |
| HN63 + F50 | 0/10 | 100% |

Conclusions

All new recombinant HN antigens generated high levels of antibodies against both the genotype 7 and genotype 2 NDV strains. In addition, the recombinant F antigen generated high levels of antibodies as well. The vaccinated chickens were found to be resistant in challenge with velogenic NDV Genotype VII (358/2015 IVD). The mixture of HN63 with F50 did not provide added value in serology or challenge test. All recombinant antigens were found to be equivalent to the positive control inactivated vaccine.

### Example 3

### PD50 and Reduction in Challenge Virus Secretion Evaluation of Inactivated HN50 and F50 Recombinant Vaccines (Produced in C1 Technology) in SPF Chickens

### Background

Newcastle Disease (ND) is a highly contagious viral disease that can affect all bird species. It is ranked as the fourteenth most problematic disease of poultry and the eighth cause of wild animals lost through destruction. This enveloped virus (Avulavirus genus, Paramyoxoviridae family, Mononegavirales order, recently renamed as Avian Avulavirus or AAvV) has a marked genomic variability and the genotypes V, VI, VII and VIII (from class II) are predominant in different countries all over the world.

Inactivated ND vaccine (NECTIV FORTE) is used routinely to vaccinate chickens and turkeys against ND. The vaccine provides slow response but longer duration of immunity. The vaccine formulation is a water in oil emulsion with emulsifiers, where the oil is a mineral oil, and emulsifiers are MONTANE and MONTANOX. This vaccine is dependent on producing viral antigens in embryonic chicken eggs which is time consuming and labor intensive. While other commonly used expression systems have failed to produce properly folded or biologically active antigens, C1 is herein disclosed as a compatible expression system to produce biologically active NDV antigens in a more efficient manner than use of embryonic chicken eggs.

C1 has a unique morphology allowing for hyper productivity and growth under low viscosity using a broad range of fermentation conditions at flexible commercial scales. This expression system provides high-yield, low cost, rapidly scalable, rVaccine and antibody gene expression platform in single use or stainless-steel bioreactors. It offers safe and effective antigens, generally recognized as safe (GRAS) certification from US FDA, and no negative clinical effects have been seen in animal studies. In addition, the native C1 glycan structure is more mammalian-like than that of yeast. Native C1 glycans are mostly high mannose type (Man3-Man9) including low amount of hybrid glycans. Example 2 demonstrated the ability of three recombinant HN protein antigens and one F protein antigen to confer high Ab's levels with good protection levels in vvNDV challenge in poultry.

The purpose of this example is to demonstrate the PD50 of the recombinant HN50 and F50 proteins produced in C1 and used in inactivated vaccines. Specifically, this example shows the protective dose and evaluates the amount of PD50 that exists in a volume of 0.5 mL of vaccine (50 μg recombinant protein per dose). This example also establishes the correction between the protective dose, serological responses, and percentage of protection. Finally, the reduction in NDV genotype VII excretion after viral challenge is also demonstrated.

### Justification

The Test System described below is based on the requirements of the European Pharmacopoeia (Ph. Eur) 10.5, Newcastle Disease Vaccine (Inactivated) 07/2020:0870: “2-3-2-1. Vaccines for use in chickens.

However, considering the strain found in Israel, the NDV challenge was carried out with vNDV gVII (strain 358, recently isolated). The challenge was performed with a dose of 10^{6.0} ELD₅₀, respectively, by IM injection 21 days post vaccination. The chickens were observed at least daily for 21 days after challenge and the deaths and the number of surviving chickens that show clinical signs of disease were recorded.

### Study Design

Treatment Groups

195 SPF chicks were hatched in Har-Tuv animal house and separated into 13 groups. All groups had 15 birds each. Each group was placed in cages in the inactivated room and vaccinated with the inactivated vaccines.

On day 21, chicks from groups 1-13 were wing numbered and vaccinated by intramuscular (IM) route, with the different volume of doses of the tested vaccines as follows for each chick (summarized in Table 12): groups 4 and 9 received a dose of 1:200 of its respective vaccine, groups 5 and 10 received a dose of 1:100 of its respective vaccine, groups 6 and 11 received a dose of 1:50 of its respective vaccine, groups 7 and 12 received a dose of 1:25 of its respective vaccine, and groups 8 and 13 received a dose of 1:12.5 of its respective vaccine (Table 12). Chicks from group 1 were not vaccinated and were kept as a non-vaccinated control group. Chicks from groups 2 and 3 received the full dose of F50 and HN50, respectively.

On 21 days post-vaccination, all chicks were bled, and the HI titers were determined using the HI method BSH-QCM-0000626 (E-QCM-169-03), with VH antigen and the rNDV-LS-HN G7 antigen by the European Pharmacopoeia (EP), Edition 10.5, monograph (number 07/2020:0870, subtitle 2-5-2-3, Haemagglutination inhibition).

After 21 days (3 weeks) post-vaccination, all groups, from 1 to 13, were sent to the Kimron Veterinary Institute (KVI) for challenge by IM route with a dose of 10^{6.0} ELD50 of vNDV genotype VII (strain 358). All the chicks were observed during at least 21 days after challenge.

4, 6 and 8 days after challenge tracheal and cloacal swabs were collected from groups 1-5, 9-10 and were submitted for viral RNA quantification using qPCR method.

At the end of the observation period after the challenge, the PD50 were calculated by using statistical analysis comparing the number of surviving birds in each group without any sign of Newcastle Disease. Table 12 summarize the trial procedure. Blood samples were taken before vaccination (time zero) and before challenge for serology tests.

TABLE 12
Study Groups and Stages of Activities

| | | | Bleeding | | Dose | Injection | | |
|---|---|---|---|---|---|---|---|---|
| | | | before | | (Injected | volume | | |
| | | | vaccination | | volumes | (μL) | Bleeding | Challenge |
| | Tested | No. of | age | Vaccination | of the | by IM | before | Age |
| Group | items | chickens | (days) | age (days) | vaccines) | route | challenge | (days) |
| 1 | Control | 15 | 21 | — | — | — | 41 | 42 |
| | Group | | | | | | | |
| 2 | F50 | 15 | 21 | 21 | 1.0 | 500 μL | 41 | 42 |
| 3 | HN50 | 15 | 21 | 21 | 1.0 | 500 μL | 41 | 42 |
| 4 | F50 | 15 | 21 | 21 | 1:12.5 | 40 μL | 41 | 42 |
| 5 | | 15 | 21 | 21 | 1:25 | 20 μL | 41 | 42 |
| 6 | | 15 | 21 | 21 | 1:50 | 10 μL | 41 | 42 |
| 7 | | 15 | 21 | 21 | 1:100 | 5 μL | 41 | 42 |
| 8 | | 15 | 21 | 21 | 1:200 | 2.5 μL | 41 | 42 |
| 9 | HN50 | 15 | 21 | 21 | 1:12.5 | 40 μL | 41 | 42 |
| 10 | | 15 | 21 | 21 | 1:25 | 20 μL | 41 | 42 |
| 11 | | 15 | 21 | 21 | 1:50 | 10 μL | 41 | 42 |
| 12 | | 15 | 21 | 21 | 1:100 | 5 μL | 41 | 42 |
| 13 | | 15 | 21 | 21 | 1:200 | 2.5 μL | 41 | 42 |

### Schedule of Events

TABLE 13
Trial Activities and Schedule of Events

| TRIAL DAY | ACTION |
|---|---|
| 1 | Hatching of SPF chicks. |
| 21-28 | Wing tag. |
| | Bleeding for serology time zero. |
| | Vaccination according to table 1. |
| 25-32 | Veterinary inspection for vaccination clinical signs |
| | evaluation. (According to AH-004Form02) |
| 41-48 | Bleeding for serology. |
| | All groups were transferred to the veterinary services for |
| | challenge with vNDV genotype VII (strain 358). |
| 42 | Challenge at the veterinary services. |
| 46 | Tracheal and cloacal swabs from groups 1-5, 9-10 |
| 48 | Tracheal and cloacal swabs from groups 1-5, 9-10 |
| 50 | Tracheal and cloacal swabs from groups 1-5, 9-10 |
| 42-63 | Daily observation and recording of morbidity and mortality. |
| 63 | Euthanasia of the chickens and end of the trial. |

### Materials

Test System—Animals

All management and husbandry procedures were according to AH-001. Table 14 shows details about the test system used in this study.

TABLE 14
Detailed Information of The Test System Performed

| | |
|---|---|
| SPECIES: | DAY OLD SPECIFIED PATHOGEN FREE (SPF) CHICKS - |
| | GALLUS SPP. WHITE LEGHORN BREED WAS HATCHED |
| | FROM SPF EGGS FROM A SPF FLOCK. |
| Supplier flock | M104; Lot no. 032932-1771 |
| number | |
| Initial Age | One day old chickens at day 0 (zero) |
| Serological Status | The COA of the SPF embryonated eggs used, were attached to the |
| | report. |
| Sex | Male or female |
| Origen | Embryonated SPF eggs were sourced from a commercial supplier |
| | and incubated within the SPF hatchery at Phibro's Test Facility |
| | (according to WI-LV-004) until enrolment onto the study at 18 days |
| | old embryo. Then, the well-developed embryos were transferred to |
| | Har tuv (with filled and signed QCM-190Form01). Upon arrival to |
| | Har tuv, the eggs were placed into the hatchery according to AH-016. |
| Hatching | According to AH-001 and QCM-190Form01 hatching the |
| | embryonated SPF and transfer to the inactivated room. SPF |
| | chickens were from the same SPF flock and batch of eggs. From the |
| | beginning through the duration of the study the birds were |
| | subjected to daily inspection of the animal care and welfare office |
| | for the daily records (according to the AH-004Form02). Only |
| | chickens showing normal behavior (according to AH-004Form02) |
| | were selected for the study |

Inclusion/Exclusion/Withdrawal Criteria

To be included in this study, chicks had to satisfy the following requirements: (1) originate from the same SPF flock and batch of eggs, and (2) be healthy chicks free from signs of disease upon enrolment as determined by normal appearance and normal behavior.

Acceptance of birds into the study was recorded into AH-004Form04. Daily observation was performed for 21 days following challenge by the Veterinary Services of Ministry of Agricultural and Rural Development. During the study, any sick or dead birds until the challenge end was examined by the responsible veterinarian surgeon and the cause recorded into AH-004Form04. During the whole period of the study procedures, the animals withdrawing was conducted according to AH-016. The number of animals withdraw prematurely from the studies and the reasons for such withdraw were recorded.

Animal Management

The animal management and husbandry procedures are shown in Table 15.

TABLE 15
Animal Management

| | |
|---|---|
| ANIMAL | WE DECLARE AND GUARANTEE THAT ALL |
| WELFARE | ANIMAL STUDIES FOR HN50 AND F50 |
| | VACCINE WERE IN LINE WITH THE RELEVANT |
| | LEGISLATION IN EU (DIRECTIVE 2010/63/EU) |
| | AND ISRAEL AND INACCORDANCE WITH |
| | ISRAELI ANIMAL WELFARE LEGISLATION. |
| Housing | Study animals were mixed and allocated in cages |
| | in the inactivated room, the whole period of study, |
| | in Phibro's Test Facility - test site at Har Tuv, |
| | until the challenge date. |
| Food and | Tap water and food were available ad libitum. Food |
| water | was in the form of standard rations appropriate to |
| | the age of the birds: Standard chick diet (Tadmir |
| | 3210, Israel) and provided by drinking nipples. |
| Permissible | No treatment other than the F50 or HN50 |
| and non- | vaccination was administered during the study. |
| permissible | Use of additives in food formula has no impact on |
| concomitant | the study (MTF-078). No antibiotic treatment was |
| veterinary | added during the period of the trial. |
| care and | |
| therapy | |
| Environmental | Environmental control was recorded daily in “daily |
| control | check form” AH-004Form02. |
| Rearing of | Study animals were maintained at temperatures |
| study animals | (using built-in heater with thermostat) within |
| | the guidelines for the age of bird (as detailed in |
| | table 3), lightning according to AH-001. |

Housing temperature according to poultry age:

| Temperature | Age in weeks | |
|---|---|---|
| 32 ± 3° C. | 1-4 | days |
| 31 ± 3° C. | 4-7 | days |
| 29 ± 3° C. | 2 | weeks |
| 27 ± 3° C. | 3 | weeks |
| 26 ± 3° C. | 4 | weeks |
| 25 ± 3° C. | 5-7 | weeks |

Animals Randomization

The day-old chicks intended for treatment groups from 1 to 13 were placed in a cardboard box and, from there, removed randomly and assigned to one of the groups. Both male and female were selected for the trial. Each chick was identified by wing tagging at the vaccination day.

Blinding

Only the HI test was blinded. The vaccination was not blinded since the volume for vaccination was different in each group and needed to be familiar to the technician.

### Test Items

Test Item Characterization

TABLE 16
Characteristics of Vaccine Emulsion Preparations

| IDENTITY | F50 | HN50 |
|---|---|---|
| Appearance | w/o emulsion | w/o emulsion |
| Supplier | Abic Biological | Abic Biological |
| | Laboratories | Laboratories |
| Batch number of emulsions | M-1-2670 | M-1-2671 |
| Storage conditions | 4° C. | 4° C. |
| Manufacturing date | Jul. 15, 2021 | Jul. 15, 2021 |
| Dosage | 50 μg/0.5 mL | 50 μg/0.5 mL |

F50 and HN 50 Proteins

To produce the emulsion, the purified F50 and HN50 proteins were not previously inactivated.

Inactivated F50 and HN50 Vaccines

The methods to produce emulsions for the F50 and HN50 proteins are as follows.

F50 (M-1-2670): The water phase was prepared in a sterile hood by diluting 2.88 mL of F50 into 1.52 mL PBS. 0.15 mL of Sorbital T was added, and the mixture was incubated at 37° C. for 30 minutes with occasional mixing/vortexing. 10 mL of mineral oil was mixed with 0.45 mL of Sorbital S. The emulsion was homogenized in a Polytron at maximal speed for 1 minute.

HN50 (M-1-2671): The water phase was prepared in a sterile hood by diluting 2.1 mL of HN50 into 2.3 mL PBS. 0.15 mL of Sorbital T was added, and the mixture was incubated at 37° C. for 30 minutes with occasional mixing/vortexing. 10 mL of mineral oil was mixed with 0.45 mL of Sorbital S. The emulsion was homogenized in a Polytron at maximal speed for 1 minute.

15 mL of emulsion was produced by using Montane (Sorbital-S), Montaneox (Sorbitol-T), mineral oil and PBS as shown into Table 17.

TABLE 17
HN50 and F50 Emulsions Preparations

| | | |
|---|---|---|
| FORMULA | F50 | HN50 |

| | | | | |
|---|---|---|---|---|
| DRAKEOL (67%) | 10 | mL | 10 | mL |
| Montane (3%) | 0.45 | mL | 0.45 | mL |
| *Antigen | 2.88 | mL | 2.1 | mL |
| PBS | 1.52 | mL | 2.3 | mL |
| Montaneox (1%) | 0.15 | mL | 0.15 | mL |

| | | |
|---|---|---|
| Total volume (mL) | 15 | 15 |

*Antigens concentrations: HN50 = 0.73 mg/mL; F50 = 0.52 mg/mL.

Tests Doses

At 21 days of age, all chicks from groups 2 to 13 were IM vaccinated into the pectoral muscle. A 10 μl syringe was used for this vaccination. Each chick from groups 4 and 9 received a dose of 40 μl of its respective vaccine; groups 5 and 10 received a dose of 20 μl of its respective vaccine; groups 6 and 11 received a dose of 10 μl of its respective vaccine; groups 7 and 12 received a dose of 5 μl of its respective vaccine; and groups 8 and 13 received a dose of 2.5 μl of its respective vaccine (Table 12). Groups 2 and 3 received a dose of 500 μl of F50 and HN50 (Table 12). Group 1 and Group 15 chicks were not vaccinated.

At 42 days of age (21 days after vaccination), 15 chicks from all groups 1 to 13 were challenged by IM route, into pectoral muscle. Each chick from the groups 1 to 13 received a dose of 10^{6.0} ELD50/dose of the challenge material (Table 18).

TABLE 18
Challenge Material Characterization

| IDENTITY | VNDV GVII (STRAIN 358) |
|---|---|
| Supplier | Kimron Veterinary Institute (KVI) |
| Titer | 106.0 ELD50/dose |

### Methods

Test Administration Method—Vaccination

For the vaccination, the vaccines were removed from the refrigerator and were warmed using a warm water bath to the vaccines achieve approximately 37-39° C. This procedure reduces the emulsion viscosity, which facilitates administration and prevents local reactions and pain.

The vaccine containers were gently agitated before and during the vaccination process to homogenize the contents. The required amount of the vaccine was manually injected in the chickens using a syringe (volume capacity of 10 μL).

The vaccinations order was performed starting with all chickens that were injected with the inactivated F50 batch number M-1-2670 starting with group 4, second from group 5, third from group 6, forth from group 7 and then from group 8. The syringe was changed between groups. The vaccination with the inactivated HN50 batch number M-1-2671 started with group 9, following group 10, group 11, group 12 and then group 13. The syringe was changed prior to vaccination of group 9 with HN50.

Tests Methods—Efficacy Evaluation

a) Serology: At the vaccination day (antibody level at zero time) and after 21 days from vaccination, blood samples were collected from each chicken. Further, the sera were separated according to E-QCM-192 and the antibody levels were measured by using HI test according to the BSH-QCM-0000626 (E-QCM-169-03), with VH antigen and the rNDV-LS-HN G7 antigen by the European Pharmacopoeia (EP), Edition 10.5, monograph (number 07/2020:0870, subtitle 2-5-2-3, Haemagglutination inhibition).

The F50 groups were tested in NDV-F ELISA Kits (Biocheck) to have the specific F protein antibodies levels.

b) Challenge: This procedure was performed at Veterinary Services by injecting of 106.0 ELD50/dose of vvNDV gVII via IM route. After challenge, the birds were daily observed for 21 days to detect any morbidity, mortality, and reduction excretion (shedding) vNDV genotype VII virus.

c) Virus Excretion: on days 4, 6, 8 post challenge cloacal and tracheal swabs were collected from groups 1-5, 9-10. Swabs were sent to KVI for the determination of Viral load using quantitative real time PCR based on KVI method.

### Statistical Analyses

The PD50 is determined by the dose of a vaccine that may be expected to protect 50% of the chickens against the challenge dose of NDV. The PD50 also reflects the correlation between the antigen quantity, serological response, and protection from challenge.

By using the European Pharmacopoeia standards 10.2, monograph 0870, paragraph 2-3-2 as a guide regarding vaccine (Inactivated) Immunogenicity for Newcastle Disease vaccine, the vaccine should comply with the PD50 test (50% protective dose) and should not be less than 50 percent of the recommended dose, while the lower confidence limit is 35 PD50 per dose. This means that the NDV vaccine must contain a minimum of 50 PD50 in one recommended dose, and the calculation is performed by using the Reed and Muench method as presented below:

$$Proportionate distance ( P D ) = \frac{\% of survivors next above 50 \% - 50 \%}{\begin{matrix}\% of survivors next above 50 \% - \\ \% of survivors next lower 50 \%\end{matrix}}$$

The log of the 50% endpoint=dilution above 50%−(PD*log dilution factor) PD50=10−x, where x is the log of the 50% endpoint. The statistical difference between each group based on the PD50 results were determined by using the Two Way Anova model measurements design with JPM 12.0.1 software.

Assessments and Evaluable Parameters

Both tested inactivated F50 and HN50 vaccines (batches number M-1-2670 and M-1-2671) were compared in terms of protection efficacy through challenge (vNDV genotype VII) and HI test.

Further, the PD50 were calculated to evaluate of the chickens that survive in each vaccinated group without showing any signs of Newcastle Disease during a period of the 21 days post challenge.

Also, through this calculation, to verify if the vaccines comply with the test if the smallest dose stated in a dose of 0.5 mL corresponds to not less than 50 PD50.

### Disease Management

In this study care have been taken to avoid contamination between the experimental group and the negative control. When visiting the study animals, either to perform study procedures or animal husbandry, all study personnel were always attended to unvaccinated control group, then the vaccinated group. The following precautions were also taken.

Disposal of study animals: Disposal of dead birds during or at the end of the trial was by the procedures outlined into AH-016. Briefly, biological hazard waste including carcasses and excess materials were into 2 bags and should be evacuated only to the biological hazard waste container bin in the yard. The carcasses that cannot be evacuated on the same day by the authorized company should be kept in the dedicated freezer until the evacuation day.

Disposal of study products: Any excess test materials was into 2 bags and were evacuated only to biological hazard waste container.

Clothing: Gowning rules are under AH-017. According to this SOP in each entry to the inactivated vaccine department, each staff member will take off his personal clothes and were on overall and shoes belonging only to this department.

Protectives measures: The procedures used in this study are considered to be of low risk to the operators, except in case of accidental human injection with oil emulsion products, which can produce local reaction leading to severe injury (necrosis). In this situation, immediate medical attention, and treatment for removing the oil emulsified product in the affected area, should be administered to the injured personnel. For safety use, caution while injected the vaccine by proper injection technique and bird handling can reduce the potential possibility of accidents.

Postmortem: In case of post-mortem, sampling took place in the washing room, each staff member had to wear another hazmat and shoe cover according to AH-017. Once the procedure was finished, the hazmat and shoe covers belonging to the washing room were disposed as a biohazard waste according to AH-016.

### Results

HI Titers of Genotype 7 (rLaSota-HN-G7) Antigen after One Dose

All birds' sera after one dose were tested with rLasota-HN-G7 antigen. In this test, only the full dose tested by 24 serial dilutions was included to have the maximal HI potential. The diluted doses were tested by 12 serial dilutions.

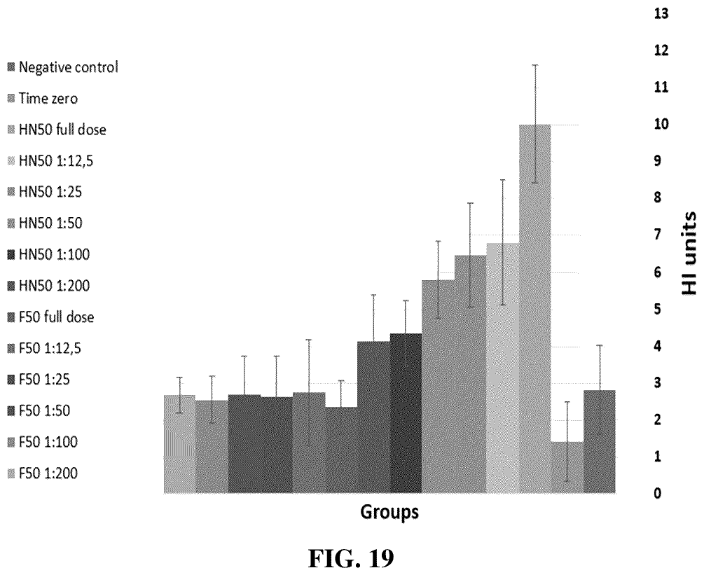

The F50 groups gave no significant readings by HI test as expected. The zero-time bleeding gave average HI score of 1.13 and the negative unvaccinated control gave low HI average score of 2.8 as expected. However, the HN50 full dose (50 μg) gave similar average score as described previously after the first vaccination. Then the diluted doses have demonstrated decreasing HI average scores that correlated with the dose dilution. The STDEV on HN50 groups decreased as the doses were diluted. These results are shown in Table 19. The HI titers against genotype 7 (rLaSota-HN-G7) antigen after one dose of treatment are shown in FIG. 19.

TABLE 19
HI Titers of Genotype 7 (rLaSota-HN-G7) Antigen After One Dose

| | HI | STANDARD | |
|---|---|---|---|
| GROUP | AVERAGE | DEV | % |
| Negative control (Unvaccinated) | 2.80 | 1.207 | 7.0 |
| F50 Positive control (full dose) | 2.33 | 0.724 | 0.0 |
| HN50 Positive control (full dose) | 10.0 | 1.604 | 100 |
| F50 dilution dose (1:12.5) | 2.73 | 1.438 | 13 |
| F50 dilution dose (1:25) | 2.60 | 1.121 | 0.0 |
| F50 dilution dose (1:50) | 2.00 | 1.047 | 7.0 |
| F50 dilution dose (1:100) | 2.53 | 0.640 | 0.0 |
| F50 dilution dose (1:200) | 2.67 | 0.488 | 0.0 |
| HN50 dilution dose (1:12.5) | 6.80 | 1.699 | 87 |
| HN50 dilution dose (1:25) | 6.47 | 1.407 | 80 |
| HN50 dilution dose (1:50) | 5.79 | 1.051 | 71 |
| HN50 dilution dose (1:100) | 4.33 | 0.900 | 13 |
| HN50 dilution dose (1:200) | 4.13 | 1.246 | 13 |
| Time 0 | 1.33 | 1.118 | 0 |

HI Titers Against Genotype 2 (VH) Antigen after One Dose

All birds' sera after one dose were tested with VH antigen. In this test, only the full dose was tested by 24 serial dilutions to have the maximal HI potential. The diluted doses were tested by 12 serial dilutions.

The F50 groups gave no significant readings by HI test as expected. The zero-time bleeding gave an average HI score of 0 and the negative unvaccinated control gave a low HI average score of 0.8. However, the HN50 full dose (50 μg) gave a lower average score as described previously after the first vaccination. The diluted doses demonstrated decreasing HI average scores that correlated with the dose dilution. The STDEV on HN50 groups were decreased as the doses were diluted.

All birds' sera form the first bleeding were tested with VH antigen. In this test, 24 serial dilutions were included to have the maximal HI potential.

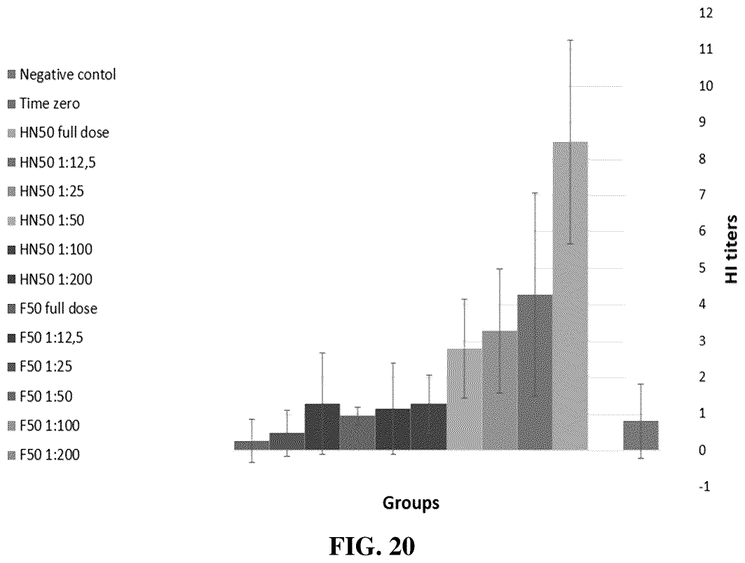

The protection levels after velogenic ND virus Genotype VII (358/2015 IVS) after one dose are shown in FIG. 20. All birds were challenged with velogenic ND virus Genotype VII (358/2015 IVS). The test was valid. All HN50 groups have shown high protection.

TABLE 20
Mortality Table by The End of The PD50 Trial

| GROUP | DEAD/TOTAL | PROTECTION (%) |
|---|---|---|
| Non-vaccinated | 15/15 | 0 |
| F50 full dose | 15/15 | 0 |
| HN50 full dose | 15/0 | 100 |
| F50 1:12.5 | 15/15 | 0 |
| F50 1:25 | 14/15 | 7 |
| F50 1:50 | 15/15 | 0 |
| F50 1:100 | 15/15 | 0 |
| F50 1:200 | 15/15 | 0 |
| HN50 1:12.5 | 1/15 | 93 |
| HN50 1:25 | 1/15 | 93 |
| HN50 1:50 | 1/14 | 93 |
| HN50 1:100 | 1/15 | 93 |
| HN50 1:200 | 15/4 | 73 |

HN50 Antigen PD50 Determination

By using the World Organization (OIE) for Animal Health Newcastle Disease vaccine standards as a guide, the vaccine complies with the test if the PD50 (50% protective dose) is not less than 50 per recommended dose and if the lower confident limit is 35 PD50 per dose. The calculation is performed using the Reed and Muench method as exemplified bellow:

$$Proportionate distance ( P D ) = \frac{\% of survivors next above 50 \% - 50 \%}{\begin{matrix}\% of survivors next above 50 \% - \\ \% of survivors next lower 50 \%\end{matrix}}$$

The log of the 50% endpoint=dilution above 50%−(PD*log dilution factor) PD50=10−x, where x is the log of the 50% endpoint

TABLE 21
Version A-Scenario Where Treatment Group 1:400 Does Not Survive

| | | | | | | | | Percentage |
|---|---|---|---|---|---|---|---|---|
| Dose | | Vol | | | Total | Total | Survivors + | of |
| (dilution) | Log | (μL) | Survivors | Death | survivals | death | death | protection |
| 1:12.5 | 10-1.1 | 40 | 14 | 1 | 66 | 1 | 67 | 98.5 |
| 1:25 | 10-1.4 | 20 | 14 | 1 | 52 | 2 | 54 | 96.3 |
| 1:50 | 10-1.7 | 10 | 13 | 1 | 38 | 3 | 41 | 92.7 |
| 1:100 | 10-2 | 5 | 14 | 1 | 25 | 4 | 29 | 86.2 |
| 1:200 | 10-2.3 | 2.5 | 11 | 4 | 11 | 8 | 19 | 57.9 |
| 1:400* | 10-2.6 | 1.25 | 0 | 15 | 0 | 23 | 23 | 0 |

*Theorical assumption for calculation purpose.
100% (15 from 15) chickens die, 0% of chickens survive.
It is number next lower 50%

$$P D 50 = \frac{57.9 - 50}{57.9 - 0} = \frac{7.9}{57.9} = 0.136$$

Log of the 50% endpoint=−2.6−(0.136*0.3)=10 2.64=436.5 PD50/dose.

TABLE 22
Version B-Scenario Where 50% of Treatment Group 1:400 Does Not Survive

| | | | | | | | | PERCENTAGE |
|---|---|---|---|---|---|---|---|---|
| DOSE | | VOL | | | TOTAL | TOTAL | SURVIVORS + | OF |
| (DILUTION) | LOG | (μL) | SURVIVORS | DEATH | SURVIVALS | DEATH | DEATH | PROTECTION |
| 1:12,5 | 10-1.1 | 40 | 14 | 1 | 73 | 1 | 74 | 98.6 |
| 1:25 | 10-1.4 | 20 | 14 | 1 | 59 | 2 | 61 | 96.7 |
| 1:50 | 10-1.7 | 10 | 13 | 1 | 45 | 3 | 48 | 93.7 |
| 1:100 | 10-2 | 5 | 14 | 1 | 32 | 4 | 36 | 88.9 |
| 1:200 | 10-2.3 | 2.5 | 11 | 4 | 18 | 8 | 26 | 69.2 |
| 1:400* | 10-2.6 | 1.25 | 7 | 8 | 7 | 16 | 23 | 30.4 |

*Theorical assumption for calculation purpose.
53% (7 from 15) chickens die, 47% chickens survive.
It is number next lower 50%.

$$P D 50 = \frac{69.2 - 50}{69.2 - 30.4} = \frac{19.2}{38.8} = 0.49$$

Log of the 50% endpoint=−2.6−(0.49*0.3)=−2.6−(0.147)=10 2.747=558 PD50/dose

ELISA-NDV-F Titers of F50 Group after One Dose

F50 birds' sera form both bleedings were tested on NDV-F ELISA. The test was valid.

TABLE 23
NDV-F ELISA Averages Titers With STDEV

| GROUP | ANTIBODY TITER | STDEV |
|---|---|---|
| Not vaccinated | 218 | 318 |
| F50 full dose | 13,476 | 7,560 |
| F50 1:12.5 | 3,250 | 2,651 |
| F50 1:25 | 1,607 | 1,550 |
| F50 1:50 | 1,137 | 975 |
| F50 1:100 | 518 | 520 |

To explain the lack of protection of F50 full dose group a comparison between previous studies to the current study was conducted (Table 23). F50 full dose in the current study gave average titers of 13,476 with STDEV of 7,560. All birds in this group died after challenge. F50 full dose in previous studies gave average titers of 10,116 with STDEV of 6,128. No challenge was done after the first dose. The challenge was done 21 after the boost vaccination. The ELISA NDV-F titers after one dose of F50 were similar between both studies.

NECTIV FORTE after the prime dose in previous studies gave average titers of 27,297 with STDEV of 4,480. No challenge was done after the first dose. The challenge was done 21 after the boost vaccination. After the NECTIV FORTE boost vaccination the titers were 41,052 with STDEV of 2,556. This group gave 90% protection after challenge.

In previous studies after the F50 boost vaccination, the titers were 32,853 with STDEV of 11,967. This group gave 90% protection after challenge.

The conclusion from this comparison is that one dose of F50 was not enough to induce high antibodies levels and thus the protection level was suboptimal.

NDV Secretion from HN50 Vaccinated and Challenged Chicken

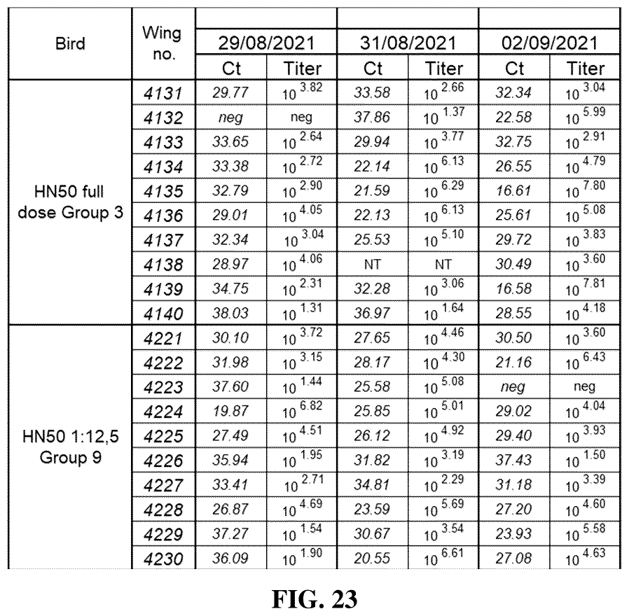

4, 6 and 8 days after challenge tracheal and cloacal swabs were collected from group 3 (HN50 full dose) and from group 9 (HN50 1:12.5) and were submitted for viral RNA quantification using qPCR method (KVI method). Results are shown in FIG. 23.

TABLE 24
ELISA NDV-F Raw Data

| | | | | | | | | TITER |
|---|---|---|---|---|---|---|---|---|
| | WING | | READING | | LOG10 | | TITER | STDE |
| GROUP | NO. | READING | AV | S/P | TITER | TITER | AV | V |
| NC | NC | 0.106 | | | | | | |
| NC | NC | 0.105 | 0.106 | | | | | |
| PC | PC | 0.34 | | | | | | |
| PC | PC | 0.36 | 0.350 | | | | | |
| Un- | 4101 | 0.107 | | 0.004 | 1.133 | 14 | | |
| vaccinated | | | | | | | | |
| Un- | 4102 | 0.108 | | 0.008 | 1.434 | 27 | | |
| vaccinated | | | | | | | | |
| Un- | 4103 | 0.112 | | 0.025 | 1.911 | 81 | | |
| vaccinated | | | | | | | | |
| Un- | 4104 | 0.129 | | 0.094 | 2.494 | 312 | | |
| vaccinated | | | | | | | | |
| Un- | 4105 | 0.11 | | 0.016 | 1.735 | 54 | | |
| vaccinated | | | | | | | | |
| Un- | 4106 | 0.116 | | 0.041 | 2.133 | 136 | | |
| vaccinated | | | | | | | | |
| Un- | 4107 | 0.212 | | 0.434 | 3.158 | 1439 | | |
| vaccinated | | | | | | | | |
| Un- | 4108 | 0.138 | | 0.131 | 2.638 | 434 | | |
| vaccinated | | | | | | | | |
| Un- | 4109 | 0.13 | | 0.098 | 2.513 | 326 | | |
| vaccinated | | | | | | | | |
| Un- | 4110 | 0.111 | | 0.020 | 1.832 | 68 | | |
| vaccinated | | | | | | | | |
| Un- | 4111 | 0.113 | | 0.029 | 1.978 | 95 | | |
| vaccinated | | | | | | | | |
| Un- | 4112 | 0.118 | | 0.049 | 2.212 | 163 | | |
| vaccinated | | | | | | | | |
| Un- | 4113 | 0.109 | | 0.012 | 1.610 | 41 | | |
| vaccinated | | | | | | | | |
| Un- | 4114 | 0.109 | | 0.012 | 1.610 | 41 | | |
| vaccinated | | | | | | | | |
| Un- | 4115 | 0.109 | 0.122 | 0.012 | 1.610 | 41 | 218 | 361 |
| vaccinated | | | | | | | | |
| F50 | 4116 | 0.563 | | 1.873 | 3.793 | 6202 | | |
| full dose | | | | | | | | |
| F50 | 4117 | 1.087 | | 4.020 | 4.124 | 13313 | | |
| full dose | | | | | | | | |
| F50 | 4118 | 0.537 | | 1.766 | 3.767 | 5849 | | |
| full dose | | | | | | | | |
| F50 | 4119 | 0.491 | | 1.578 | 3.718 | 5225 | | |
| full dose | | | | | | | | |
| F50 | 4120 | 0.744 | | 2.615 | 3.937 | 8658 | | |
| full dose | | | | | | | | |
| F50 | 4121 | 0.755 | | 2.660 | 3.945 | 8808 | | |
| full dose | | | | | | | | |
| F50 | 4122 | 1.409 | | 5.340 | 4.248 | 17683 | | |
| full dose | | | | | | | | |
| F50 | 4123 | 1.325 | | 4.996 | 4.219 | 16543 | | |
| full dose | | | | | | | | |
| F50 | 4124 | 2.356 | | 9.221 | 4.485 | 30535 | | |
| full dose | | | | | | | | |
| F50 | 4125 | 0.84 | | 3.008 | 3.998 | 9961 | | |
| full dose | | | | | | | | |
| F50 | 4126 | 1.761 | | 6.783 | 4.351 | 22460 | | |
| full dose | | | | | | | | |
| F50 | 4127 | 1.111 | | 4.119 | 4.135 | 13639 | | |
| full dose | | | | | | | | |
| F50 | 4128 | 0.888 | | 3.205 | 4.026 | 10612 | | |
| full dose | | | | | | | | |
| F50 | 4129 | 1.926 | | 7.459 | 4.393 | 24699 | | |
| full dose | | | | | | | | |
| F50 | 4130 | 0.692 | 1.099 | 2.402 | 3.901 | 7953 | 13476 | 7560 |
| full dose | | | | | | | | |
| F50 | 4146 | 0.295 | | 0.775 | 3.409 | 2565 | | |
| 1:12,5 | | | | | | | | |
| F50 | 4147 | 0.178 | | 0.295 | 2.990 | 977 | | |
| 1:12,5 | | | | | | | | |
| F50 | 4148 | 0.136 | | 0.123 | 2.610 | 407 | | |
| 1:12,5 | | | | | | | | |
| F50 | 4149 | 0.153 | | 0.193 | 2.805 | 638 | | |
| 1:12,5 | | | | | | | | |
| F50 | 4150 | 0.632 | | 2.156 | 3.854 | 7138 | | |
| 1:12,5 | | | | | | | | |
| F50 | 4151 | 0.156 | | 0.205 | 2.832 | 679 | | |
| 1:12,5 | | | | | | | | |
| F50 | 4152 | 0.264 | | 0.648 | 3.331 | 2144 | | |
| 1:12,5 | | | | | | | | |
| F50 | 4153 | 0.504 | | 1.631 | 3.732 | 5401 | | |
| 1:12,5 | | | | | | | | |
| F50 | 4154 | 0.689 | | 2.389 | 3.898 | 7912 | | |
| 1:12,5 | | | | | | | | |
| F50 | 4155 | 0.523 | | 1.709 | 3.753 | 5659 | | |
| 1:12,5 | | | | | | | | |
| F50 | 4156 | 0.199 | | 0.381 | 3.101 | 1262 | | |
| 1:12,5 | | | | | | | | |
| F50 | 4157 | 0.159 | | 0.217 | 2.857 | 719 | | |
| 1:12,5 | | | | | | | | |
| F50 | 4158 | 0.271 | | 0.676 | 3.350 | 2239 | | |
| 1:12,5 | | | | | | | | |
| F50 | 4159 | 0.569 | | 1.898 | 3.798 | 6283 | | |
| 1:12,5 | | | | | | | | |
| F50 | 4160 | 0.454 | 0.345 | 1.426 | 3.674 | 4723 | 3250 | 2651 |
| 1:12,5 | | | | | | | | |
| F50 | 4161 | 0.469 | | 1.488 | 3.693 | 4926 | | |
| 1:25 | | | | | | | | |
| F50 | 4162 | 0.466 | | 1.475 | 3.689 | 4886 | | |
| 1:25 | | | | | | | | |
| F50 | 4163 | 0.195 | | 0.365 | 3.082 | 1208 | | |
| 1:25 | | | | | | | | |
| F50 | 4164 | 0.349 | | 0.996 | 3.518 | 3298 | | |
| 1:25 | | | | | | | | |
| F50 | 4165 | 0.126 | | 0.082 | 2.434 | 271 | | |
| 1:25 | | | | | | | | |
| F50 | 4166 | 0.225 | | 0.488 | 3.208 | 1615 | | |
| 1:25 | | | | | | | | |
| F50 | 4167 | 0.141 | | 0.143 | 2.677 | 475 | | |
| 1:25 | | | | | | | | |
| F50 | 4168 | 0.207 | | 0.414 | 3.137 | 1371 | | |
| 1:25 | | | | | | | | |
| F50 | 4169 | 0.117 | | 0.045 | 2.174 | 149 | | |
| 1:25 | | | | | | | | |
| F50 | 4170 | 0.226 | | 0.492 | 3.212 | 1629 | | |
| 1:25 | | | | | | | | |
| F50 | 4171 | 0.159 | | 0.217 | 2.857 | 719 | | |
| 1:25 | | | | | | | | |
| F50 | 4172 | 0.223 | | 0.480 | 3.201 | 1588 | | |
| 1:25 | | | | | | | | |
| F50 | 4173 | 0.168 | | 0.254 | 2.925 | 841 | | |
| 1:25 | | | | | | | | |
| F50 | 4174 | 0.157 | | 0.209 | 2.840 | 692 | | |
| 1:25 | | | | | | | | |
| F50 | 4175 | 0.138 | 0.224 | 0.131 | 2.638 | 434 | 1607 | 1550 |
| 1:25 | | | | | | | | |
| F50 | 4176 | 0.384 | | 1.139 | 3.577 | 3773 | | |
| 1:50 | | | | | | | | |
| F50 | 4177 | 0.163 | | 0.234 | 2.888 | 774 | | |
| 1:50 | | | | | | | | |
| F50 | 4178 | 0.2 | | 0.385 | 3.106 | 1276 | | |
| 1:50 | | | | | | | | |
| F50 | 4179 | 0.163 | | 0.234 | 2.888 | 774 | | |
| 1:50 | | | | | | | | |
| F50 | 4180 | 0.133 | | 0.111 | 2.564 | 366 | | |
| 1:50 | | | | | | | | |
| F50 | 4181 | 0.245 | | 0.570 | 3.276 | 1886 | | |
| 1:50 | | | | | | | | |
| F50 | 4182 | 0.295 | | 0.775 | 3.409 | 2565 | | |
| 1:50 | | | | | | | | |
| F50 | 4183 | 0.203 | | 0.398 | 3.119 | 1316 | | |
| 1:50 | | | | | | | | |
| F50 | 4184 | 0.199 | | 0.381 | 3.101 | 1262 | | |
| 1:50 | | | | | | | | |
| F50 | 4185 | 0.186 | | 0.328 | 3.036 | 1086 | | |
| 1:50 | | | | | | | | |
| F50 | 4186 | 0.146 | | 0.164 | 2.735 | 543 | | |
| 1:50 | | | | | | | | |
| F50 | 4187 | 0.12 | | 0.057 | 2.279 | 190 | | |
| 1:50 | | | | | | | | |
| F50 | 4188 | 0.129 | | 0.094 | 2.494 | 312 | | |
| 1:50 | | | | | | | | |
| F50 | 4189 | 0.148 | | 0.172 | 2.756 | 570 | | |
| 1:50 | | | | | | | | |
| F50 | 4190 | 0.133 | 0.190 | 0.111 | 2.564 | 366 | 1137 | 975 |
| 1:50 | | | | | | | | |
| F50 | 4191 | 0.117 | | 0.045 | 2.174 | 149 | | |
| 1:100 | | | | | | | | |
| F50 | 4192 | 0.124 | | 0.074 | 2.388 | 244 | | |
| 1:100 | | | | | | | | |
| F50 | 4193 | 0.123 | | 0.070 | 2.363 | 231 | | |
| 1:100 | | | | | | | | |
| F50 | 4194 | 0.158 | | 0.213 | 2.849 | 706 | | |
| 1:100 | | | | | | | | |
| F50 | 4195 | 0.237 | | 0.537 | 3.250 | 1778 | | |
| 1:100 | | | | | | | | |
| F50 | 4196 | 0.121 | | 0.061 | 2.309 | 204 | | |
| 1:100 | | | | | | | | |
| F50 | 4197 | 0.112 | | 0.025 | 1.911 | 81 | | |
| 1:100 | | | | | | | | |
| F50 | 4198 | 0.138 | | 0.131 | 2.638 | 434 | | |
| 1:100 | | | | | | | | |
| F50 | 4199 | 0.118 | | 0.049 | 2.212 | 163 | | |
| 1:100 | | | | | | | | |
| F50 | 4200 | 0.134 | | 0.115 | 2.580 | 380 | | |
| 1:100 | | | | | | | | |
| F50 | 4201 | 0.126 | | 0.082 | 2.434 | 271 | | |
| 1:100 | | | | | | | | |
| F50 | 4202 | 0.145 | | 0.160 | 2.724 | 529 | | |
| 1:100 | | | | | | | | |
| F50 | 4203 | 0.113 | | 0.029 | 1.978 | 95 | | |
| 1:100 | | | | | | | | |
| F50 | 4204 | 0.18 | | 0.303 | 3.002 | 1004 | | |
| 1:100 | | | | | | | | |
| F50 | 4205 | 0.216 | 0.144 | 0.451 | 3.174 | 1493 | 518 | 520 |
| 1:100 | | | | | | | | |

### Conclusions

HN50 antigen generated high levels of antibodies against both genotype 7 and 2 strains with high percentage of protection. The PD50 of HN50 was calculated in both ways and it is in the range of 436.5 to 558 PD50 per dose.

In addition, the recombinant F50 antigen did not generate high levels of antibodies against the challenge with a velogenic ND virus Genotype VII (358/2015 IVS). In summary, a proof-of-concept demonstration was accomplished for HN50 antigen by this trial.

TABLE 25
HI Scores (Serology) Before Challenge and Mortality Scores After Challenge

| | LASOTA HN - | VH STRAIN | |
|---|---|---|---|
| | GENOTYPE 7 | GENOTYPE 2 | |
| GROUP | HI AVERAGE | HI AVERAGE | DEAD/TOTAL |
| Negative control (Unvaccinated) | 2.80 | 0.80 | 15/15 |
| F50 Positive control (full dose) | 2.33 | 0.93 | 15/15 |
| HN50 Positive control (full dose) | 10.0 | 8.47 | 15/0 |
| F50 dilution dose (1:12.5) | 2.73 | 1.27 | 15/15 |
| F50 dilution dose (1:25) | 2.60 | 0.47 | 14/15 |
| F50 dilution dose (1:50) | 2.00 | 0.27 | 15/15 |
| F50 dilution dose (1:100) | 2.53 | 0.00 | 15/15 |
| F50 dilution dose (1:200) | 2.67 | 0.00 | 15/15 |
| HN50 dilution dose (1:12.5) | 6.80 | 4.27 | 1/15 |
| HN50 dilution dose (1:25) | 6.47 | 3.27 | 1/15 |
| HN50 dilution dose (1:50) | 5.79 | 2.79 | 1/14 |
| HN50 dilution dose (1:100) | 4.33 | 1.27 | 1/15 |
| HN50 dilution dose (1:200) | 4.13 | 1.13 | 4/15 |
| Time 0 | 1.33 | 0.00 | ND |

*ND = Not done, The HI values are an average of 15 birds' group each

### Example 4

### Expression of NDV Antigens in C1 System

In this example, two additional strains were constructed. A first strain constructed comprised four copies of HN50 expression cassette, two copies cbhI and two copies of bgl8 locus. A second strain constructed comprised with two copies of expression cassette in bgl8 locus for F50 protein with T4 fibritin trimerization domain. The NDV antigen production levels for target protein NDV F50-Ctag for a single copy of expression cassette was 0.3 g/l and for the two copies of expression cassettes was 0.3 g/1. The NDV production levels for NDV HN50-Ctag for the single copy of expression cassette was 1.1 g/l and for the two copies of expression cassettes was 4.2 g/1.

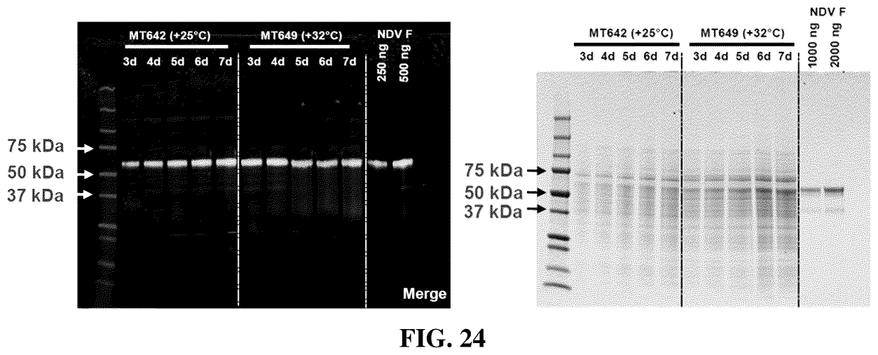

FIG. 24 provides images of gels used to analyze additional outputs from bioreactor fermentation runs generating the NDV F50-protein producing strains. MT642 run was performed with NDV F50-producing C1 strain M6316, which is a clone purified from transformant M5824 (chitinase-deletion strain/2×NDV F50-producing strain with the addition of trimerization domain) at 25° C. The MT649 was performed with alternative NDV F50-protein producing C1 strain M6318, which is a clone purified from M5824 transformant (chitinase-deletion strain/2×NDV F50-producing strain with the addition of trimerization domain) at 32° C. The fermentation supernatant had a pinkish coloration and 0.5 μL was loaded per well. Target protein detection was performed with NDV antiserum/rabbit anti-chicken Ig-IRD680 (shown in red) and biotin-α-Ctag/Streptavidin-IRD800 (shown in green). An improved production level of the NDV F50-producing strain was achieved in the additional run, with the alternative NDV F50-producing strain at 32° C. producing 1.7 g/L after six days, and 1.9 g/L after 7 days.

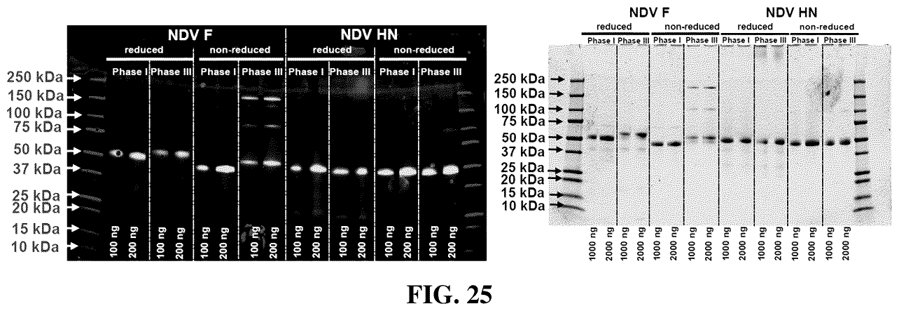

FIG. 25 provides images of gels analyzing purified C1 NDV F and HN antigens expressed using a C1 system on Western blot and stained gel under reducing and non-reducing conditions. Target protein detection was performed using NDV anti-serum/rabbit anti-chicken IgIRD 680 (shown in red) and biotin-α-Ctag/Streptavidin-IRD800 (shown in green). A merge of the images (yellow) indicates where the signals overlap. Purified proteins showed good stability and the addition of the trimerization domain to the NDV F antigen resulted in a molecular weight increase by approximately kDa. Moreover, putative oligomers (dimers and trimers) can be seen in non-reduced samples of Phase III NDV F Antigen. NDV HN antigens produced in Phase I and Phase III show identical gel mobility. Production of NDV F antigen with trimerization domain preliminary estimated at about 1.9 g/L was achieved and NDV HN50 antigen with 4 copies of HN50 expression cassette was estimated to provide a production level of 10 g/L. Summary of the strains used in this Example are provided in Table 26.

TABLE 26
Summary of Strains for Example 4

| | | | | | Production | |
|---|---|---|---|---|---|---|
| | Host | | | Copy | Level at 1 | |
| Target Protein | Strain | Promoter | Locus | Number | L Scale | |
| NDV F50-Ctag | M5824 (14× | bgl8pr | bgl8 | 2× | 1.9 | g/L |
| with fibritin | protease Δ; | | | | | |
| trimerization | Δchil.pyr4-) | | | | | |
| domain | | | | | | |
| NDV HN50- | M5824 (15× | bgl8pr | cbh1 + | 4× | 10 | g/L |
| Ctag | protease Δ; | | bgl8 | | | |
| | Δchil.pyr4-) | | | | | |

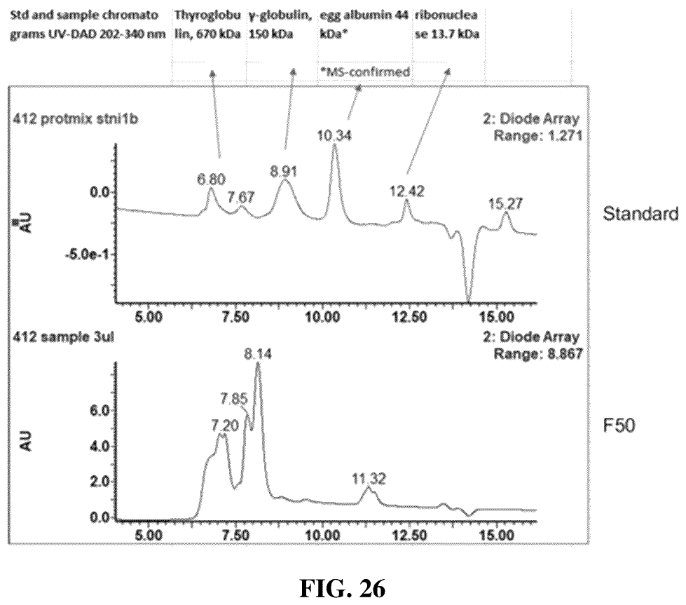

SEC-UV: A standard protein mix and sample comprising NDV-F50 protein with the trimerization domain, with an expected monomer mass with oxidized disulfide bridges (10 cysteines) of 53,376.3 Da (average mass by NIST protein calculator), was analyzed by UV-DAD chromatography with PBS buffer: 12 mM NaH₂PO₄*H₂O, 150 mM NaCl pH 7.3. FIG. 26 shows the SEC-UV in PBS buffer chromatograms (202-340 nm) illustrating that the standard sample was highly similar to manufacturer's chromatogram and the F50 sample showed broad early peaks and two sharper peals eluting at 7.85 min and 8.14 min. The calculated mass of the monomer is 53,376.3 Da; therefore, the trimers would be 160.1 kDa. Predicted mass of main peak based on SEC-UV is approximately 250 kDa. The early eluting broad peaks are likely due to aggregation and/or oligomerization.

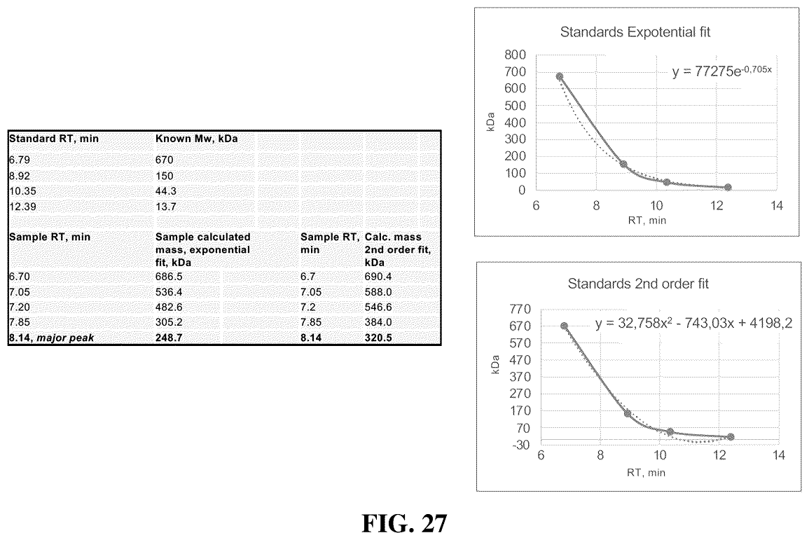

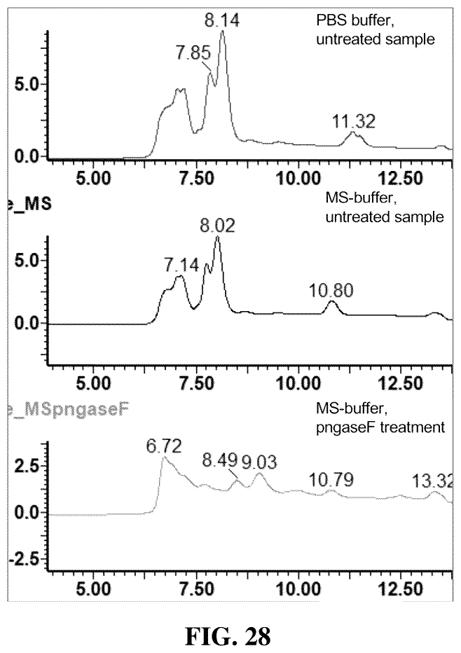

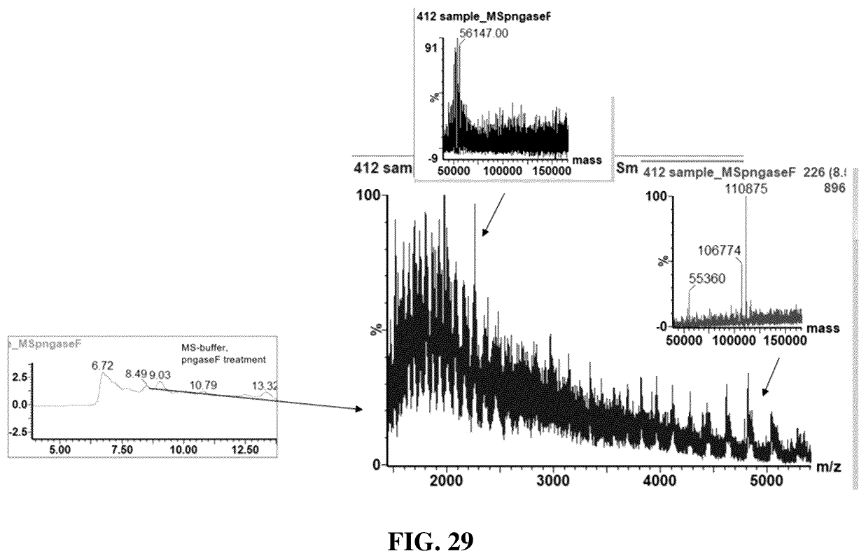

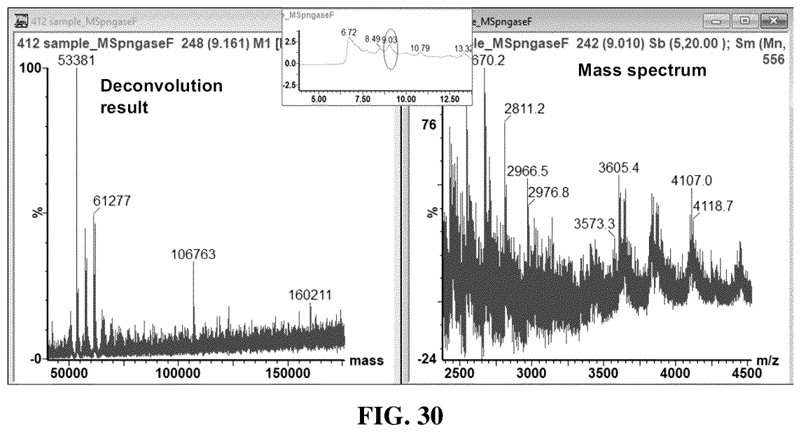

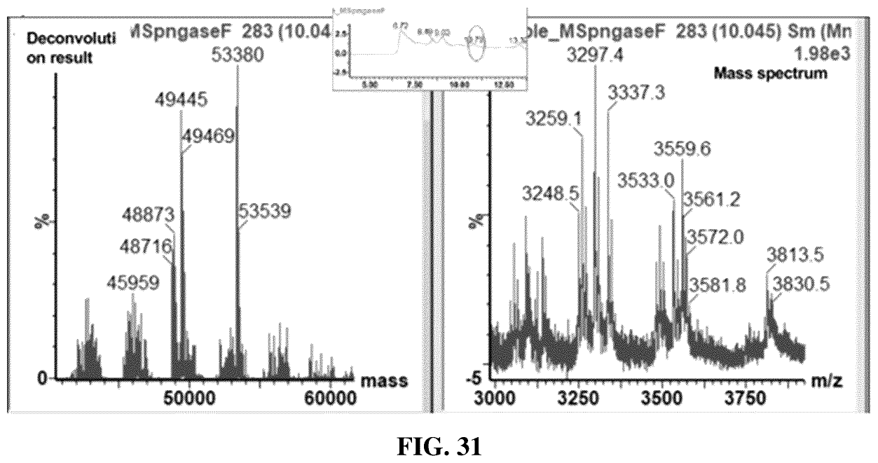

SEC-UV-MS: Analyzed by UV-DAD chromatography with 40 mM ammonium acetate, unbuffered, and a pH of approximately 6 compared to untreated sample in PBS buffer, and to untreated sample in MS-buffer. FIG. 28 shows the SEC-UV-chromatograms of untreated sample (PBS buffer), untreated sample (MS-Buffer), and MS-buffer PngaseF treatment, which was done using minimally denaturing conditions (Rapid PngaseF buffer 1:5 dilution, Rapid PngaseF enzyme approximately at 1:30 protein ratio for four hours at 40° C.). FIG. 28 shows the combined spectra from chromatogram of untreated samples produces unresolved protein spectrum at all time points, and the PngaseF treated sample gave a spectrum from several time points. Deconvolution of m/z region around 2000 shows proteins around 50 kDa to 58 kDa. Deconvolution of m/z region around 4500 shows proteins around of 106 and 110 kDa. In view of FIG. 29, the spectrum from 8.5 minutes shows the m/z dependent existence of monomeric and dimeric approximately 55 kDa and 11 kDa protein, and the SEC-UV exponential fit for 8.5 min predicts 180 kDa mass (see FIG. 27). FIG. 30 is the MS spectrum of PNGase-treated sample showing the spectra combined and deconvoluted from main peak at 9.1 minutes. Moreover, the dimeric and monomeric are seen, with some evidence of the trimeric protein. The monomeric deconvolution result at 53,381 Da is close to the theoretical mass. FIG. 31 shows the MS spectra of PNGase-treated sample combined from peak at 10 minutes (SEC predicted mass approximately 50 kDa. Deconvolution shows masses at 49,445 Da and at 53,880 Da, the latter close to the theoretical mass. The 3.7 Da difference from theoretical could be attributed to the open disulfide pair and deamidation.

The SEC-UV-MS results with MS compatible buffer show similar UV elution to PBS buffer. MS spectra show that without PngaseF treatment, MS spectrum does not resolve enough for deconvolution (MW estimation). The PngaseF treatment reveals dimeric and monomeric masses eluting at three to four different time points because complexes likely broke up in the ionization source and/or because mild denaturant was used with PngaseF, creating partly disturbed complexes.

The monomeric protein mass is very close to theoretical mass, and thus the protein expression was successful.

### Example 5

### NDV Dose Response for HN50 and F50 Antigens from C1 Expression System in SPF Chickens

### Background

As previously discussed, Newcastle disease (ND) is a highly contagious viral disease that can affect all bird species. This enveloped virus (Avulavirus genus, Paramyoxoviridae family, Mononegavirales order, recently renamed as Avian Avulavirus or AAvV) has a marked genomic variability and the genotypes V, VI, VII and VIII (from class II) are predominant in different countries all over the world.

The clinical signs related to the infection are described as respiratory, gastrointestinal and neurologic signs which depend on the animal immune status and susceptibility and the strain involved. Depending on their pathogenicity, NDV viruses can be also classifies as Velogenic Viscerotropic (ability to cause necro-haemorrhagic lesions in the intestine, cause up to 100% morbidity and mortality in naive chickens, vvNDV); Mesogenic (respiratory and neurological signs with moderate morbidity and mortality); and Lentogenic (minimal respiratory disease observed, especially when associated with secondary bacterial infection, used as live vaccines).

Inactivated ND vaccine (Nectiv Forte™) is used routinely to vaccinate chickens and turkeys. The vaccine provides slow response but longer duration of immunity. The vaccine formulation is water-in-oil emulsion and is based on mineral oil plus Montane/Montaneox as emulsifiers. To produce the NDV antigens for inactivated ND vaccines, the virus is propagated in embryonic eggs. This vaccine is based on LaSota backbone expressing HN gene of NDV G7. The positive control in this example was the NDV G7 vaccine.

As disclosed herein, C1 is used to produce immunogenic recombinant antigens (HN or F proteins) in a fungus cell line in order to replace the ND vaccine production in embryonic eggs. C1 has a unique morphology allowing for hyper productivity and growth under low viscosity media using a broad range of fermentation conditions at flexible commercial scales. This expression system provides high-yield, low cost, rapidly scalable, rVaccine-antigens and antibody gene expression platform in single use or stainless-steel bioreactors. It offers safe and effective antigens, generally recognized as safe (GRAS) certification from US FDA, and no negative clinical effects have been observed in animal studies.

In previous studies, the minimal dose of HN protein was found in PD50 trials to be between 13.5 μg to 17 μg per dose (equal to 150PD50 dose). The purified protein process includes centrifugation at 7000 g, then the supernatant is subject to filtration using 0.45 μm filters. The filtrate could be further loaded on c-tag affinity column to obtain pure protein.

### Objectives

The objectives of this study are (1) to test the dose response of HN50 pure protein, 10 kDa ultrafiltered material after centrifugation and filtrate after 0.45 μM filtration; and (2) to test the dose response of F50 pure protein from Phase 3 in prime and boost maximal doses.

### Study Design

Treatment Groups

195 SPF chicks were hatched in Har-Tuv animal houses and separated into 12 groups. Groups 1-10 included 12 chickens each, while groups 11 and 12 included 24 chickens each. This includes eleven vaccinated groups and one non-nonvaccinated control group. At 21 days of age, blood samples were randomly taken from the chicks prior to vaccination (time zero), the chickens were neck tagged from groups 1-12, and the birds in each group were vaccinated with an appropriate formulation by intramuscular (I.M.) injection with a volume of 0.3 mL per dose/chicken according to Table 27. At 42 days old (three weeks after vaccination) the chicks in all groups were bled and the samples identified individually for further testing of the HI antibody levels against the NDV (the HI titters will be determined using the HI method QCM-169, with two antigens: VH and rNDV-LS-HN G7). Groups 1-10 will be euthanized after the first bleeding.

Twelve chickens from groups 11 and 12 were NDV challenged on day 43. The rest of the twelve chickens in group 11 were vaccinated with boost vaccination and bled on day 63 (with group 12). On day 71, the chickens were again NDV challenged. This schedule of events is summarized in Table 28.

TABLE 27
Study Groups

| | | | Conc./ | Chicken |
|---|---|---|---|---|
| Group | Description | Batch | dose | per group |
| 1 | NDV genotype 7 inactivated vaccine | A0022131005 | 0.3 mL | 12 |
| 2 | HN50 pure protein (Phase 2) | M-1-3274 | 15 μg/0.3 mL | 12 |
| 3 | HN50 pure protein (Phase 2) | M-1-3275 | 50 μg/0.3 mL | 12 |
| 4 | HN50 pure protein (Phase 2) | M-1-3276 | 100 μg/0.3 mL | 12 |
| 5 | HN50 ultrafiltrated material (Phase 2) | M-1-3277 | 15 μg/0.3 mL | 12 |
| 6 | HN50 ultrafiltrated material (Phase 2) | M-1-3278 | 50 μg/0.3 mL | 12 |
| 7 | HN50 ultrafiltrated material (Phase 2) | M-1-3279 | 75 μg/0.3 mL | 12 |
| 8 | HN50 pure protein (Phase 3) | M-1-3280 | 15 μg/0.3 mL | 12 |
| 9 | HN50 pure protein (Phase 3) | M-1-3281 | 50 μg/0.3 mL | 12 |
| 10 | HN50 pure protein (Phase 3) | M-1-3282 | 100 μg/0.3 mL | 12 |
| 11 | F50 pure protein (Phase 3) | M-1-3283 | 50 μg/0.3 mL | 24 |
| 12 | Non-vaccinated | NA | NA | 24 |

### Schedule of Events

TABLE 28
Trial Events

| Trial day | Action |
|---|---|
| 1 | Hatching of SPF chicks. |
| 21 | Neck tag. |
| | Bleeding for serology time zero - 10 chickens randomly |
| | selected from the large group (10% sampling) |
| | Vaccination for groups 1-11. |
| 21-42 | Veterinary inspection for vaccination clinical signs |
| | evaluation. (According to AH-004Form02) |
| 42 | Bleeding for serology, separation by group. |
| 43 | Euthanasia of the chickens for groups 1-10. Send 12 |
| | chickens from groups 11 and 12 to vvNDV challenge. |
| 42 | Vaccination for group 11. |
| 63 | Bleeding for serology, separation by group. |
| 71 | Send the chickens for groups 11 and 12 to vvNDV |
| | challenge and end of the trial. |

*Deviation of ±3 day were acceptable

### Materials

Test System—Animals

The animals used in this study were day-old SPF chicks, Gallus gallus spp., White Leghorn breed that were hatched from SPF eggs from a SPF flock. Both male and female chicks were used.

TABLE 29
Detailed Information of the Test System Performed

| | |
|---|---|
| Species: | Day old Specified Pathogen Free (SPF) chicks - Gallus gallus |
| | spp. White Leghorn breed will be hatched from SPF eggs from a SPF flock. |
| Supplier flock number | batch no.: SPAFAS, Flock: N112 Batch: 032932-2023 |
| Initial Age | One day old chickens at day 0 (zero) |
| Serological Status | The COA of the SPF embryonated eggs used, will be attached to the report. |
| Sex | Male or female |
| Origen | Embryonated SPF eggs will be sourced from a commercial |
| | supplier and incubated within the SPF hatchery at Phibro's Test |
| | Facility (according to WI-LV-004) until enrolment onto the |
| | study at 18 days old embryo. Then, the well-developed embryos |
| | will be transferred to the Test Facility Test Site (with filled and |
| | signed QCM-190Form01). Upon arrival to the Test Facility Test |
| | Site, the eggs will be placed into the hatchery according to AH-016. |
| Hatching | According to AH-001 and QCM-190Form01. Hatching the |
| | embryonated SPF and transfer to the inactivated room. SPF |
| | chickens will be from the same SPF flock and batch of eggs. |
| | From the beginning through the duration of the study the birds |
| | will be subjected to daily inspection of the animal care and |
| | welfare office for the daily records (according to the AH- |
| | 004Form02). Only chickens showing normal behaviour |
| | (according to AH-004Form02) will be selected for the study |

Inclusion/Exclusion/Withdrawal Criteria

To be included in this study, chicks had to satisfy the following requirements: (1) originate from the same SPF flock and batch of eggs; and (2) be healthy chicks and free of signs of disease upon enrolment as determined by normal appearance and normal behaviour.

During the study, any sick or dead birds were examined by the responsible veterinarian surgeon and the cause recorded into BSH-FRM-0004234. In case of any cause of morbidity/mortality unrelated to vaccination the birds will be excluded from the analysis of results. During the whole study period, animals withdrawing were conducted according to BSH-SOP-0000231. The number of animals withdrawn prematurely from the studies and the reasons for such withdrawal were recorded.

Animal Management

The animal management and husbandry procedures are shown in Table 30.

TABLE 30
Animal Management

| | |
|---|---|
| Animal welfare | We declare and guarantee that all animal studies for rNDV-LS- |
| | NDV-HN G7 vaccine will be in line with the relevant legislation in |
| | EU (Directive 2010/63/EU) and Israel and in accordance with Israeli |
| | animal welfare legislation. |
| Housing | Study animals will be mixed and allocated in cages in the inactivated |
| | room, the whole period of study, in Phibro's Test Facility - test site |
| | at Har Tuv. |
| Food and water | Tap water and food will be available ad libitum. Food will be in the |
| | form of standard rations appropriate to the age of the birds: Standard |
| | chick diet (Tadmir 3210, Israel) and provided by drinking nipples. |
| | Details to be recorded into BSH-FRM-0004229. |
| Permissible and | No treatment other than the HN50 and F50 vaccination will be |
| non-permissible | administered during the study. Use of additives in food formula has |
| concomitant | no impact on the study (BSH-DOC-MTF-0003208). No antibiotic |
| veterinary care | treatment will be added during the period of the trial. |
| and therapy | |
| Environmental | Environmental control will be recorded daily in “daily check form” |
| control | BSH-FRM-0004229. The graph of the inactivated room temperature |
| | and pressure will be recorded and placed in the study report and will |
| | follow the procedures as described in AH-001. |
| Rearing of study | Study animals will be maintained at temperatures (using built-in |
| animals | heater with thermostat) within the guidelines for the age of bird (as |
| | detailed below), lightning according to BSH-SOP-0000221. |

Housing temperature according to poultry age:

| Temperature | Age in weeks | |
|---|---|---|
| 32 ± 3° C. | 1-4 | days |
| 31 ± 3° C. | 4-7 | days |
| 29 ± 3° C. | 2 | weeks |
| 27 ± 3° C. | 3 | weeks |
| 26 ± 3° C. | 4 | weeks |
| 25 ± 3° C. | 5-7 | weeks |

Animal Randomization

The day-old chicks intended for treatment groups from 1 to 12 were placed in a cardboard box and, from there, removed randomly and assigned to one of the test groups. Both male and female were selected for the trial. Each chick was identified by wing tagging on the vaccination day.

Blinding

The vaccinations, bleedings, and sera were blinded.

### Test Items

The raw materials were as follows.

NDV Genotype 7 Inactivated Vaccine (Batch A0022131005)

HN50 pure protein Phase 2 (batch MT555-6): Centrifuged once upon harvesting to remove mycelium, with PMSF added to the final concentration 1 mM; The supernatant was filtered through 0.45 μm filter before C-tag column purification. HN50 concentration of purification Batch no. 1 was 5.37 mg/ml.

HN50 ultra-filtrated material Phase 2 (batch MT569-6): Centrifuged once upon harvesting to remove mycelium, with PMSF added to the final 1 mM concentration. The supernatant was filtered through 0.45 μm filter before ultrafiltration on 10 kDa. The HN50 concentration was 0.65 mg/mL.

HN50 pure protein Phase 3 (batch MT641.6.1): Centrifuged once upon harvesting to remove mycelium, with PMSF added to the final 1 mM concentration; then the supernatant was filtered through 0.45 μm filter before C-tag column purification. HN50 concentration equal to 1.49 mg/mL.

F50 pure protein Phase 3 (batch MT642.6.1): Centrifuged once upon harvesting to remove mycelium, with PMSF added to the final 1 mM concentration; then the supernatant was filtered through 0.45 μm filter before C-tag column purification. HN50 concentration equal to 1.18 mg/mL.

Other emulsion raw materials were as follows: White mineral oil, for example, DRAKEOL; Sorbitol-S; Phosphate Buffered Saline (PBS); Sorbitol-T.

Vaccine Preparations

Methods to produce emulsions from raw materials are as follows.

M-1-3274: In a sterile hood, the aqueous phase was prepared by diluting 0.14 mL of HN50 into 4.26 mL PBS. 0.15 mL of Sorbital T was added, and the mixture incubated at 37° C. for 30 minutes with mixing/vortexing from time to time. 10 mL of mineral oil were mixed with 0.45 mL of Sorbital S. The emulsion will be homogenized in a Polytron at maximal speed for 1 minute.

M-1-3275: In a sterile hood, the aqueous phase was prepared by diluting 0.47 mL of HN50 into 3.93 mL PBS. 0.15 mL of Sorbital T was added, and the mixture incubated at 37° C. for 30 minutes with mixing/vortexing from time to time. 10 mL of mineral oil were mixed with 0.45 mL of Sorbital S. The emulsion was homogenized in a Polytron at maximal speed for 1 minute.

M-1-3276: In a sterile hood, the aqueous phase was prepared by diluting 0.93 mL of HN50 into 3.47 mL PBS. 0.15 mL of Sorbital T was added, and the mixture incubated at 37° C. for 30 minutes with mixing/vortexing from time to time. 10 mL of mineral oil were mixed with 0.45 mL of Sorbital S. The emulsion was homogenized in a Polytron at maximal speed for 1 minute.

M-1-3277: In a sterile hood, the aqueous phase was prepared by diluting 0.7 mL of HN50 into 2.2 mL PBS. 0.1 mL of Sorbital T was added, and the mixture incubated at 37° C. for 30 minutes with mixing/vortexing from time to time. 6.7 mL of mineral oil were mixed with 0.3 mL of Sorbital S. The emulsion was homogenized in a Polytron at maximal speed for 1 minute.

M-1-3278: In a sterile hood, the aqueous phase was prepared by diluting 2.5 mL of HN50 into 0.4 mL PBS. 0.1 mL of Sorbital T was added, and the mixture incubated at 37° C. for 30 minutes with mixing/vortexing from time to time. 6.7 mL of mineral oil were mixed with 0.3 mL of Sorbital S. The emulsion was homogenized in a Polytron at maximal speed for 1 minute.

M-1-3279: In a sterile hood, the aqueous phase was prepared by using 3.8 mL of HN50 into 2.2 mL PBS. 0.1 mL of Sorbital T was added, and the mixture was incubated at 37° C. for 30 minutes with mixing/vortexing from time to time. 6.7 mL of mineral oil were mixed with 0.3 mL of Sorbital S. The emulsion was homogenized in a Polytron at maximal speed for 1 minute.

M-1-3280: In a sterile hood, t the aqueous phase was prepared by diluting 0.5 mL of HN50 into 3.9 mL PBS. 0.15 mL of Sorbital T was added, and the mixture incubated at 37° C. for 30 minutes with mixing/vortexing from time to time. 10 mL of mineral oil were mixed with 0.45 mL of Sorbital S. The emulsion was homogenized in a Polytron at maximal speed for 1 minute.

M-1-3281: In a sterile hood, the aqueous phase was prepared by diluting 1.7 mL of HN50 into 2.7 mL PBS. 0.15 mL of Sorbital T was added, and the mixture incubated at 37° C. for 30 minutes with mixing/vortexing from time to time. 10 mL of mineral oil are mixed with 0.45 mL of Sorbital S. The emulsion was homogenized in a Polytron at maximal speed for 1 minute.

M-1-3282: In a sterile hood, the aqueous phase was prepared by diluting 3.4 mL of HN50 into 1 mL PBS. 0.15 mL of Sorbital T was added, and the mixture incubated at 37° C. for 30 minutes with mixing/vortexing from time to time. 10 mL of mineral oil were mixed with 0.45 mL of Sorbital S. The emulsion was homogenized in a Polytron at maximal speed for 1 minute.

M-1-3283: In a sterile hood, the aqueous phase was prepared by diluting 4.2 mL of F50 into 4.6 mL PBS. 0.3 mL of Sorbital T was added, and the mixture incubated at 37° C. for 30 minutes with mixing/vortexing from time to time. 20 mL of mineral oil wee mixed with 0.9 mL of Sorbital S. The emulsion was homogenized in a Polytron at maximal speed for 1 minute.

Vaccine formulations summaries are provided in Table 31.

TABLE 31
Vaccine Formulations Summary.

| | | DRAKEOL | Sorbital S | | | Sorbital T | Total |
|---|---|---|---|---|---|---|---|
| Group | Formula | (67%) | (3%) | *Antigen | PBS | (1%) | vol. |
| 2 | M-1-3274 | 10 mL | 0.45 mL | 0.14 mL | 4.26 mL | 0.15 mL | 15 mL |
| 3 | M-1-3275 | 10 mL | 0.45 mL | 0.47 mL | 3.93 mL | 0.15 mL | 15 mL |
| 4 | M-1-3276 | 10 mL | 0.45 mL | 0.93 mL | 3.47 mL | 0.15 mL | 15 mL |
| 5 | M-1-3277 | 6.7 mL | 0.3 mL | 0.7 mL | 2.2 mL | 0.1 mL | 10 mL |
| 6 | M-1-3278 | 6.7 mL | 0.3 mL | 2.5 mL | 0.4 mL | 0.1 mL | 10 mL |
| 7 | M-1-3279 | 6.7 mL | 0.3 mL | 3.8 mL | — | 0.1 mL | 10 mL |
| 8 | M-1-3280 | 10 mL | 0.45 mL | 0.5 mL | 3.9 mL | 0.15 mL | 15 mL |
| 9 | M-1-3281 | 10 mL | 0.45 mL | 1.7 mL | 2.7 mL | 0.15 mL | 15 mL |
| 10 | M-1-3282 | 10 mL | 0.45 mL | 3.4 mL | 1 mL | 0.15 mL | 15 mL |
| 11 | M-1-3283 | 20 mL | 0.9 mL | 4.2 mL | 4.6 mL | 0.30 mL | 30 mL |

*HN50 pure protein Phase2 = 5.37 mg/mL,
HN50 ultra-filtrated material = 0.65 mg/mL,
HN50 pure protein Phase 3 = 1.49 mg/mL,
F50 pure protein Phase 3 = 1.18 mg/mL.

Test Doses

At 21 days of age, all chicks from groups 1 to 11 were IM vaccinated into the pectoral muscle with dose volume of 0.3 mL.

### Methods

Test Methods—Immunogenicity Evaluation

a) Serology: at the vaccination day (antibody level at zero time), after 21 days from vaccination, blood samples were collected from each chicken, according to BSH-SOP-0000221. Further, the sera was separated according to BSH-QCM-0001842 and the antibody levels measured using HI test according to the BSH-FRM-0004280, in the laboratory with rNDV-LS-NDV-HN G7 and VH antigens.

For the F50 antigen (group 11), the immunogenicity evaluation was carried out by testing the sera on NDV-F ELISA kit (catalogue no. CK122-BioChek)

b) Challenge: This procedure was performed at Veterinary Services by IM injecting 10^{6.0}ELD₅₀/chicken of vvNDV gVII. After challenge, the birds were daily observed during at least 21 days in order to detect any morbidity and mortality by vvNDV genotype VII virus.

### Disease Management

Case was taken to avoid contamination between the experimental group and the negative control. When visiting the study animals, either to perform study procedures or animal husbandry, all study personnel attend to unvaccinated control group, then the vaccinated group. The following precautions were also taken:

Disposal of study animals: Disposal of dead birds during or at the end of the trial was conducted according to procedures outlined by BSH-SOP-0000231. Briefly, biological hazard waste including carcasses and excess materials was put into 2 bags and evacuated only to the biological hazard waste container bin in the yard. The carcasses that cannot be evacuated on the same day by the authorized company were kept in the dedicated freezer until the evacuation day.

Disposal of study products: Any excess test materials were put into 2 bags and evacuated only to biological hazard waste container.

Clothing: Gowning rules are according to BSH-SOP-0000232. According to this SOP in each entry to the inactivated vaccine department, each staff member removed personal clothing and put on overall and shoes belonging only to this department.

Post-mortem: In case of post-mortem, sampling occurred in the washing room, each staff member wore another overall and shoe cover according to BSH-SOP-0000232. Once the procedure was finished, the overall and shoe covers belonging to the washing room will be disposed as a biohazard according to BSH-SOP-0000231.

### Results

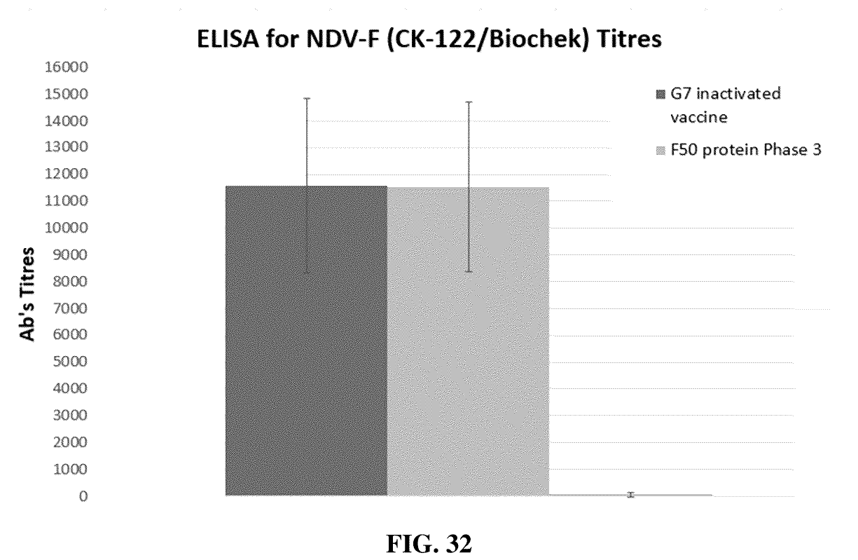

Expression results of the dose response of HN50 pure protein, 10 kDa ultrafiltered material after centrifugation and filtrate after 0.45 μM filtration; and the dose response of F50 pure protein from Phase 3 in prime and boost maximal doses are demonstrated in FIG. 32, which shows ELISA for NDV-F. Table 32 includes the raw ELISA NDV-F data.

TABLE 32
ELISA for NDV-F Raw Data

| | | | | | Log10 | | Titre | Titre |
|---|---|---|---|---|---|---|---|---|
| Group | Wing No. | Reading | Reading Av | S/P | Titre | Titre | Av | STDEV |
| NC | NC | 0.099 | | | | | | |
| NC | NC | 0.102 | 0.101 | | | | | |
| PC | PC | 0.326 | | | | | | |
| PC | PC | 0.317 | 0.322 | | | | | |
| G7 | 1031 | 1.312 | | 5.480 | 4.259 | 18145 | | |
| inactivated | | | | | | | | |
| vaccine | | | | | | | | |
| G7 | 1032 | 0.585 | | 2.190 | 3.860 | 7252 | | |
| inactivated | | | | | | | | |
| vaccine | | | | | | | | |
| G7 | 1033 | 0.892 | | 3.579 | 4.074 | 11852 | | |
| inactivated | | | | | | | | |
| vaccine | | | | | | | | |
| G7 | 1034 | 0.917 | | 3.692 | 4.087 | 12226 | | |
| inactivated | | | | | | | | |
| vaccine | | | | | | | | |
| G7 | 1035 | 0.816 | | 3.235 | 4.030 | 10713 | | |
| inactivated | | | | | | | | |
| vaccine | | | | | | | | |
| G7 | 1036 | 0.864 | | 3.452 | 4.058 | 11432 | | |
| inactivated | | | | | | | | |
| vaccine | | | | | | | | |
| G7 | 1037 | 0.628 | | 2.385 | 3.897 | 7896 | | |
| inactivated | | | | | | | | |
| vaccine | | | | | | | | |
| G7 | 1038 | 1.108 | | 4.557 | 4.179 | 15088 | | |
| inactivated | | | | | | | | |
| vaccine | | | | | | | | |
| G7 | 1039 | 0.61 | | 2.303 | 3.882 | 7627 | | |
| inactivated | | | | | | | | |
| vaccine | | | | | | | | |
| G7 | 1040 | 1.002 | | 4.077 | 4.130 | 13500 | | |
| inactivated | | | | | | | | |
| vaccine | | | | | | | | |
| G7 | 1041 | 0.735 | | 2.869 | 3.978 | 9499 | | |
| inactivated | | | | | | | | |
| vaccine | | | | | | | | |
| G7 | 1042 | 1.006 | 0.873 | 4.095 | 4.132 | 13560 | 11566 | 3255 |
| inactivated | | | | | | | | |
| vaccine | | | | | | | | |
| F protein | 1151 | 1.017 | | 4.145 | 4.138 | 13725 | | |
| (phase 3)- | | | | | | | | |
| 50 ug/dose | | | | | | | | |
| F protein | 1152 | 0.879 | | 3.520 | 4.067 | 11657 | | |
| (phase 3)- | | | | | | | | |
| 50 ug/dose | | | | | | | | |
| F protein | 1153 | 1.208 | | 5.009 | 4.220 | 16587 | | |
| (phase 3)- | | | | | | | | |
| 50 ug/dose | | | | | | | | |
| F protein | 1154 | 0.846 | | 3.371 | 4.048 | 11163 | | |
| (phase 3)- | | | | | | | | |
| 50 ug/dose | | | | | | | | |
| F protein | 1155 | 0.64 | | 2.439 | 3.907 | 8076 | | |
| (phase 3)- | | | | | | | | |
| 50 ug/dose | | | | | | | | |
| F protein | 1156 | 1.004 | | 4.086 | 4.131 | 13530 | | |
| (phase 3)- | | | | | | | | |
| 50 ug/dose | | | | | | | | |
| F protein | 1157 | 1.21 | | 5.018 | 4.221 | 16616 | | |
| (phase 3)- | | | | | | | | |
| 50 ug/dose | | | | | | | | |
| F protein | 1158 | 0.557 | | 2.063 | 3.835 | 6832 | | |
| (phase 3)- | | | | | | | | |
| 50 ug/dose | | | | | | | | |
| F protein | 1159 | 1.012 | | 4.122 | 4.135 | 13650 | | |
| (phase 3)- | | | | | | | | |
| 50 ug/dose | | | | | | | | |
| F protein | 1160 | 1.11 | | 4.566 | 4.179 | 15118 | | |
| (phase 3)- | | | | | | | | |
| 50 ug/dose | | | | | | | | |
| F protein | 1161 | 0.78 | | 3.072 | 4.007 | 10174 | | |
| (phase 3)- | | | | | | | | |
| 50 ug/dose | | | | | | | | |
| F protein | 1162 | 0.787 | | 3.104 | 4.012 | 10279 | | |
| (phase 3)- | | | | | | | | |
| 50 ug/dose | | | | | | | | |
| F protein | 1163 | 0.86 | | 3.434 | 4.056 | 11372 | | |
| (phase 3)- | | | | | | | | |
| 50 ug/dose | | | | | | | | |
| F protein | 1164 | 0.602 | | 2.267 | 3.875 | 7507 | | |
| (phase 3)- | | | | | | | | |
| 50 ug/dose | | | | | | | | |
| F protein | 1165 | 1.073 | | 4.398 | 4.163 | 14564 | | |
| (phase 3)- | | | | | | | | |
| 50 ug/dose | | | | | | | | |
| F protein | 1166 | 0.847 | | 3.376 | 4.048 | 11178 | | |
| (phase 3)- | | | | | | | | |
| 50 ug/dose | | | | | | | | |
| F protein | 1167 | 1.28 | | 5.335 | 4.247 | 17665 | | |
| (phase 3)- | | | | | | | | |
| 50 ug/dose | | | | | | | | |
| F protein | 1168 | 0.893 | | 3.584 | 4.074 | 11867 | | |
| (phase 3)- | | | | | | | | |
| 50 ug/dose | | | | | | | | |
| F protein | 1169 | 0.592 | | 2.222 | 3.867 | 7357 | | |
| (phase 3)- | | | | | | | | |
| 50 ug/dose | | | | | | | | |
| F protein | 1170 | 0.793 | | 3.131 | 4.016 | 10368 | | |
| (phase 3)- | | | | | | | | |
| 50 ug/dose | | | | | | | | |
| F protein | 1171 | 0.705 | | 2.733 | 3.957 | 9050 | | |
| (phase 3)- | | | | | | | | |
| 50 ug/dose | | | | | | | | |
| F protein | 1172 | 0.56 | | 2.077 | 3.837 | 6877 | | |
| (phase 3)- | | | | | | | | |
| 50 ug/dose | | | | | | | | |
| F protein | 1173 | 0.714 | | 2.774 | 3.963 | 9185 | | |
| (phase 3)- | | | | | | | | |
| 50 ug/dose | | | | | | | | |
| F protein | 1174 | 0.939 | 0.871 | 3.792 | 4.099 | 12556 | 11540 | 3173 |
| (phase 3)- | | | | | | | | |
| 50 ug/dose | | | | | | | | |
| Not | 1175 | 0.117 | | 0.072 | 2.380 | 240 | | |
| vaccinated | | | | | | | | |
| Not | 1176 | 0.102 | | 0.005 | 0.000 | 1 | | |
| vaccinated | | | | | | | | |
| Not | 1177 | 0.103 | | 0.009 | 1.477 | 30 | | |
| vaccinated | | | | | | | | |
| Not | 1178 | 0.116 | | 0.068 | 2.352 | 225 | | |
| vaccinated | | | | | | | | |
| Not | 1179 | 0.099 | | −0.009 | 0.000 | 1 | | |
| vaccinated | | | | | | | | |
| Not | 1180 | 0.096 | | −0.023 | 0.000 | 1 | | |
| vaccinated | | | | | | | | |
| Not | 1181 | 0.108 | | 0.032 | 2.021 | 105 | | |
| vaccinated | | | | | | | | |
| Not | 1182 | 0.098 | | −0.014 | 0.000 | 1 | | |
| vaccinated | | | | | | | | |
| Not | 1183 | 0.101 | | 0.000 | 0.000 | 1 | | |
| vaccinated | | | | | | | | |
| Not | 1184 | 0.114 | | 0.059 | 0.000 | 1 | | |
| vaccinated | | | | | | | | |
| Not | 1185 | 0.111 | | 0.045 | 0.000 | 1 | | |
| vaccinated | | | | | | | | |
| Not | 1186 | 0.095 | | −0.027 | 0.000 | 1 | | |
| vaccinated | | | | | | | | |
| Not | 1187 | 0.097 | | −0.018 | 0.000 | 1 | | |
| vaccinated | | | | | | | | |
| Not | 1188 | 0.102 | | 0.005 | 1.176 | 15 | | |
| vaccinated | | | | | | | | |
| Not | 1189 | 0.104 | | 0.014 | 1.653 | 45 | | |
| vaccinated | | | | | | | | |
| Not | 1190 | 0.11 | | 0.041 | 0.000 | 1 | | |
| vaccinated | | | | | | | | |
| Not | 1191 | 0.099 | | −0.009 | 0.000 | 1 | | |
| vaccinated | | | | | | | | |
| Not | 1192 | 0.111 | | 0.045 | 2.176 | 150 | | |
| vaccinated | | | | | | | | |
| Not | 1193 | 0.113 | | 0.054 | 0.000 | 1 | | |
| vaccinated | | | | | | | | |
| Not | 1194 | 0.098 | | −0.014 | 0.000 | 1 | | |
| vaccinated | | | | | | | | |
| Not | 1195 | 0.096 | | −0.023 | 0.000 | 1 | | |
| vaccinated | | | | | | | | |
| Not | 1196 | 0.096 | | −0.023 | 0.000 | 1 | | |
| vaccinated | | | | | | | | |
| Not | 1197 | 0.104 | | 0.014 | 0.000 | 1 | | |
| vaccinated | | | | | | | | |
| Not | 1198 | 0.099 | 0.104 | −0.009 | 0.000 | 1 | 34 | 71 |
| vaccinated | | | | | | | | |

### Example 6

### NDV PD50 and Dose Response for F50 Antigen Plus Stability Test for HN50 Batches in SPF Chickens

### Background

A previously discussed, Newcastle disease (ND) is a highly contagious viral disease that can affect all bird species. This enveloped virus (Avulavirus genus, Paramyoxoviridae family, Mononegavirales order, recently renamed as Avian Avulavirus or AAvV) has a marked genomic variability. Genotypes V, VI, VII and VIII (from class II) are predominant in different countries all over the world.

The clinical signs related to the infection are described as respiratory, gastrointestinal and neurologic signs which depend on the animal immune status and susceptibility and the strain involved. Depending on their pathogenicity, NDV viruses can be also classifies as: Velogenic Viscerotropic (ability to cause necro-haemorrhagic lesions in the intestine, cause up to 100% morbidity and mortality in naive chickens, vvNDV), Mesogenic (respiratory and neurological signs with moderate morbidity and mortality) and Lentogenic (minimal respiratory disease observed, especially when associated with secondary bacterial infection, used as live vaccines).

Inactivated ND vaccine (Nectiv Forte™) is used routinely to vaccinate chickens and turkeys. The vaccine provides slow response but longer duration of immunity. The vaccine formulation is water in oil emulsion and is based on mineral oil plus Montane/Montaneox as emulsifiers. To produce the NDV antigens for inactivated ND vaccines, the virus is propagated in embryonic egg. This vaccine is based on LaSota backbone expressing HN gene of NDV G7. The positive control in our trial will be NDV G7 vaccine.

C1 has been used to produce immunogenic recombinant antigens (HN or F proteins) in order to replace the ND vaccine production in embryonic eggs. This expression system provides high-yield, low cost, rapidly scalable, rVaccine-antigens and antibody gene expression platform in single use or stainless-steel bioreactors. It offers safe and effective antigens, generally recognized as safe (GRAS) certification from US FDA, and no negative clinical effects have been observed in animal studies. In previous example, the dose of 50 μg/0.3 mL F50 fused to T4 trimerization domain gave 100% protection against challenge with vvNDV (see Example 5). In addition, the vaccinated chickens gave equal antibody titres as the NDV G7 commercial vaccine.

### Objectives

The objectives of this study were to: (1) test the dose response and PD50 of F50 pure protein; (2) test the inhibitory effect of HN50 supernatant with F50 pure protein levels; and (3) test HN50 supernatant emulsion for stability.

### Study Design

Treatment Groups

195 SPF chicks were hatched in animal houses and separated into 17 groups. Groups 1-17 include approximately 12 chickens each. This includes sixteen vaccinated groups and one non-vaccinated control group. Groups 1 and 2 receive NDV G7 positive dose with its 1:50 dilution. Groups 3-7 and 9 receive PD50 trial for F50 pure protein (Phase 3). Groups 8-10 are for dose response trial for F50 pure protein (Phase 3). Groups 11-13 are for investigation of inhibitory effect of HN50 medium. Groups 14-16 are for stability test of stored vaccine batches. Finally, Group 17 is a non-vaccinated control group. Each group is allocated in the same rearing battery from day one. At 21 days of age, blood samples are taken randomly from the chicks before vaccination (time zero), the chickens are neck tagged from groups 1-17, and the birds in each vaccination group are vaccinated with one of the formulations by intramuscular (IM) injection with different volumes per chicken according to Table 33. Three weeks after vaccination at 42 days old, all the chicks in all groups are bled and the samples identified individually for further testing of the HI antibody levels against NDV. All chickens are sent for NDV challenge on day 43. The schedule of events is summarized in Table 34.

TABLE 33
Study Groups

| | | | Conc./ | | Chicken |
|---|---|---|---|---|---|
| Group | Description | Batch | dose | | per group |
| 1 | NDV G7 commercial batch | 22131006 | 0.3 | mL | 12 |
| 2 | NDV G7 commercial batch 1:50 | 22131006 | 6 | μL | 12 |

| | | | | |
|---|---|---|---|---|
| 3 | F50 pure protein (Phase3) 1:12.5 | M-1-3404 | 4 μg/24 μL | 12 |
| 4 | F50 pure protein (Phase3) 1:25 | M-1-3404 | 2 μg/12 μL | 12 |
| 5 | F50 pure protein (Phase3) 1:50 | M-1-3404 | l μg/6 μL | 12 |
| 6 | F50 pure protein (Phase3) 1:100 | M-1-3404 | 0.5 μg/3 μL | 12 |
| 7 | F50 pure protein (Phase3) 1:200 | M-1-3404 | 0.25 μg/1.5 μL | 12 |
| 8 | F50 pure protein (Phase3) | M-1-3402 | 15 μg/0.3 mL | 12 |
| 9 | F50 pure protein (Phase3) - Full dose | M-1-3404 | 50 μg/0.3 mL | 12 |
| 10 | F50 pure protein (Phase3) | M-1-3406 | 88 μg/0.3 mL | 12 |
| 11 | F50 pure protein (Phase3) in HN50 Sup | M-1-3407 | 15 μg/0.3 mL | 12 |
| 12 | F50 pure protein (Phase3) in HN50 Sup | M-1-3409 | 50 μg/0.3 mL | 12 |
| 13 | F50 pure protein (Phase3) in HN50 Sup | M-1-3411 | 88 μg/0.3 mL | 12 |
| 14 | HN50 Supernatant batch | M-1-3223 | 12.5 μg/0.3 mL | 12 |
| 15 | HN50 Supernatant batch | M-1-3227 | 100 μg/0.3 mL | 12 |
| 16 | HN50 Supernatant batch (M-569.6) | M-1-2921 | 15 μg/0.3 mL | 12 |
| 17 | Non-vaccinated | NA | NA | 12 |

Groups 1 and 2: NDV G7 positive dose with its 1:50 dilution.
Groups 3-7 and 9: PD50 trial for F50 pure protein (Phase 3).
Groups 8-10: Dose response trial for F50 pure protein (Phase 3).
Groups 11-13: Investigation for inhibitory effect of HN50 medium.
Groups 14-16: stability test for old batches.
Group 17: non-vaccinated control.

### Schedule of Events

TABLE 34
Trial Events

| Trial Day | Action |
|---|---|
| 1 | Hatching of SPF chicks. |
| 21 | Neck tag. |
| | Bleeding for serology time zero - 10 chickens randomly |
| | selected from the large group (10% sampling) |
| | Vaccination for groups 1-11. |
| 21-42 | Veterinary inspection for vaccination clinical signs |
| | evaluation. (According to BSH-FRM-0004233) |
| 42 | Bleeding for serology, separation by group. |
| 43 | Send the chickens for groups 1 to 17 to vvNDV challenge |
| | and end of the trial. |
| 44 | Challenge chickens from groups 1 to 17 to vvNDV challenge. |
| 44-58 | Monitor the chickens for NDV symptoms |

*Deviations of ±3 day were acceptable.

### Materials

Test System—Animals

The animals used in this study are day old Specified Pathogen Free (SPF) chicks, Gallus gallus spp., White Leghorn breed hatched from SPF eggs from an SPF flock. Both male and female chicks were used. In order to qualify for the study, the chicks have to originate from the same SPF flock and batch of eggs, be healthy and free of any signs of disease, and have a normal appearance and normal behavior.

All management and husbandry procedures were according to AH-001. Table shows details about the test system to be used in this study.

TABLE 35
Detailed Information of the Test System Performed

| | |
|---|---|
| Species: | Day old Specified Pathogen Free (SPF) chicks - Gallus gallus |
| | spp. White Leghorn breed will be hatched from SPF eggs from a SPF flock. |
| Supplier flock number | batch no.: S PAFAS, Flock: N112 Batch: 032932-2067 |
| Initial Age | One day old c hckens at day 0 (zero) |
| Serological Status | The CoA of the SPF embryonated eggs used, will be attached to the report. |
| Sex | Male or female |
| Origen | Embryonated SPF eggs will be sourced from a commercial |
| | supplier and incubated within the SPF hatchery at Phibro's Test |
| | Facility (according to BSH-INS-WI.-0005129) until enrolment |
| | onto the study at 18 days old embryo. Then, the well-developed |
| | embryos will be transferred to the Test Facility Test Site (with |
| | filled and signed BSH-FRM-0005102). Upon arrival to the Test |
| | Facility Test Site, the eggs will be placed into the hatchery |
| | according to BSH-SOP-0000231. |
| Hatching | According to BSH-SOP-0000221 and BSH-FRM-0005102. |
| | Hatching the embryonated SPF and transfer to the inactivated |
| | room. SPF chickens will be from the same SPF flock and batch of |
| | eggs. From the beginning through the duration of the study the |
| | birds will be subjected to daily inspection of the animal care and |
| | welfare office for the daily records (according to the BSH-FRM- |
| | 0004233). Only chickens showing normal behaviour (according |
| | to BSH-FRM-0004233) will be selected for the study |

Inclusion/Exclusion/Withdrawal Criteria

Acceptance of birds into the study was recorded into BSH-TMP-0004235. During the study, any sick or dead birds were examined by the responsible veterinarian surgeon and the cause recorded into BSH-FRM-0004234. In case of any morbidity/mortality unrelated to vaccination the birds were excluded from the analysis of results. Animals withdrawing were conducted according to BSH-SOP-0000231. The number of animals withdrawn prematurely from the studies and the reasons for such withdrawal were recorded and reported in the study report.

### Animal Management

The animal management and husbandry procedures are shown in Table 36.

TABLE 36
Animal Management

| | |
|---|---|
| Animal welfare | We declare and guarantee that all animal studies for rNDV-LS- |
| | NDV-HN G7 vaccine will be in line with the relevant legislation in |
| | EU (Directive 2010/63/EU) and Israel and in accordance with |
| | Israeli animal welfare legislation. |
| Housing | Study animals will be mixed and allocated in cages in the |
| | inactivated room, the whole period of study, in Phibro's Test |
| | Facility - test site at Har Tuv. |
| Food and water | Tap water and food will be available ad libitum. Food will be in |
| | the form of standard rations appropriate to the age of the birds: |
| | Standard chick diet (Tadmir 3210, Israel) and provided by drinking |
| | nipples. Details to be recorded into BSH-FRM-0004229. |
| Permissible and | No treatment other than the HN50 and F50 vaccination will be |
| non-permissible | administered during the study. Use of additives in food formula |
| concomitant | |
| veterinary care | has no impact on the study (BSH-DOC-MTF-0003208). No |
| and therapy | antibiotic treatment will be added during the period of the trial. |
| Environmental | Environmental control will be recorded daily in “daily check form” |
| control | BSH-FRM-0004229. The graph of the inactivated room |
| | temperature and pressure will be recorded and placed in the study |
| | report and will follow the procedures as described in BSH-SOP- |
| | 0000221. |
| Rearing of study | Study animals will be maintained at temperatures (using built-in |
| animals | heater with thermostat) within the guidelines for the age of bird (as |
| | detailed below), lightning according to BSH-SOP-0000221. |

Housing temperature according to poultry age:

| Temperature | Age in weeks | |
|---|---|---|
| 32 ± 3° C. | 1-4 | days |
| 31 ± 3° C. | 4-7 | days |
| 29 ± 3° C. | 2 | weeks |
| 27 ± 3° C. | 3 | weeks |
| 26 ± 3° C. | 4 | weeks |
| 25 ± 3° C. | 5-7 | weeks |

### Animal Randomization

The day-old chicks intended for treatment groups from 1 to 17 will be placed in a cardboard box and, from there, they will be removed randomly and assigned to one of the groups. Both male and female will be selected for the trial. Each chick will be identified by wing tagging at the vaccination day.

Blinding

The vaccinations, bleedings, and sera was blinded.

### Test Items

The raw materials were as follows.

NDV Genotype 7 Inactivated Vaccine (Batch 22131006)

F50 pure protein Phase 3 (batch MT642.6.1): centrifuged once upon harvesting to remove mycelium, with PMSF added to the final concentration 1 mM. Then the supernatant was filtered through 0.45 μm filter before C-tag column purification. F50 concentration equal to 1.18 mg/ml.

HN50 supernatant (Batch MT443.6): samples were passed through 0.45 filter to remove cell debris. Total protein 20.7 mg/ml; HN50 concentration−1.05 mg/Ml.

HN50 supernatant (Batch M-1-3223): HN50 C1 fermentation supernatant (batch MT569-6) was designed to have 12.5 μg/dose from Phase 2 material for SP-791.

HN50 supernatant (Batch M-1-3227): HN50 C1 fermentation supernatant (batch MT569-6) was designed to have 95 μg/dose from Phase 2 material for SP-791.

HN50 supernatant (Batch M-1-2921): HN50 C1 fermentation supernatant (batch MT569-6) was designed to have 15 μg/dose from Phase 2 material for SP-779.

Other emulsion raw materials included: white mineral oil, for example, DRAKEOL; Sorbitol-S; Phosphate-buffered Saline (PBS); and Sorbitol-T.

Vaccine Preparations

Vaccines were prepared using the following procedures. Table 37 provides a vaccination formulations summary.

M-1-3402: In a sterile hood, the aqueous phase was prepared by diluting 0.21 mL of F50 into 1.24 mL PBS. 0.05 mL of Sorbital T was added, and the mixture incubated at 37° C. for 30 minutes with mixing/vortexing from time to time. 3.35 mL of mineral oil were mixed with 0.15 mL of Sorbital S. The emulsion was homogenized in a Polytron at maximal speed for 1 minutes.

M-1-3404: In a sterile hood, the aqueous phase was prepared by diluting 1.4 mL of F50 into 1.6 mL PBS. 0.1 mL of Sorbital T was added, and the mixture incubated at 37° C. for 30 minutes with mixing/vortexing from time to time. 6.7 mL of mineral oil were mixed with 0.3 mL of Sorbital S. The emulsion was homogenized in a Polytron at maximal speed for 1 minute.

M-1-3406: In a sterile hood, the aqueous phase was prepared by diluting 1.2 mL of F50 into 0.3 mL PBS. 0.05 mL of Sorbital T was added, and the mixture incubated at 37° C. for 30 minutes with mixing/vortexing from time to time. 3.35 mL of mineral oil were mixed with 0.15 mL of Sorbital S. The emulsion was homogenized in a Polytron at maximal speed for 1 minute.

M-1-3407: In a sterile hood, the aqueous phase was prepared by diluting 0.21 mL of F50 into 1.24 mL HN50 SUP (MT-443.6). 0.05 mL of Sorbital T was added, and the mixture incubated at 37° C. for 30 minutes with mixing/vortexing from time to time. 3.35 mL of mineral oil were mixed with 0.15 mL of Sorbital S. The emulsion was homogenized in a Polytron at maximal speed for 1 minute.

M-1-3409: In a sterile hood, the aqueous phase was prepared by diluting 0.7 mL of F50 into 0.8 mL HN50 SUP (MT-443.6). 0.05 mL of Sorbitol T was added, and the mixture incubated at 37° C. for 30 minutes with mixing/vortexing from time to time. 3.35 mL of mineral oil were mixed with 0.15 mL of Sorbitol S. The emulsion was homogenized in a Polytron at maximal speed for 1 minute.

M-1-3411: In a sterile hood, the aqueous phase was prepared by diluting 1.2 mL of F50 into 0.3 mL HN50 SUP (MT-443.6). 0.05 mL of Sorbitol T was added, and the mixture incubated at 37° C. for 30 minutes with mixing/vortexing from time to time. 3.35 mL of mineral oil were mixed with 0.15 mL of Sorbitol S. The emulsion was homogenized in a Polytron at maximal speed for 1 minute.

TABLE 37
Vaccine Formulations Summary

| | Dose | | | Sorbitol- | | | | |
|---|---|---|---|---|---|---|---|---|
| | (μg/0.3 | | DRAKEOL | S | | PBS/ | Sorbitol- | Total |
| Group | mL) | Formula | (67%) | (3%) | *Antigen | SUP | T (1%) | vol. |
| 8 | 15 | M-1- | 3.35 mL | 0.15 mL | 0.21 mL | 1.24 | 0.05 mL | 5 mL |
| | | 3402 | | | | mL | | |
| 9 | 50 | M-1- | 6.7 mL | 0.3 mL | 1.4 mL | 1.6 | 0.1 mL | 10 mL |
| | | 3404 | | | | mL | | |
| 10 | 88 | M-1- | 3.35 mL | 0.15 mL | 1.2 mL | 0.3 | 0.05 mL | 5 mL |
| | | 3406 | | | | mL | | |
| 11 | 15 | M-1- | 3.35 mL | 0.15 mL | 0.21 mL | 1.24 | 0.05 mL | 5 mL |
| | | 3407 | | | | mL | | |
| 12 | 50 | M-1- | 3.35 mL | 0.15 mL | 0.7 mL | 0.8 | 0.05 mL | 5 mL |
| | | 3409 | | | | mL | | |
| 13 | 88 | M-1- | 3.35 mL | 0.15 mL | 1.2 mL | 0.3 | 0.05 mL | 5 mL |
| | | 3411 | | | | mL | | |

*F50 pure protein Phase 3 = 1.18 mg/mL.

Test Doses

At 21 days of age, all chicks from groups 1 to 16 were IM vaccinated into the pectoral muscle with dose volumes as shown in Table 33.

### Methods

Test Method: Immunogenicity Evaluation

a) Serology: at the vaccination day (antibody level at zero time), after 21 days from vaccination, the blood samples were collected from each chicken in all groups, according to BSH-SOP-0000221.

Further, the sera were separated according to BSH-QCM-0001842 and the antibody levels measured for groups 1, 2, 14-17 using HI test according to the BSH-FRM-0004280, in the laboratory with rNDV-LS-NDV-HN G7 antigen.

For the F50 antigen in groups 1-13 and 17), the immunogenicity evaluation was carried out by testing the sera on NDV-F ELISA kit (catalogue no. CK122-BioChek)

b) Challenge: This procedure was performed at Veterinary Services by IM injecting 10^{6.0}ELD50/chicken of vvNDV gVII. After challenge, the birds were observed daily during at least 14 days in order to detect any morbidity and mortality by vvNDV genotype VII virus.

### Statistical Analysis

The PD50 was determined by the dose of a vaccine that may be expected to protect 50% of the chickens against the challenge dose of NDV. The PD50 also reflects the correlation between the antigen quantity, serological response, and protection from challenge.

By using the European Pharmacopoeia standards 10.2, monograph 0870, paragraph 2-3-2 as a guide regarding vaccine (Inactivated) Immunogenicity for Newcastle disease vaccine, the vaccine should comply with the PD50 test (50% protective dose) and should not be less than 50 percent of the recommended dose, while the lower confidence limit is 35 PD50 per dose. This means that the NDV vaccine must contain a minimum of 50 PD50 in one recommended dose, and the calculation is performed by using the Reed and Muench method as presented below:

$$Proportionate distance ( P D ) = \frac{\% of survivors next above 50 \% - 50 \%}{\begin{matrix}\% of survivors next above 50 \% - \\ \% of survivors next lower 50 \%\end{matrix}}$$

The log of the 50% endpoint=dilution above 50%−(PD*log dilution factor) PD50=10−x, where x is the log of the 50% endpoint. The statistical difference between each group based on the PD50 results will be determined by using the Two Way Anova model measurements design with JPM 12.0.1 software.

Assessments and Evaluable Parameter

All birds from negative control (group 17) should die within 6 days of challenge. The vaccines comply with the test if the smallest dose stated in a dose of 0.3 mL corresponds to not less than 50 PD50 and the lower confidence limit is not less than 35 PD50 per dose. If the lower confidence limit is less than 35 PD50 per dose, the test was repeated. The vaccine must contain not less than 50 PD50 in the repeat test.

### Disease Management

Care was taken to avoid contamination between the experimental group and the negative control according to BSH-SOP-0000231. When visiting the study animals, either to perform study procedures or animal husbandry, all study personnel always attended the unvaccinated control group, and then the vaccinated group.

Disposal of study products: Any excess test materials were put into 2 bags and evacuated only to biological hazard waste container.

Clothing: Gowning rules are under BSH-SOP-0000232. According to this SOP in each entry to the inactivated vaccine department, each staff member removed personal clothing and put on overall and shoes belonging only to this department.

Postmortem: In case of post-mortem, sampling occurred in the washing room, and each staff member wears another overall and shoe cover according to BSH-SOP-0000232. Once the procedure was finished, the overall and shoe covers belonging to the washing room will be disposed as a biohazard according to BSH-SOP-0000231.

### Results

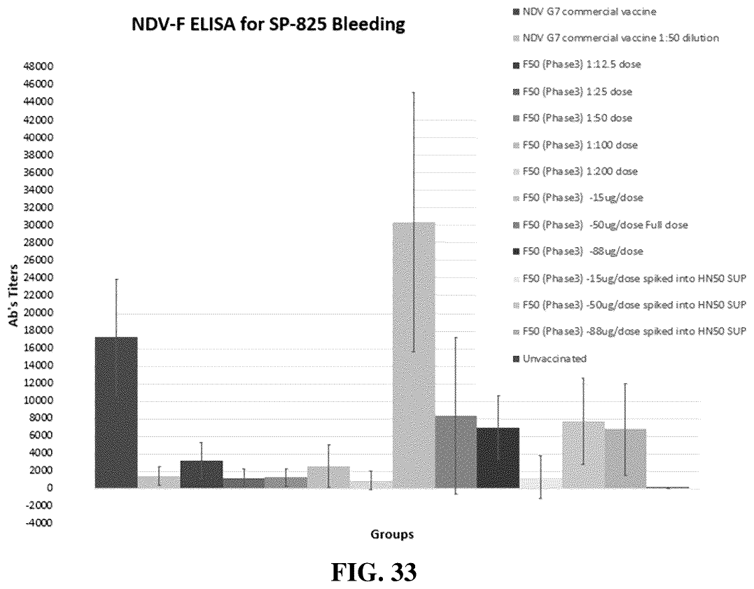

Dose response for F50 antigens and the stability test results are shown in FIG. 33 and Table 38 includes the raw data of NDV-F ELIA.

TABLE 38
Raw Data

| | | | | | Log10 | | Titre | Titre |
|---|---|---|---|---|---|---|---|---|
| Group | Wing no. | Reading | Reading Av | S/P | Titre | Titre | Av | STDEV |
| NC | NC | 0.127 | | | | | | |
| NC | NC | 0.123 | 0.125 | | | | | |
| PC | PC | 0.29 | | | | | | |
| PC | PC | 0.289 | 0.290 | | | | | |
| NDV G7 | 4201 | 1.001 | | 5.309 | 4.245 | 17580 | | |
| commercial | | | | | | | | |
| vaccine | | | | | | | | |
| NDV G7 | 4202 | 0.988 | | 5.230 | 4.239 | 17319 | | |
| commercial | | | | | | | | |
| vaccine | | | | | | | | |
| NDV G7 | 4203 | 1.436 | | 7.945 | 4.420 | 26310 | | |
| commercial | | | | | | | | |
| vaccine | | | | | | | | |
| NDV G7 | 4204 | 1.388 | | 7.655 | 4.404 | 25347 | | |
| commercial | | | | | | | | |
| vaccine | | | | | | | | |
| NDV G7 | 4205 | 0.827 | | 4.255 | 4.149 | 14088 | | |
| commercial | | | | | | | | |
| vaccine | | | | | | | | |
| NDV G7 | 4206 | 1.416 | | 7.824 | 4.413 | 25909 | | |
| commercial | | | | | | | | |
| vaccine | | | | | | | | |
| NDV G7 | 4207 | 0.822 | | 4.224 | 4.146 | 13988 | | |
| commercial | | | | | | | | |
| vaccine | | | | | | | | |
| NDV G7 | 4208 | 0.48 | | 2.152 | 3.853 | 7124 | | |
| commercial | | | | | | | | |
| vaccine | | | | | | | | |
| NDV G7 | 4209 | 1.227 | | 6.679 | 4.345 | 22116 | | |
| commercial | | | | | | | | |
| vaccine | | | | | | | | |
| NDV G7 | 4210 | 0.833 | | 4.291 | 4.153 | 14209 | | |
| commercial | | | | | | | | |
| vaccine | | | | | | | | |
| NDV G7 | 4211 | 0.484 | | 2.176 | 3.858 | 7205 | | |
| commercial | | | | | | | | |
| vaccine | | | | | | | | |
| NDV G7 | 4212 | 0.898 | 0.983 | 4.685 | 4.191 | 15513 | 17226 | 6625 |
| commercial | | | | | | | | |
| vaccine | | | | | | | | |
| NDV G7 | 4213 | 0.278 | | 0.927 | 3.487 | 3070 | | |
| commercial | | | | | | | | |
| vaccine | | | | | | | | |
| 1:50 | | | | | | | | |
| dilution | | | | | | | | |
| NDV G7 | 4214 | 0.128 | | 0.018 | 1.780 | 60 | | |
| commercial | | | | | | | | |
| vaccine | | | | | | | | |
| 1:50 | | | | | | | | |
| dilution | | | | | | | | |
| NDV G7 | 4215 | 0.151 | | 0.158 | 2.717 | 522 | | |
| commercial | | | | | | | | |
| vaccine | | | | | | | | |
| 1:50 | | | | | | | | |
| dilution | | | | | | | | |
| NDV G7 | 4216 | 0.14 | | 0.091 | 2.479 | 301 | | |
| commercial | | | | | | | | |
| vaccine | | | | | | | | |
| 1:50 | | | | | | | | |
| dilution | | | | | | | | |
| NDV G7 | 4217 | 0.15 | | 0.152 | 2.700 | 502 | | |
| commercial | | | | | | | | |
| vaccine | | | | | | | | |
| 1:50 | | | | | | | | |
| dilution | | | | | | | | |
| NDV G7 | 4218 | 0.259 | | 0.812 | 3.430 | 2689 | | |
| commercial | | | | | | | | |
| vaccine | | | | | | | | |
| 1:50 | | | | | | | | |
| dilution | | | | | | | | |
| NDV G7 | 4219 | 0.173 | | 0.291 | 2.984 | 963 | | |
| commercial | | | | | | | | |
| vaccine | | | | | | | | |
| 1:50 | | | | | | | | |
| dilution | | | | | | | | |
| NDV G7 | 4220 | 0.208 | | 0.503 | 3.222 | 1666 | | |
| commercial | | | | | | | | |
| vaccine | | | | | | | | |
| 1:50 | | | | | | | | |
| dilution | | | | | | | | |
| NDV G7 | 4221 | 0.247 | | 0.739 | 3.389 | 2448 | | |
| commercial | | | | | | | | |
| vaccine | | | | | | | | |
| 1:50 | | | | | | | | |
| dilution | | | | | | | | |
| NDV G7 | 4222 | 0.233 | | 0.655 | 3.336 | 2167 | | |
| commercial | | | | | | | | |
| vaccine | | | | | | | | |
| 1:50 | | | | | | | | |
| dilution | | | | | | | | |
| NDV G7 | 4223 | 0.236 | | 0.673 | 3.348 | 2228 | | |
| commercial | | | | | | | | |
| vaccine | | | | | | | | |
| 1:50 | | | | | | | | |
| dilution | | | | | | | | |
| NDV G7 | 4224 | 0.139 | 0.195 | 0.085 | 2.449 | 281 | 1408 | 1084 |
| commercial | | | | | | | | |
| vaccine | | | | | | | | |
| 1:50 | | | | | | | | |
| dilution | | | | | | | | |
| F50 | 4225 | 0.153 | | 0.170 | 2.750 | 562 | | |
| (Phase3) | | | | | | | | |
| 1:12.5 dose | | | | | | | | |
| F50 | 4226 | 0.303 | | 1.079 | 3.553 | 3572 | | |
| (Phase3) | | | | | | | | |
| 1:12.5 dose | | | | | | | | |
| F50 | 4227 | 0.178 | | 0.321 | 3.027 | 1064 | | |
| (Phase3) | | | | | | | | |
| 1:12.5 dose | | | | | | | | |
| F50 | 4228 | 0.235 | | 0.667 | 3.344 | 2208 | | |
| (Phase3) | | | | | | | | |
| 1:12.5 dose | | | | | | | | |
| F50 | 4229 | 0.226 | | 0.612 | 3.307 | 2027 | | |
| (Phase3) | | | | | | | | |
| 1:12.5 dose | | | | | | | | |
| F50 | 4230 | 0.202 | | 0.467 | 3.189 | 1545 | | |
| (Phase3) | | | | | | | | |
| 1:12.5 dose | | | | | | | | |
| F50 | 4231 | 0.25 | | 0.758 | 3.399 | 2509 | | |
| (Phase3) | | | | | | | | |
| 1:12.5 dose | | | | | | | | |
| F50 | 4232 | 0.287 | | 0.982 | 3.512 | 3251 | | |
| (Phase3) | | | | | | | | |
| 1:12.5 dose | | | | | | | | |
| F50 | 4233 | 0.437 | | 1.891 | 3.797 | 6261 | | |
| (Phase3) | | | | | | | | |
| 1:12.5 dose | | | | | | | | |
| F50 | 4234 | 0.374 | | 1.509 | 3.699 | 4997 | | |
| (Phase3) | | | | | | | | |
| 1:12.5 dose | | | | | | | | |
| F50 | 4235 | 0.266 | | 0.855 | 3.452 | 2830 | | |
| (Phase3) | | | | | | | | |
| 1:12.5 dose | | | | | | | | |
| F50 | 4236 | 0.476 | 0.282 | 2.127 | 3.848 | 7044 | 3156 | 2017 |
| (Phase3) | | | | | | | | |
| 1:12.5 dose | | | | | | | | |
| F50 | 4237 | 0.228 | | 0.624 | 3.315 | 2067 | | |
| (Phase3) | | | | | | | | |
| 1:25 dose | | | | | | | | |
| F50 | 4238 | 0.152 | | 0.164 | 2.734 | 542 | | |
| (Phase3) | | | | | | | | |
| 1:25 dose | | | | | | | | |
| F50 | 4239 | 0.172 | | 0.285 | 2.975 | 943 | | |
| (Phase3) | | | | | | | | |
| 1:25 dose | | | | | | | | |
| F50 | 4240 | 0.286 | | 0.976 | 3.509 | 3231 | | |
| (Phase3) | | | | | | | | |
| 1:25 dose | | | | | | | | |
| F50 | 4241 | 0.137 | | 0.073 | 2.382 | 241 | | |
| (Phase3) | | | | | | | | |
| 1:25 dose | | | | | | | | |
| F50 | 4242 | 0.225 | | 0.606 | 3.303 | 2007 | | |
| (Phase3) | | | | | | | | |
| 1:25 dose | | | | | | | | |
| F50 | 4243 | 0.149 | | 0.145 | 2.683 | 482 | | |
| (Phase3) | | | | | | | | |
| 1:25 dose | | | | | | | | |
| F50 | 4244 | 0.153 | | 0.170 | 2.750 | 562 | | |
| (Phase3) | | | | | | | | |
| 1:25 dose | | | | | | | | |
| F50 | 4245 | 0.179 | | 0.327 | 3.035 | 1084 | | |
| (Phase3) | | | | | | | | |
| 1:25 dose | | | | | | | | |
| F50 | 4246 | 0.241 | | 0.703 | 3.367 | 2328 | | |
| (Phase3) | | | | | | | | |
| 1:25 dose | | | | | | | | |
| F50 | 4247 | 0.143 | | 0.109 | 2.558 | 361 | | |
| (Phase3) | | | | | | | | |
| 1:25 dose | | | | | | | | |
| F50 | 4248 | 0.165 | 0.186 | 0.242 | 2.905 | 803 | 1221 | 954 |
| (Phase3) | | | | | | | | |
| 1:25 dose | | | | | | | | |
| F50 | 4249 | 0.257 | | 0.800 | 3.423 | 2649 | | |
| (Phase3) | | | | | | | | |
| 1:50 dose | | | | | | | | |
| F50 | 4250 | 0.22 | | 0.576 | 3.280 | 1907 | | |
| (Phase3) | | | | | | | | |
| 1:50 dose | | | | | | | | |
| F50 | 4251 | 0.232 | | 0.648 | 3.332 | 2147 | | |
| (Phase3) | | | | | | | | |
| 1:50 dose | | | | | | | | |
| F50 | 4252 | 0.129 | | 0.024 | 1.905 | 80 | | |
| (Phase3) | | | | | | | | |
| 1:50 dose | | | | | | | | |
| F50 | 4253 | 0.179 | | 0.327 | 3.035 | 1084 | | |
| (Phase3) | | | | | | | | |
| 1:50 dose | | | | | | | | |
| F50 | 4254 | 0.148 | | 0.139 | 2.664 | 462 | | |
| (Phase3) | | | | | | | | |
| 1:50 dose | | | | | | | | |
| F50 | 4255 | 0.211 | | 0.521 | 3.237 | 1726 | | |
| (Phase3) | | | | | | | | |
| 1:50 dose | | | | | | | | |
| F50 | 4256 | 0.133 | | 0.048 | 2.206 | 161 | | |
| (Phase3) | | | | | | | | |
| 1:50 dose | | | | | | | | |
| F50 | 4257 | 0.124 | | 0.000 | 0.000 | 1 | | |
| (Phase3) | | | | | | | | |
| 1:50 dose | | | | | | | | |
| F50 | 4258 | 0.18 | | 0.333 | 3.043 | 1104 | | |
| (Phase3) | | | | | | | | |
| 1:50 dose | | | | | | | | |
| F50 | 4259 | 0.184 | | 0.358 | 3.073 | 1184 | | |
| (Phase3) | | | | | | | | |
| 1:50 dose | | | | | | | | |
| F50 | 4260 | 0.261 | 0.188 | 0.824 | 3.436 | 2729 | 1269 | 972 |
| (Phase3) | | | | | | | | |
| 1:50 dose | | | | | | | | |
| F50 | 4261 | 0.328 | | 1.230 | 3.610 | 4074 | | |
| (Phase3) | | | | | | | | |
| 1:100 dose | | | | | | | | |
| F50 | 4262 | 0.518 | | 2.382 | 3.897 | 7887 | | |
| (Phase3) | | | | | | | | |
| 1:100 dose | | | | | | | | |
| F50 | 4263 | 0.445 | | 1.939 | 3.808 | 6422 | | |
| (Phase3) | | | | | | | | |
| 1:100 dose | | | | | | | | |
| F50 | 4264 | 0.143 | | 0.109 | 2.558 | 361 | | |
| (Phase3) | | | | | | | | |
| 1:100 dose | | | | | | | | |
| F50 | 4265 | 0.2 | | 0.455 | 3.178 | 1505 | | |
| (Phase3) | | | | | | | | |
| 1:100 dose | | | | | | | | |
| F50 | 4266 | 0.165 | | 0.242 | 2.905 | 803 | | |
| (Phase3) | | | | | | | | |
| 1:100 dose | | | | | | | | |
| F50 | 4267 | 0.258 | | 0.806 | 3.426 | 2669 | | |
| (Phase3) | | | | | | | | |
| 1:100 dose | | | | | | | | |
| F50 | 4268 | 0.124 | | 0.000 | 0.000 | 1 | | |
| (Phase3) | | | | | | | | |
| 1:100 dose | | | | | | | | |
| F50 | 4269 | 0.264 | | 0.842 | 3.446 | 2790 | | |
| (Phase3) | | | | | | | | |
| 1:100 dose | | | | | | | | |
| F50 | 4270 | 0.212 | | 0.527 | 3.242 | 1746 | | |
| (Phase3) | | | | | | | | |
| 1:100 dose | | | | | | | | |
| F50 | 4271 | 0.142 | | 0.103 | 2.533 | 341 | | |
| (Phase3) | | | | | | | | |
| 1:100 dose | | | | | | | | |
| F50 | 4272 | 0.217 | 0.251 | 0.558 | 3.266 | 1846 | 2537 | 2474 |
| (Phase3) | | | | | | | | |
| 1:100 dose | | | | | | | | |
| F50 | 4273 | 0.173 | | 0.291 | 2.984 | 963 | | |
| (Phase3) | | | | | | | | |
| 1:200 dose | | | | | | | | |
| F50 | 4274 | 0.139 | | 0.085 | 2.449 | 281 | | |
| (Phase3) | | | | | | | | |
| 1:200 dose | | | | | | | | |
| F50 | 4275 | 0.139 | | 0.085 | 2.449 | 281 | | |
| (Phase3) | | | | | | | | |
| 1:200 dose | | | | | | | | |
| F50 | 4276 | 0.148 | | 0.139 | 2.664 | 462 | | |
| (Phase3) | | | | | | | | |
| 1:200 dose | | | | | | | | |
| F50 | 4277 | 0.129 | | 0.024 | 1.905 | 80 | | |
| (Phase3) | | | | | | | | |
| 1:200 dose | | | | | | | | |
| F50 | 4278 | 0.145 | | 0.121 | 2.604 | 401 | | |
| (Phase3) | | | | | | | | |
| 1:200 dose | | | | | | | | |
| F50 | 4279 | 0.174 | | 0.297 | 2.993 | 983 | | |
| (Phase3) | | | | | | | | |
| 1:200 dose | | | | | | | | |
| F50 | 4280 | 0.124 | | 0.000 | 0.000 | 1 | | |
| (Phase3) | | | | | | | | |
| 1:200 dose | | | | | | | | |
| F50 | 4281 | 0.162 | | 0.224 | 2.871 | 743 | | |
| (Phase3) | | | | | | | | |
| 1:200 dose | | | | | | | | |
| F50 | 4282 | 0.206 | | 0.491 | 3.211 | 1626 | | |
| (Phase3) | | | | | | | | |
| 1:200 dose | | | | | | | | |
| F50 | 4283 | 0.238 | | 0.685 | 3.356 | 2268 | | |
| (Phase3) | | | | | | | | |
| 1:200 dose | | | | | | | | |
| F50 | 4284 | 0.305 | 0.174 | 1.091 | 3.558 | 3612 | 975 | 1062 |
| (Phase3) | | | | | | | | |
| 1:200 dose | | | | | | | | |
| F50 | 4285 | 2.471 | | 14.218 | 4.673 | 47081 | | |
| (Phase3)- | | | | | | | | |
| 15 ug/dose | | | | | | | | |
| F50 | 4286 | 2.68 | | 15.485 | 4.710 | 51275 | | |
| (Phase3)- | | | | | | | | |
| 15 ug/dose | | | | | | | | |
| F50 | 4287 | 2.453 | | 14.109 | 4.669 | 46720 | | |
| (Phase3)- | | | | | | | | |
| 15 ug/dose | | | | | | | | |
| F50 | 4288 | 1.586 | | 8.855 | 4.467 | 29320 | | |
| (Phase3)- | | | | | | | | |
| 15 ug/dose | | | | | | | | |
| F50 | 4289 | 0.758 | | 3.836 | 4.104 | 12703 | | |
| (Phase3)- | | | | | | | | |
| 15 ug/dose | | | | | | | | |
| F50 | 4290 | 1.413 | | 7.806 | 4.412 | 25848 | | |
| (Phase3)- | | | | | | | | |
| 15 ug/dose | | | | | | | | |
| F50 | 4291 | 0.53 | | 2.455 | 3.910 | 8128 | | |
| (Phase3)- | | | | | | | | |
| 15 ug/dose | | | | | | | | |
| F50 | 4292 | 1.256 | | 6.855 | 4.356 | 22698 | | |
| (Phase3)- | | | | | | | | |
| 15 ug/dose | | | | | | | | |
| F50 | 4293 | 1.318 | | 7.230 | 4.379 | 23942 | | |
| (Phase3)- | | | | | | | | |
| 15 ug/dose | | | | | | | | |
| F50 | 4294 | 0.917 | | 4.800 | 4.201 | 15894 | | |
| (Phase3)- | | | | | | | | |
| 15 ug/dose | | | | | | | | |
| F50 | 4295 | 1.851 | | 10.461 | 4.540 | 34638 | | |
| (Phase3)- | | | | | | | | |
| 15 ug/dose | | | | | | | | |
| F50 | 4296 | 2.443 | 1.640 | 14.048 | 4.668 | 46519 | 30397 | 14773 |
| (Phase3)- | | | | | | | | |
| 15 ug/dose | | | | | | | | |

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A transgenic fungal cell, comprising a recombinant gene comprising a fungal promoter operably linked to a nucleic acid sequence encoding an antigenic peptide derived from Newcastle Disease virus.

2. The transgenic fungal cell of claim 1, wherein the fungal cell is Thermothelomyces heterothallica.

3. The transgenic fungal cell of claim 2, wherein the Thermothelomyces heterothallica is a strain selected from DNL155, DNL157, DNL159, M5355, or M5739.

4. The transgenic fungal cell of claim 2, wherein the Thermothelomyces heterothallica is a glycoengineered strain producing Man3, G0, or G2 glycans.

5. The transgenic fungal cell of claim 1, wherein the antigenic peptide is a Newcastle Disease virus F protein, HN protein, or any combination thereof.

6. The transgenic fungal cell of claim 1, wherein the recombinant gene has a nucleic acid sequence encoding an antigenic peptide comprising a sequence of at least 90% similarity to SEQ ID NOs: 5, 8, 11, 14, 17, 20, 23, 26, 44, 45, or combinations thereof.

7. The transgenic fungal cell of claim 1, wherein the recombinant gene has a nucleic acid sequence corresponding to an amino acid sequence of at least 90% similarity to SEQ ID NOs: 3, 4, 6, 7, 9, 10, 12, 13, 15, 16, 18, 19, 21, 22, 24, 25, 27, 28, 30, 31, 32, or combinations thereof.

8. The transgenic fungal cell of claim 1, wherein the recombinant gene is transformed into the fungal cell via an episomal vector corresponding to the nucleic acid sequence of SEQ ID NOs: 29, 34, 36, or 37.

9. The transgenic fungal cell of any of claim 1, further comprising a signal sequence corresponding to an amino acid sequence of at least 90% similarity to SEQ ID NO: 1.

10. The transgenic fungal cell of claim 1, wherein the fungal promoter is bgl8.

11. A method for producing a recombinant antigenic peptide, comprising:
fermenting a transgenic fungal cell comprising a recombinant gene comprising a fungal promoter operably linked to a nucleic acid sequence encoding an antigenic peptide derived from Newcastle Disease virus in a bioreactor with a medium, and wherein the transgenic fungal cell is Thermothelomyces heterothallica;
collecting the recombinant antigenic protein produced during the fermentation; and
purifying the recombinant antigenic protein.

12. The method of claim 11, wherein the bioreactor has a temperature in the range of 20° C. to 40° C.

13. The method of claim 11, wherein the Thermothelomyces heterothallica is a strain selected from DNL155, DNL157, DNL159, M5355, or M5739.

14. The method of claim 11, wherein the Thermothelomyces heterothallica is a glycoengineered strain producing Man₃, G0, or G2 glycans.

15. The method of claim 11, wherein the recombinant gene is transformed into the fungal cell via an episomal vector corresponding to the nucleic acid sequence of SEQ ID NOs: 29, 34, 36, or 37.

16. The method of claim 11, wherein the recombinant gene has a nucleic acid sequence encoding an antigenic peptide comprising a sequence of at least 90% similarity to SEQ ID NOs: 5, 8, 11, 14, 17, 20, 23, 26, 44, 45, or combinations thereof.

17. The method of claim 11, wherein the recombinant gene has a nucleic acid sequence corresponding to an amino acid sequence of at least 90% similarity to SEQ ID NOs: 3, 4, 6, 7, 9, 10, 12, 13, 15, 16, 18, 19, 21, 22, 24, 25, 27, 28, 30, 31, 32, or combinations thereof.

18. A vaccine composition, comprising:
at least one oil;
at least one emulsifier;
at least one adjuvant; and
at least one antigenic peptide produced from a transgenic fungal cell, wherein the transgenic fungal cell is Thermothelomyces heterothallica.

19. The vaccine composition of claim 18, wherein the antigenic peptide sequence is selected from SEQ ID NOs: 3, 4, 6, 7, 9, 10, 12, 13, 15, 16, 18, 19, 21, 22, 24, 25, 27, 28, 30, 31, 32, or any combinations thereof.

20. A method, comprising:
providing a vaccine having a composition of claim 18; and
administering the vaccine to poultry.

21. A method for preparing a vaccine, comprising:
providing at least one antigenic peptide selected from SEQ ID NOs: 3, 4, 6, 7, 9, 10, 12, 13, 15, 16, 18, 19, 21, 22, 24, 25, 27, 28, 30, 31, 32, or any combinations thereof; and
mixing the least one antigenic peptide with at least one emulsifier and at least one adjuvant to form a mineral oil in an aqueous emulsion.
